SEQUENCE LISTING (85 sequences)

SEQ ID NO: 1 - protein, 11 aa, Human
RASQSVSSYLA

SEQ ID NO: 2 - protein, 7 aa, Human
DSSNRAT

SEQ ID NO: 3 - protein, 9 aa, Human
LQHNTFPPT

SEQ ID NO: 4 - protein, 11 aa, Human
RASQGISSRLA

SEQ ID NO: 5 - protein, 7 aa, Human
AASSLQT

SEQ ID NO: 6 - protein, 9 aa, Human
QQANRFPPT

SEQ ID NO: 7 - protein, 14 aa, Human
AGTTTDLTYYDLVS

SEQ ID NO: 8 - protein, 7 aa, Human
DGNKRPS

SEQ ID NO: 9 - protein, 10 aa, Human
NSYVSSRFYV

SEQ ID NO: 10 - protein, 13 aa, Human
SGSTSNIGTNTAN

SEQ ID NO: 11 - protein, 7 aa, Human
NNNQRPS

SEQ ID NO: 12 - protein, 12 aa, Human
AAWDDSLNGHWV

SEQ ID NO: 13 - protein, 10 aa, Human
GFTFSSYSMN

SEQ ID NO: 14 - protein, 17 aa, Human
SISSSSSYIYYADSVKG

SEQ ID NO: 15 - protein, 7 aa, Human
VTDAFDI

SEQ ID NO: 16 - protein, 10 aa, Human
GGTFSSYAIS

SEQ ID NO: 17 - protein, 18 aa, Human
GGIIPIFGTANYAQKFQG

SEQ ID NO: 18 - protein, 16 aa, Human
GYDYYDSSGVASPFDY

SEQ ID NO: 19 - DNA, 375 bp, Human
gaggtccagctggtgcagtctggggctgaggtgaagaagcctggggcctcagtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacttttaccgcggacaaatccacgagtacagcctatatggagttgaggagcctgagatctgacgacacggccgtgtattactgtgcgagaggatacgattactatgatagtagtggcgtggcttccccctttgactactggggccagggaaccctggtcaccgtctcaagc

SEQ ID NO: 20 - protein, 125 aa, Human
EVQLVQSGAEVKKPGASVKVSCKASGGTFSSYAISWVRQAPGQGLEWMGGIIPIFGTANYAQKFQGRVTFTADKSTSTAYMELRSLRSDDTAVYYCARGYDYYDSSGVASPFDYWGQGTLVTVSS

SEQ ID NO: 21 - DNA, 333 bp, Human
cagtctgtgctgactcagccaccctcagcgtctgggacccccgggcagagggtcaccatctcttgttctggaagcacctccaacatcggtactaatactgcaaactggttccagcagctcccaggaacggcccccaaactcctcatccacaataataatcagcggccctcaggggtccctgaccgattctctggctccaagtctggcacctcagcctccctggccatcagtgggctccagtctgaggatgaggctgattattactgtgcagcatgggatgacagcctgaatggccattgggtgttcggcggagggaccaagctgaccgtcctg

SEQ ID NO: 22 - protein, 111 aa, Human
QSVLTQPPSASGTPGQRVTISCSGSTSNIGTNTANWFQQLPGTAPKLLIHNNNQRPSGVPDRFSGSKSGTSASLAISGLQSEDEADYYCAAWDDSLNGHWVFGGGTKLTVL

SEQ ID NO: 23 - DNA, 348 bp, Human
gaggtgcagctggtgcagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagtcacagatgcttttgatatctggggccaagggacaatggtcaccgtctcaagc

SEQ ID NO: 24 - protein, 116 aa, Human [sequence omitted]

SEQ ID NO: 25 - DNA, 321 bp, Human
gaaattgtgatgacacagtctccagccaccctgtctttgtctccaggggaaagagccaccctctcctgcagggccagtcagagtgttagcagctacttagcctggtaccaacagaaacctggccaggctcccaggctcctcatctatgattcatccaacagggccactggcatcccagccagattcagtggcagtgggtctgggacagacttcactctcaccatcagcagcctagagcctgaagattttgcaacttattactgtctacagcataacacttttcctccgacgttcggccaagggaccaaggtggaaatcaaa

SEQ ID NO: 26 - protein, 107 aa, Human
EIVMTQSPATLSLSPGERATLSCRASQSVSSYLAWYQQKPGQAPRLLIYDSSNRATGIPARFSGSGSGTDFTLTISSLEPEDFATYYCLQHNTFPPTFGQGTKVEIK

SEQ ID NO: 27 - DNA, 348 bp, Human
gaggtccagctggtgcagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagtcacagatgcttttgatatctggggccaagggacaatggtcaccgtctcaagc

SEQ ID NO: 28 - DNA, 330 bp, Human
cagtctgccctgactcagcctgcctccctgtctgggtctcctggacagtcgatcaccatctcctgcgctggaaccaccactgatcttacatattatgaccttgtctcctggtaccaacagcacccaggccaagcacccaaactcgtgatttatgacggcaataagcggccctcaggagtttctaatcgcttctctggctccaagtctggcaacacggcctccctgacaatctctggactccaggctgaggacgaggctgattattactgcaactcatatgtaagcagcaggttttatgtcttcggaactgggaccaaggtcaccgtccta

SEQ ID NO: 29 - protein, 110 aa, Human
QSALTQPASLSGSPGQSITISCAGTTTDLTYYDLVSWYQQHPGQAPKLVIYDGNKRPSGVSNRFSGSKSGNTASLTISGLQAEDEADYYCNSYVSSRFYVFGTGTKVTVL

SEQ ID NO: 30 - DNA, 348 bp, Human
gaagtgcagctggtgcagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaaggactcactgtatctgcaaatgaacagcctgagagccgaggacacggctgtgtattactgtgcgagagtcacagatgcttttgatatctggggccaagggacaatggtcaccgtctcaagc

SEQ ID NO: 31 - protein, 116 aa, Human
EVQLVQSGGGLVKPGGSLRLSCAASGFTFSSYSMNWVRQAPGKGLEWVSSISSSSSYIYYADSVKGRFTISRDNAKDSLYLQMNSLRAEDTAVYYCARVTDAFDIWGQGTMVTVSS

SEQ ID NO: 32 - DNA, 321 bp, Human
gacatccagttgacccagtctccatcttctgtgtctgcatctgtaggagacagagtcaccatcacttgtcgggcgagtcagggtattagtagtcggttagcctggtatcagcagaaaccagggaaagcccctaagctcctgatctatgctgcatccagtttgcaaactggggtcccatcaaggttcagcggcagtggatctgggacagatttcactctcactatcagcagcctgcagcctgaagattttgcaacttactattgtcaacaggctaacaggttccctccgactttcggccctgggaccaaagtggatatcaaa

SEQ ID NO: 33 - protein, 107 aa, Human
DIQLTQSPSSVSASVGDRVTITCRASQGISSRLAWYQQKPGKAPKLLIYAASSLQTGVPSRFSGSGSGTDFTLTISSLQPEDFATYYCQQANRFPPTFGPGTKVDIK

SEQ ID NO: 34 - DNA, 333 bp, Human
cagtctgtcgtgacgcagccgccctcagtgtctggggccccagggcagagggtcaccatctcctgcactgggagccactccaacttcggggcaggaactgatgtacattggtaccaacaccttccaggaacagcccccagactcctcattcatggagacagtaatcggccctccggggtccctgaccgattctctggctccaggtctggcacctcagcctccctggccatcactgggctccgggttgaggatgaggctgattattactgtcagtcgtatgactatggcctgagaggttgggtgttcggcggcgggaccaagctgaccgtcctt

SEQ ID NO: 35 - protein, 111 aa, Human
QSVVTQPPSVSGAPGQRVTISCTGSHSNFGAGTDVHWYQHLPGTAPRLLIHGDSNRPSGVPDRFSGSRSGTSASLAITGLRVEDEADYYCQSYDYGLRGWVFGGGTKLTVL

SEQ ID NO: 36 - DNA, 321 bp, Human
gatgttgtgatgactcagtctccatcgtccctgtctgcatctgtaggggacagagtcaccatcacttgccgggcaagtcagaacattaacaactatttaaattggtatcaacagaaaccaggaaaagcccctaagctcctgatctatgctgcctccactttgcaaagtggggtcccatcaaggttcagtggcagtggatctgggacagatttcactctcaccatcaccagcctacagcctgaagattctgcaacttattactgccaacagtattcccgttatcctcccactttcggcggagggaccaaggtggagatcaca

SEQ ID NO: 37 - protein, 107 aa, Human
DVVMTQSPSSLSASVGDRVTITCRASQNINNYLNWYQQKPGKAPKLLIYAASTLQSGVPSRFSGSGSGTDFTLTITSLQPEDSATYYCQQYSRYPPTFGGGTKVEIT

SEQ ID NO: 38 - DNA, 330 bp, Human
cagtctgccctgactcagcctgcctccgtgtctgggtctcgtggacagtcgatcaccctctcctgcaccggctccagcactgatgtgggtaattataactatatctcctggtaccaacaacacccaggccaagcccccaaactcttgatttacgatgtcactagtcggccctcaggtgtttctgatcgcttctctggctccaagtcaggcctcacggcctccctgaccatctctggactccagcctgaagacgaggctgactattactgcaactcctattctgccaccgacactcttgtttttggcggagggaccaagctgaccgtccta

SEQ ID NO: 39 - protein, 110 aa, Human
QSALTQPASVSGSRGQSITLSCTGSSTDVGNYNYISWYQQHPGQAPKLLIYDVTSRPSGVSDRFSGSKSGLTASLTISGLQPEDEADYYCNSYSATDTLVFGGGTKLTVL

SEQ ID NO: 40 - DNA, 333 bp, Human
caggctgtgctgactcagccgtcctcagtgtctggggccccaggacagagggtcaccatctcctgcactgggcaaagctccaatatcggggcagattatgatgtacattggtaccagcaatttccaggaacagcccccaaactcctcatctatggtcacaacaatcggccctcaggggtccctgaccgattctctggctccaagtctggcacctcagtctccctggtcatcagtgggctccaggctgaggatgaggctgattattattgccagtcctatgacagcagtctaagtggtttggtattcggcggagggaccaaggtgaccgtccta

SEQ ID NO: 41 - protein, 111 aa, Human
QAVLTQPSSVSGAPGQRVTISCTGQSSNIGADYDVHWYQQFPGTAPKLLIYGHNNRPSGVPDRFSGSKSGTSVSLVISGLQAEDEADYYCQSYDSSLSGLVFGGGTKVTVL

SEQ ID NO: 42 - DNA, 321 bp, Human
gacatccagttgacccagtctccatcttctgtgtctgcatctgttggagacagcgtcaccatcacttgtcgggcgagtcaggatattagcagctggttagcctggtatcaacagaaaccaggggaggcccctaagctcctgatctatgctgcatcccttcttcaaagtggggtcccatcacggttcagcggcagtggatctgggacagatttcgctctcactatcaacagcctgcagcctgaagattttgcaacttacttttgtcaacaggctgacagtttccctcccaccttcggccaagggacacggctggagattaaa

SEQ ID NO: 43 - protein, 107 aa, Human
DIQLTQSPSSVSASVGDSVTITCRASQDISSWLAWYQQKPGEAPKLLIYAASLLQSGVPSRFSGSGSGTDFALTINSLQPEDFATYFCQQADSFPPTFGQGTRLEIK

SEQ ID NO: 44 - DNA, 321 bp, Human
gacatcgagttgacccagtctccatcttccgtgtctgcatctgtgggagacagagtcaccctcacttgtcgggcgagtcagagtattaagaggtggttagcctggtatcagcagaaaccagggaaggcccctaggctcctcatctatgctgcatccactttgcaaagtggggtcccatcaaggttcagcggcggtggatctgggacagatttcactctcaccatcaacagcctgcagcctgaagattttgcaatttactactgtcaacaggctaacagtttccctcccactttcggccctgggaccaaagtggatatcaaa

SEQ ID NO: 45 - protein, 107 aa, Human
DIELTQSPSSVSASVGDRVTLTCRASQSIKRWLAWYQQKPGKAPRLLIYAASTLQSGVPSRFSGGGSGTDFTLTINSLQPEDFAIYYCQQANSFPPTFGPGTKVDIK

SEQ ID NO: 46 - DNA, 333 bp, Human
cagtctgtcgtgacgcagccgccctcagtgtctggggccccagggcagagggtcaccatctcctgcagtgggagcaggtccaacatcggggcacactatgaagtccagtggtaccagcagtttccgggagcagcccccaaactcctcatctatggtgacaccaatcggccctcaggggtccctgaccgattctctgcctcccactctggcacctcagcctcccttgccatcacagggctccaggctgaggatgaggctgattattactgccagtcgtatgacaccagtctacgtggtccggtgttcggcggagggaccaagctgaccgtccta

SEQ ID NO: 47 - protein, 111 aa, Human
QSVVTQPPSVSGAPGQRVTISCSGSRSNIGAHYEVQWYQQFPGAAPKLLIYGDTNRPSGVPDRFSASHSGTSASLAITGLQAEDEADYYCQSYDTSLRGPVFGGGTKLTVL

SEQ ID NO: 48 - DNA, 333 bp, Human
cagtctgtcgtgacgcagccgccctcagtgtctggggccccagggcagagggtcaccatctcctgcactgggagcagctccaacatcgggacaggttatgatgtacattggtaccagcaggttccaggatcagcccccaaactcctcatctatgcttacaccaatcggccctcaggggtccctgaccgattctctggctccaagtctggcatgtcagcctccctggtcatcggtggtctccaggctgaggatgaggctgattattactgccagtcctttgacgacagcctgaatggtcttgtcttcggacctgggacctcggtcaccgtcctc

SEQ ID NO: 49 - protein, 111 aa, Human
QSVVTQPPSVSGAPGQRVTISCTGSSSNIGTGYDVHWYQQVPGSAPKLLIYAYTNRPSGVPDRFSGSKSGMSASLVIGGLQAEDEADYYCQSFDDSLNGLVFGPGTSVTVL

SEQ ID NO: 50 - DNA, 333 bp, Human
cagtctgtgttgacgcagccgccctcagtgtctggggccccagggcagagggtcaccatctcctgcactgggagccactccaacttcggggcaggtactgatgtccattggtaccaacaccttccaggaacagcccccagactcctcattcatggagacactcatcggccctccggggtcgctgaccgattctctggctccaggtctggcgcctcagcctccctggccatcactgggctccgggttgaggatgaggctgattattactgtcagtcgtatgactatggcctgagaggttgggtgttcggcggcgggaccaagctgaccgtcctt

SEQ ID NO: 51 - protein, 111 aa, Human
QSVLTQPPSVSGAPGQRVTISCTGSHSNFGAGTDVHWYQHLPGTAPRLLIHGDTHRPSGVADRFSGSRSGASASLAITGLRVEDEADYYCQSYDYGLRGWVFGGGTKLTVL

SEQ ID NO: 52 - DNA, 321 bp, Human
gacatccagatgacccagtctccatcttccgtgtctgcatctataggagacagagtcaccatcacttgtcgggcgagtcagggtattgacaactggttaggctggtatcagcagaaacctgggaaagcccctaaactcctgatctacgatgcatccaatttggacacaggggtcccatcaaggttcagtggaagtggatctgggacatattttactctcaccatcagtagcctgcaagctgaagattttgcagtttatttctgtcaacaggctaaagcttttcctcccactttcggcggagggaccaaggtggacatcaaa

SEQ ID NO: 53 - protein, 107 aa, Human [sequence omitted]

SEQ ID NO: 54 - protein, 13 aa, Human
TGSHSNFGAGTDV

SEQ ID NO: 55 - protein, 7 aa, Human
GDSNRPS

SEQ ID NO: 56 - protein, 11 aa, Human
QSYDYGLRGWV

SEQ ID NO: 57 - protein, 11 aa, Human
RASQNINNYLN

SEQ ID NO: 58 - protein, 7 aa, Human
AASTLQS

SEQ ID NO: 59 - protein, 9 aa, Human
QQYSRYPPT

SEQ ID NO: 60 - protein, 14 aa, Human
TGSSTDVGNYNYIS

SEQ ID NO: 61 - protein, 7 aa, Human
DVTSRPS

SEQ ID NO: 62 - protein, 10 aa, Human
NSYSATDTLV

SEQ ID NO: 63 - protein, 14 aa, Human
TGQSSNIGADYDVH

SEQ ID NO: 64 - protein, 7 aa, Human
GHNNRPS

SEQ ID NO: 65 - protein, 11 aa, Human
QSYDSSLSGLV

SEQ ID NO: 66 - protein, 10 aa, Human
RASQDISWLA

SEQ ID NO: 67 - protein, 7 aa, Human
AASLLQS

SEQ ID NO: 68 - protein, 9 aa, Human
QQADSFPPT

SEQ ID NO: 69 - protein, 11 aa, Human
RASQSIKRWLA

SEQ ID NO: 70 - protein, 7 aa, Human
AASTLQS

SEQ ID NO: 71 - protein, 9 aa, Human
QQANSFPPT

SEQ ID NO: 72 - protein, 14 aa, Human
SGSRSNIGAHYEVQ

SEQ ID NO: 73 - protein, 7 aa, Human
GDTNRPS

SEQ ID NO: 74 - protein, 11 aa, Human
QSYDTSLRGPV

SEQ ID NO: 75 - protein, 14 aa, Human
TGSSSNIGTGYDVH

SEQ ID NO: 76 - protein, 7 aa, Human
AYTNRPS

SEQ ID NO: 77 - protein, 11 aa, Human
QSFDDSLNGLV

SEQ ID NO: 78 - protein, 14 aa, Human
TGSHSNFGAGTDVH

SEQ ID NO: 79 - protein, 7 aa, Human
GDTHRPS

SEQ ID NO: 80 - protein, 11 aa, Human
QSYDYGLRGWV

SEQ ID NO: 81 - protein, 11 aa, Human
RASQGIDNWLG

SEQ ID NO: 82 - protein, 7 aa, Human
DASNLDT

SEQ ID NO: 83 - protein, 9 aa, Human
QQAKAFPPT

SEQ ID NO: 84 - DNA, 2351 bp, Human
ggtaccgagaaagaaccggctcccgagttctgggcatttcgcccggctcgaggtgcaggatgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccatgctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggaaaatcagacgacaagtattggggaaagcatcgaagtctcatgcacggcatctgggaatccccctccacagatcatgtggtataaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaa

SEQ ID NO: 85 - protein, 764 aa, Human
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSHAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLE

(19) United States
(12) Patent Application Publication
(10) Pub. No.: US 2009/0022716 A1
(43) Pub. Date: Jan. 22, 2009

(54) COMBINATION METHODS OF INHIBITING TUMOR GROWTH WITH A VASCULAR ENDOTHELIAL GROWTH FACTOR RECEPTOR ANTAGONIST

(75) Inventors: Patricia Rockwell, West Redding, CT (US); Neil I. Goldstein, Maplewood, NJ (US)

Correspondence Address: IMCLON;Lerner, David, Littenberg, Krumholz & Mentlik, LLP, 600 South Avenue West, Westfield, NJ, 07090, US

(73) Assignee: IMCLONE SYSTEMS INC., New York, NY (US)

(21) Appl. No.: 11/828,285

(22) Filed: Jul. 25, 2007

Related U.S. Application Data
(63) Continuation of application No. 10/091,300, filed on Mar. 4, 2002, now abandoned.
Application No. 10/091,300 is a continuation-in-part of application No. 09/798,689, filed on Mar. 2, 2001, now Pat. No. 6,811,779.

(51) Int. Cl. A61K 39/395; A61P 35/00

(52) U.S. Cl. 424/133.1; 424/172.1

(57) ABSTRACT

The present invention provides a method of reducing or inhibiting tumor growth in a mammal comprising treating the mammal with an effective amount of a combination of a VEGF receptor antagonist and radiation, chemotherapy, and/or an additional receptor antagonist.

This application is a continuation of application Ser. No. 10/091,300, filed Mar. 4, 2002, which is a continuation-in-part of application Ser. No. 09/798,689, filed Mar. 2, 2001, now U.S. Pat. No. 6,811,779 the entire disclosures of which are incorporated here in by reference.

### FIELD OF THE INVENTION

The present invention is directed to methods of treating tumors utilizing vascular endothelial growth factor (VEGF) receptor antagonists in combination with a chemotherapeutic agent, radiation, and/or a different growth factor receptor antagonist.

### BACKGROUND OF THE INVENTION

Angiogenesis, which refers to the formation of capillaries from pre-existing vessels in the embryo and adult organism, is known to be a key element in tumor growth, survival and metastasis. Growth factors and their receptors, including epidermal growth factor (EGF), transforming growth factor-α (TGF-α), transforming growth factor-β (TGF-β), acidic and basic fibroblast growth factor (aFGF and bFGF), platelet derived growth factor (PDGF), and vascular endothelial growth factor (VEGF), are thought to play a role in tumor angiogenesis. See Klagsbrun & D'Amore, Annual Rev. Physiol., 53: 217-239 (1991). Binding of these growth factors to their cell surface receptors induces receptor activation, which initiates and modifies signal transduction pathways and leads to cell proliferation and differentiation. VEGF, an endothelial cell-specific mitogen, is distinct among these factors in that it acts as an angiogenesis inducer by specifically promoting the proliferation of endothelial cells.

VEGF is a homodimeric glycoprotein consisting of two 23 kD subunits and is a strong inducer of vascular permeability, stimulator of endothelial cell migration and proliferation, and an important survival factor for newly formed blood vessels. Four different monomeric isoforms of VEGF exist, resulting from alternative splicing of mRNA. These include two membrane bound forms (VEGF₂₀₆ and VEGF₁₈₉) and two soluble forms (VEGF₁₆₅ and VEGF₁₂₁). In all human tissues except placenta, VEGF₁₆₅ is the most abundant isoform.

VEGF is a key regulator of vasculogenesis, which is the de novo development of new blood vessels from the differentiation of endothelial precursors (angioblasts) in situ, and is expressed in embryonic tissues (Breier et al., Development (Camb.), 114:521 (1992)), macrophages, proliferating epidermal keratinocytes during wound healing (Brown et al., J. Exp. Med., 176:1375 (1992)), and may be responsible for tissue edema associated with inflammation (Ferrara et al., Endocr. Rev., 13:18 (1992)). In situ hybridization studies have demonstrated high VEGF expression in a number of human tumor lines including glioblastoma multiforme, hemangioblastoma, central nervous system neoplasms and AIDS-associated Kaposi's sarcoma (Plate et al. (1992) Nature 359: 845-848; Plate et al. (1993) Cancer Res. 53: 5822-5827; Berkman et al. (1993) J. Clin. Invest. 91: 153-159; Nakamura et al. (1992) AIDS Weekly, 13 (1)). High levels of VEGF were also observed in hypoxia induced angiogenesis (Shweiki et al. (1992) Nature 359: 843-845).

The biological response of VEGF is mediated through its high affinity receptors, which are selectively expressed on endothelial cells during embryogenesis (Millauer, Cell, 72: 835-846 (1993)) and during tumor formation. VEGF receptors (VEGFRs) typically are class III receptor-type tyrosine kinases characterized by having several, typically 5 or 7, immunoglobulin-like loops in their amino-terminal extracellular receptor ligand-binding domains (Kaipainen et al., J. Exp. Med., 178:2077-2088 (1993)). The other two regions include a transmembrane region and a carboxy-terminal intracellular catalytic domain interrupted by an insertion of hydrophilic interkinase sequences of variable lengths, called the kinase insert domain (Tennan et al., Oncogene, 6:1677-1683 (1991). VEGFRs include fms-like tyrosine kinase receptor (flt-1), or VEGFR-1, sequenced by Shibuya et al., Oncogene, 5: 519-524 (1990), kinase insert domain-containing receptor/fetal liver kinase (KDR/flk-1), or VEGFR-2, described in WO 92/14248, filed Feb. 20, 1992, and Terman et al., Oncogene, 6: 1677-1683 (1991) and sequenced by Matthews et al., Proc. Natl. Acad. Sci. USA, 88: 9026-9030 (1991), although other receptors, such as neuropilin-1 and -2, can also bind VEGF. Another tyrosine kinase receptor, VEGFR-3 (flt-4), binds the VEGF homologues VEGF-C and VEGF-D and is more important in the development of lymphatic vessels.

High levels of Flk-1 are expressed by endothelial cells that infiltrate gliomas (Plate et al., (1992) Nature 359: 845-848). Flk-1 levels are specifically upregulated by VEGF produced by human glioblastomas (Plate et al. (1993) Cancer Res. 53: 5822-5827). The finding of high levels of Flk-1 expression in glioblastoma associated endothelial cells (GAEC) indicates that receptor activity is probably induced during tumor formation since Flk-1 transcripts are barely detectable in normal brain endothelial cells. This upregulation is confined to the vascular endothelial cells in close proximity to the tumor. Blocking VEGF activity with neutralizing anti-VEGF monoclonal antibodies (mAbs) resulted in an inhibition of the growth of human tumor xenografts in nude mice (Kim et al. (1993) Nature 362: 841-844), indicating a direct role for VEGF in tumor-related angiogenesis.

Although the VEGF ligand is upregulated in tumor cells, and its receptors are upregulated in tumor infiltrated vascular endothelial cells, the expression of the VEGF ligand and its receptors is low in normal cells that are not associated with angiogenesis. Therefore, such normal cells would not be affected by blocking the interaction between VEGF and its receptors to inhibit angiogenesis, and therefore tumor growth.

An object of the present invention is to provide VEGF receptor antagonists. A further object of this invention is to provide methods to inhibit angiogenesis and thereby to inhibit or reduce tumor growth in mammals using such VEGF receptor antagonists and, in particular, using such VEGF receptor antagonists combined with radiation, chemotherapy, or another receptor antagonist.

### BRIEF SUMMARY OF THE INVENTION

The present invention provides methods of reducing or inhibiting tumor growth in a mammal by administering an effective amount of a combination of a VEGF receptor antagonist and another receptor antagonist. Also provided by the present invention are methods of reducing or inhibiting tumor growth in a mammal by administering an effective amount of a combination of a VEGF receptor antagonist and radiation. In addition, the present invention provides methods of reducing or inhibiting tumor growth in a mammal by administering an effective amount of a combination of a VEGF receptor antagonist and a chemotherapeutic agent.

### BRIEF DESCRIPTION OF THE FIGURES

FIG. 1: Western Blot of flk-1 (VEGFR-2)/SEAPS immunoprecipitation with monoclonal antibody DC-101 demonstrating that DC-101 immunoprecipitates murine flk-1:SEAPS but not SEAPS alone.

FIGS. 2a and 2b: FIG. 2a: Competitive inhibition assay indicating the effect of anti-flk-1 (VEGFR-2) monoclonal antibody DC-101 on VEGF₁₆₅ induced phosphorylation of the flk-1 (VEGFR-2)/fms receptor in transfected 3T3 cells. FIG. 2b: Sensitivity of VEGF induced phosphorylation of the flk-1 (VEGFR-2)/fms receptor to inhibition by monoclonal antibody DC-101. C441 cells were assayed at maximal stimulatory concentrations of VEGF₁₆₅ (40 ng/ml) combined with varying levels of the antibody.

FIGS. 3a and 3b: FIG. 3a: Titration of VEGF-induced phosphorylation of the flk-1 (VEGFR-2)/fms receptor in the presence of mAb DC-101. C441 cells were stimulated with the concentrations of VEGF indicated in the presence (Lanes 1 to 4) or absence (Lanes 5 to 8) of 5 μg/ml of mAb DC-101. Unstimulated cells assayed in the presence of antibody (Lane 9) serves as the control. FIG. 3b: Densitometry scans of the level of phosphorylated receptor in each lane in FIG. 3a relative to each VEGF concentration is plotted to show the extent of mAb inhibition at excess ligand concentrations. Cell lysates were prepared for detection by anti-phosphotyrosine as described in the Examples below.

FIG. 4: Inhibition of VEGF-flk-1 (VEGFR-2)/fms activation by prebound mAb DC-101. C441 cells were stimulated with the concentrations of VEGF indicated in the absence (Lanes 3 and 4) and presence (Lanes 5 and 6) of DC-101. Unstimulated cells (Lanes 1 and 2) serve as controls. MAb was assayed using two sets of conditions. For P, cells were prebound with mAb followed by stimulation with VEGF for 15 minutes at room temperature. For C, mAb and ligand were added simultaneously and assayed as above.

FIG. 5: VEGF-induced phosphorylation of the flk-1 (VEGFR-2)/fms receptor by treatments with varying concentrations of monoclonal antibody DC-101 and conditioned media from glioblastoma cells (GB CM).

FIG. 6: FACS analysis of anti-flk-1 (VEGFR-2) mAb binding to flk-1 (VEGFR-2)/fms transfected 3T3 Cells (C441). Transfected flk-1 (VEGFR-2)/fms 3T3 cells were incubated on ice for 60 minutes with 10 μg/ml of the anti-flk-1 (VEGFR-2) mAb DC-101 or the isotype matched irrelevant anti-flk-1 mAb 23H7. Cells were washed and reincubated with 5 μg of goat anti-mouse IgG conjugated to FITC, washed, and analyzed by flow cytometry to determine antibody binding. Data shows the level of fluorescence for DC-101 to C441 cells relative to that detected with the irrelevant mAb 23H7.

FIG. 7: Saturation binding of mAb DC-101 to the flk-1 (VEGFR-2)/fms receptor on the transfected 3T3 cell line C441. Confluent C441 cells were incubated in 24 well plates with increasing concentrations of mAb DC-101 (50 ng/ml to 2 μg/ml) for two hours at 4° C. Cells were washed and incubated with 5 μg anti-rat IgG-biotin conjugate. To detect binding, cells were washed, incubated with a 1:1000 dilution of streptavidin-HRP, washed and incubated in a colormetric detection system (TMB). Data represents the absorbance at 540 nm versus increasing concentrations of mAb DC-101. The binding of the secondary antibody to cells alone was subtracted from each determination to adjust for non-specific binding. Data represents the average of three independent experiments.

FIG. 8: Immunoprecipitation of phosphorylated flk-1 (VEGFR-2)/fms from VEGF stimulated flk-1 (VEGFR-2)/fms transfected 3T3 cells. Cells were stimulated with VEGF as described in the Experimental Procedures and lysates were immunoprecipitated with irrelevant or relevant antibodies as follows: 1. rat anti-FLK2 IgG2a (mAb 2A13); 2. rat anti-flk-1 (VEGFR-2) IgG1 (mAb DC-101); 3. rat anti-FLK2 IgG1 (mAb 23H7); 4. rabbit anti-fins polyclonal antibody. Immunoprecipitated protein was subjected to SDS PAGE followed by Western blotting. The immunoprecipitation of VEGF activated receptor was detected by probing the blots with an anti-phosphotyrosine antibody.

FIG. 9: Sensitivity of VEGF-induced phosphorylation of the flk-1 (VEGFR-2)/fms receptor to inhibition by mAb DC-101. Prebound and competitive assays were performed with 40 ng/ml of VEGF at the antibody concentrations indicated. Cell lysates were prepared for receptor detection with anti-phophotyrosine as described in the Examples below.

FIG. 10: Effect of mAb DC-101 on CSF-1 induced phosphorylation of the fms receptor. In (B), the fms/FLK-2 transfected 3T3 cell line, 10A2, was stimulated with optimal stimulatory levels of CSF-1 in the absence (Lanes 3 and 4) and presence (Lanes 5 and 6) of 5 μg/ml of mAb DC-101. Unstimulated cells assayed in the absence (Lane 1) or presence (Lane 2) of antibody serve as controls. Cell lysates were prepared for detection by anti-phosphotyrosine as described in the Examples below.

FIG. 11: Specificity of mAb DC-101 neutralization of the activated flk-1 (VEGFR-2)/fms receptor. C441 cells were stimulated with 20 or 40 ng/ml of VEGF in the presence of DC-101 (IgG1) or the irrelevant anti-FLK-2 rat monoclonal antibodies 2A13 (IgG2a) or 23H7 (IgG1). Assays were performed with each antibody in the absence of VEGF (Lanes 1 to 3) and in the presence of VEGF under competitive (lanes 4 to 8) or prebound (lanes 9 to 11) conditions. Cell lysates were prepared for detection by anti-phosphotyrosine as described in the Examples below. Blots were stripped and reprobed to detect the flk-1 (VEGFR-2)/fms receptor using a rabbit polyclonal antibody to the C-terminal region of the fms receptor.

FIG. 12: Immunoprecipitation of phosphoryiated receptor bands from VEGF stimulated HUVEC cells. HUVEC cells were grown to subconfluency in endothelial growth medium (EGM) for three days without a change of medium. Receptor forms were immunoprecipated by mAb DC-101 from lysates of unstimulated cells (Lane 1), VEGF stimulated cells (lane 2), and cells stimulated with VEGF in the presence of 1 μg/ml heparin (Lane 3). Phosphorylation assays, immunoprecipitations, and detection of the phosphoryiated receptor forms were performed as described in the Experimental Procedures.

FIG. 13: Effect of mAb DC-101 on the proliferation of HUVEC cells in response to VEGF. Cells were grown for 48 hours as described in the legend to FIG. 6. Cells were then subjected to the following assay conditions: no addition to medium (untreated); a change of fresh endothelial growth medium (complete medium); the addition of 10 ng/ml of VEGF in the absence or presence of 1 μg/ml heparin; and VEGF and VEGF-heparin treated cells assayed in the presence of 1 μg/ml of DC-101. Cells were assayed for proliferation by colormetric detection at 550 nm using a cell proliferation assay kit (Promega).

FIGS. 14a and 14b FIG. 14a: Reduction in tumor growth of individual animals with DC-101 (rat anti-flk-1 monoclonal antibody). FIG. 14b: Reduction in tumor growth in individual animals with the control 2A13 group (rat anti-flk-2 monoclonal antibody).

FIG. 15: Athymic nude mice were injected subcutaneously with human glioblastoma cell line GBM-18 and divided into three groups: a PBS control, an irrelevant rat IgG1 control 23H7, and DC-101. Treatments were administered simultaneously with tumor xenografts and continued for four weeks.

FIG. 16: A graph showing the direct binding of different scFv antibodies (p1C11, p1F12, p2A6 and P2A7) to immobilized KDR (VEGFR-2).

FIG. 17: A graph showing the inhibition of binding of KDR (VEGFR-2) to immobilized VEGF₁₆₅ by different scFv antibodies (p1C11, p1F12, p2A6 and p2A7).

FIG. 18: A graph showing the inhibition of VEGF-induced HUVEC proliferation by scFv antibodies (p2A6 and p1C11).

FIG. 19: The nucleotide and deduced amino acid sequence of V_{H} and V_{L} chains of c-p1C11.

FIG. 20: A graph showing the direct binding of antibodies (c-p1C11, p1C11, p2A6) to immobilized KDR (VEGFR-2).

FIG. 21: A graph showing the FACS analysis of c-p1C11 binding to KDR-(VEGFR-2) expressing HUVEC.

FIG. 22: A graph showing the inhibition of binding of KDR (VEGFR-2) receptor to immobilized VEGF₁₆₅ by different scFv antibodies (c-p1C11, p1C11, and p2A6).

FIG. 23: A graph showing the inhibition of binding of radiolabeled VEGF₁₆₅ to immobilized KDR (VEGFR-2) receptor by c-p1C11 and cold VEGF₁₆₅.

FIG. 24: A graph showing the inhibition of VEGF-induced HUVEC proliferation by anti-KDR (VEGFR-2) antibodies (c-p1C11, p1C11).

### DETAILED DESCRIPTION OF THE INVENTION

The present invention provides methods of reducing or inhibiting tumor growth in mammals with radiation, chemotherapy, and/or an additional receptor antagonist in combination with VEGF receptor antagonists.

In a preferred embodiment, there is synergy when tumors, including human tumors, are treated with a VEGF receptor antagonist in conjunction with chemotherapeutic agents, radiation, or an additional receptor antagonist or combinations thereof. In other words, the inhibition of tumor growth by a VEGF receptor antagonist is enhanced more than expected when combined with chemotherapeutic agents, radiation, or an additional receptor antagonist or combinations thereof. Synergy may be shown, for example, by greater inhibition of tumor growth with combined treatment than would be expected from the additive effect of treatment with a VEGF receptor antagonist and a chemotherapeutic agent, radiation, or an additional receptor antagonist. Preferably, synergy is demonstrated by remission of the cancer where remission is not expected from treatment with a combination of a VEGF receptor antagonist and a chemotherapeutic agent, radiation, or an additional receptor antagonist. (See Example VIII.)

The VEGF receptor antagonist is administered before, during, or after commencing chemotherapy or radiation therapy, as well as any combination thereof, i.e. before and during, before and after, during and after, or before, during, and after commencing the chemotherapy and/or radiation therapy. For example, when the VEGF receptor antagonist is an antibody, the antibody is typically administered between 1 and 30 days, preferably between 3 and 20 days, more preferably between 5 and 12 days before commencing radiation therapy and/or chemotherapy.

### Radiation

The source of radiation, used in combination with a VEGF receptor antagonist, can be either external or internal to the patient being treated. When the source is external to the patient, the therapy is known as external beam radiation therapy (EBRT). When the source of radiation is internal to the patient, the treatment is called brachytherapy (BT).

The radiation is administered in accordance with well known standard techniques using standard equipment manufactured for this purpose, such as AECL Theratron and Varian Clinac. The dose of radiation depends on numerous factors as is well known in the art. Such factors include the organ being treated, the healthy organs in the path of the radiation that might inadvertently be adversely affected, the tolerance of the patient for radiation therapy, and the area of the body in need of treatment. The dose will typically be between 1 and 100 Gy, and more particularly between 2 and 80 Gy. Some doses that have been reported include 35 Gy to the spinal cord, 15 Gy to the kidneys, 20 Gy to the liver, and 65-80 Gy to the prostate. It should be emphasized, however, that the invention is not limited to any particular dose. The dose will be determined by the treating physician in accordance with the particular factors in a given situation, including the factors mentioned above.

The distance between the source of the external radiation and the point of entry into the patient may be any distance that represents an acceptable balance between killing target cells and minimizing side effects. Typically, the source of the external radiation is between 70 and 100 cm from the point of entry into the patient.

Brachytherapy is generally carried out by placing the source of radiation in the patient. Typically, the source of radiation is placed approximately 0-3 cm from the tissue being treated. Known techniques include interstitial, intercavitary, and surface brachytherapy. The radioactive seeds can be implanted permanently or temporarily. Some typical radioactive atoms that have been used in permanent implants include iodine-125 and radon. Some typical radioactive atoms that have been used in temporary implants include radium, cesium-137, and iridium-192. Some additional radioactive atoms that have been used in brachytherapy include americium-241 and gold-198.

The dose of radiation for brachytherapy can be the same as that mentioned above for external beam radiation therapy. In addition to the factors mentioned above for determining the dose of external beam radiation therapy, the nature of the radioactive atom used is also taken into account in determining the dose of brachytherapy.

### Chemotherapy

Chemotherapeutic agents include all chemical compounds that are effective in inhibiting tumor growth.

The administration of chemotherapeutic agents can be accomplished in a variety of ways including systemically by the parenteral and enteral routes. In one embodiment, the VEGF receptor antagonist and the chemotherapeutic agent are administered as separate molecules. In another embodiment, the VEGF receptor antagonist is attached, such as, for example, by conjugation, to a chemotherapeutic agent.

Examples of chemotherapeutic agents include alkylating agents, for example, nitrogen mustards, ethyleneimine compounds and alkyl sulphonates; antimetabolites, for example, folic acid, purine or pyrimidine antagonists; mitotic inhibitors, for example, vinca alkaloids and derivatives of podophyllotoxin; cytotoxic antibiotics; compounds that damage or interfere with DNA expression.

Additionally, chemotherapeutic agents include antibodies, biological molecules and small molecules, as described herein.

Particular examples of chemotherapeutic agents or chemotherapy include cisplatin, dacarbazine (DTIC), dactinomycin, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin, paclitaxel (taxol), docetaxel (taxotere), aldesleukin, asparaginase, busulfan, carboplatin, cladribine, dacarbazine, floxuridine, fludarabine, hydroxyurea, ifosfamide, interferon alpha, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil, taxol and combinations thereof.

### Growth Factor Receptor Antagonists

Growth factor receptor antagonists (other than VEGF receptor antagonists) that can be used in the context of the present invention include all substances that inhibit the stimulation of a growth factor receptor by a growth factor receptor ligand. Such inhibition of stimulation inhibits the growth of cells that express the growth factor receptor.

Some examples of growth factor receptors involved in tumorigenesis are the receptors for epidermal growth factor (EGFR), platelet-derived growth factor (PDGFR), insulin-like growth factor (IGFR), nerve growth factor (NGFR), and fibroblast growth factor (FGF).

Preferably, the growth factor receptor antagonist to be used in this invention is an EGFR antagonist. An EGFR antagonist, in the context of the present invention, is a biological molecule, small molecule, or any other substance that inhibits EGFR activation, thereby inhibiting the tyrosine kinase activity of EGFR, and preventing receptor autophosphorylation and the phosphorylation of other proteins involved in the various EGFR signaling pathways. By inhibition of activation of EGFR is meant any decrease in the activation of the EGFR, which need not completely prevent or stop activation of EGFR.

Moreover, inhibition of EGFR activation, as defined by the present invention, means inhibition of EGFR resulting from interaction of the EGFR antagonist and the receptor. By interaction is meant sufficient physical or chemical interaction between the EGFR antagonist and the receptor, such that tyrosine kinase activity is inhibited. One of skill in the art would appreciate that examples of such chemical interactions, which include association or bonding, are known in the art and include covalent bonding, ionic bonding, hydrogen bonding, etc., between the EGFR antagonist and the receptor. This is in contrast with an EGF antagonist, which interacts with the ligand, thereby inhibiting activation.

As is the case with other growth factors, increased EGFR activation can result from higher levels of ligand, EGFR gene amplification, increased transcription of the receptor or mutations that cause unregulated receptor signaling. Amplification of the gene encoding EGFR results in an increased number of ligands binding to the EGFR, which can further stimulate cell proliferation. EGFR may also be overexpressed in the absence of gene amplification, presumably through mutations that increase EGFR transcription, mRNA translation, or stability of the protein. EGFR mutants have been identified in gliomas, non-small-cell lung carcinomas, ovarian carcinomas and prostate carcinomas that have a constitutively active tyrosine kinase, suggesting a role for high-level EGFR activity rather than EGFR overexpression in these cancers. See, e.g., Pedersen et al., Ann. Oncol., 12(6):745-60 (2001). (Type III EGFR mutation—variously named EGFRvIII, de2-7 EGFR or AEGFR—lacks a portion of the extracellular ligand binding domain encoded by exons 2-7); see also Wikstrand et al., Cancer Res., 55:3140-3148 (1995).

In one embodiment of the present invention, the EGFR antagonist inhibits binding of EGFR to its ligand. Binding of a ligand to an external, extracellular domain of EGFR stimulates receptor dimerization, autophosphorylation of EGFR, activation of the receptor's internal, cytoplasmic tyrosine kinase domain, and initiation of multiple signal transduction pathways involved in regulation of DNA synthesis and cell division. Ligands for EGFR include, for example, EGF, TGF-α, amphiregulin, heparin-binding EGF (HB-EGF) and betarecullulin. EGF and TGF-α are thought to be the main endogenous ligands that result in EGFR-mediated stimulation, although TGF-α has been shown to be more potent in promoting angiogenesis.

In another embodiment of the present invention, the EGFR antagonist binds EGFR. It should be appreciated that the EGFR antagonist can bind externally to the extracellular portion of EGFR, which may or may not inhibit binding of the ligand, or internally to the tyrosine kinase domain. Examples of EGFR antagonists that bind EGFR include, without limitation, biological molecules, such as antibodies (and functional equivalents thereof) specific for EGFR, and synthetic kinase inhibitors that act directly on the cytoplasmic domain of EGFR, such as small molecules.

The EGFR antagonist of the present invention is preferably a biological molecule, more preferably an antibody, or functional equivalent thereof, specific for EGFR. A description of the antibodies useful in the present invention can be found in the section entitled “Antibodies.” Furthermore, following antibody binding, the EGFR-antibody complex is preferably internalized and degraded, preventing receptor re-utilization by the cell.

A known biological molecule EGFR antagonist is ERBITUX™ (IMC-C225), which is a chimeric (human/mouse) monoclonal antibody specific for EGFR. See, e.g., U.S. Pat. No. 4,943,533 (Mendelsohn et al.); U.S. Pat. No. 6,217,866 (Schlessinger et al.); U.S. application Ser. Nos. 08/973,065 (Goldstein et al.) and 09/635,974 (Teufel); WO 99/60023 (Waksal et al.) and WO 00/69459 (Waksal). The monoclonal antibody ERBITUX™ specifically binds EGFR and blocks binding of a ligand, e.g., EGF. This blockade results in inhibition of tumor growth, which includes inhibition of tumor invasion, metastases, cell repair and angiogenesis, by interfering with the effects of EGFR activation. In addition, or alternatively, the monoclonal antibody ERBITUX™ may promote internalization of the receptor-antibody complex, preventing further stimulation of the receptor by its ligand or any other mechanism.

Another example of a biological molecule EGFR antagonist is ABX-EGF, which is a fully human IgG2 monoclonal antibody specific for EGFR. ABX-EGF binds EGFR with high specificity, blocking binding of EGFR to both of its ligands, EGF and TGF-α. See, e.g., Figlin et al., Abstract 1102 presented at the 37th Annual Meeting of ASCO, San Francisco, Calif., 12-15 May 2001. The sequence and characterization of ABX-EGF, which was formerly known as clone E7.6.3, is disclosed in U.S. Pat. No. 6,235,883 (Abgenix, Inc.) at col. 28, line 62 through col. 29, line 36 and in FIG. 29-34. See Yang et al., Critical Rev. Oncol./Hematol., 38(1): 17-23, 2001.

In an alternative, but also preferred, embodiment, the EGFR antagonist of the present invention is a small molecule tyrosine kinase inhibitor. A description of small molecules can be found in the section entitled “Small Molecules.” Numerous small molecules have been described as being useful to inhibit EGFR.

For example, Spada et al., U.S. Pat. No. 5,656,655, discloses styryl substituted heteroaryl compounds that inhibit EGFR. The heteroaryl group is a monocyclic ring with one or two heteroatoms, or a bicyclic ring with 1 to about 4 heteroatoms, the compound being optionally substituted or polysubstituted. The compounds disclosed in U.S. Pat. No. 5,656,655 are incorporated herein by reference.

Spada et al., U.S. Pat. No. 5,646,153 discloses bis mono and/or bicyclic aryl heteroaryl, carbocyclic, and heterocarbocyclic compounds that inhibit EGFR. The compounds disclosed in U.S. Pat. No. 5,646,153 are incorporated herein by reference.

Bridges el al., U.S. Pat. No. 5,679,683 discloses tricyclic pyrimidine compounds that inhibit the EGFR. The compounds are fused heterocyclic pyrimidine derivatives described at column 3, line 35 to column 5, line 6. The description of these compounds at column 3, line 35 to column 5, line 6 is incorporated herein by reference.

Barker, U.S. Pat. No. 5,616,582 discloses quinazoline derivatives that have receptor tyrosine kinase inhibitory activity. The compounds disclosed in U.S. Pat. No. 5,616,582 are incorporated herein by reference.

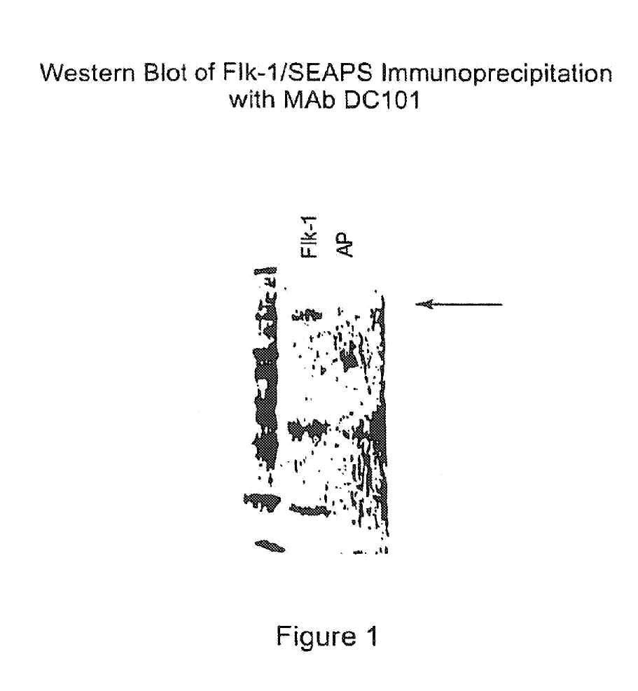

Fry et al., Science, 265: 1093-1095 (1994) discloses a compound having a structure that inhibits EGFR. The structure is shown in FIG. 1. The compound shown in FIG. 1 of the Fry et al. article is incorporated herein by reference.

Osherov et al., J. Biol. Chem., 268(15): 11, 134-42 (1993) disclose tyrphostins that inhibit EGFR/HER1 and HER2. The compounds disclosed in the Osherov et al. article, and, in particular, those in Tables I, II, III, and IV are incorporated herein by reference.

Levitzki et al., U.S. Pat. No. 5,196,446, discloses heteroarylethenediyl or heteroarylethenediylaryl compounds that inhibit EGFR. The compounds disclosed in U.S. Pat. No. 5,196,446 from column 2, line 42 to column 3, line 40 are incorporated herein by reference.

Panek et al., J. Pharma. Exp. Thera., 283: 1433-1444 (1997) disclose a compound identified as PD166285 that inhibits the EGFR, PDGFR, and FGFR families of receptors. PD166285 is identified as 6-(2,6-dichlorophenyl)-2-(4-(2-diethylaminoethoxy)phenylamino)-8-methyl-8H-pyrido(2,3-d)pyrimidin-7-one having the structure shown in FIG. 1 on page 1436. The compound described in FIG. 1 on page 1436 of the Panek et al. article is incorporated herein by reference

One example of a small molecule EGFR antagonist is IRESSA™ (ZD1939), which is a quinozaline derivative that functions as an ATP-mimetic to inhibit EGFR. See U.S. Pat. No. 5,616,582 (Zeneca Limited); WO 96/33980 (Zeneca Limited) at p. 4; see also, Rowinsky et al., Abstract 5 presented at the 37th Annual Meeting of ASCO, San Francisco, Calif., 12-15 May 2001; Anido et al., Abstract 1712 presented at the 37th Annual Meeting of ASCO, San Francisco, Calif., 12-15 May 2001.

TARCEVA™ is another example of a small molecule EGFR antagonist. TARCEVA™ (OSI-774) is a 4-substituted phenylamino quinozaline derivative [6,7-Bis(2-methoxy-ethoxy)-quinazolin-4-yl]-(3-ethynyl-phenyl)amine hydrochloride] EGFR inhibitor, which is described in WO 96/30347 (Pfizer Inc.) at, for example, page 2, line 12 through page 4, line 34 and page 19, lines 14-17. See also Moyer et al., Cancer Res., 57: 4838-48 (1997); Pollack et al., J. Pharmacol., 291: 739-48 (1999). TARCEVA™ may function by inhibiting phosphorylation of EGFR and its downstream PI3/Akt and MAP (mitogen activated protein) kinase signal transduction pathways resulting in p27-mediated cell-cycle arrest. See Hidalgo et al., Abstract 281 presented at the 37th Annual Meeting of ASCO, San Francisco, Calif., 12-15 May 2001.

Many other small molecules are known to inhibit EGFR. Some examples of small molecule EGFR antagonists are described in WO 91/116051, WO 96/30347, WO 96/33980, WO 97/27199 (Zeneca Limited), WO 97/30034 (Zeneca Limited), WO 97/42187 (Zeneca Limited), WO 97/49688 (Pfizer Inc.), WO 98/33798 (Warner Lambert Company), WO 00/18761 (American Cyanamid Company), and WO 00/31048 (Warner Lambert Company). Examples of specific small molecule EGFR antagonists include Cl-1033, which is a quinozaline (N-[4-(3-chloro-4-fluoro-phenylamino)-7-(3-morpholin-4-yl-propoxy)-quinazolin-6-yl]-acrylamide) inhibitor of tyrosine kinases, particularly EGFR and is described in WO 00/31048 (Warner-Lambert Company) at page 8, lines 22-6; PKI166, which is a pyrrolopyrimidine inhibitor of EGFR and is described in WO 97/27199 (Novartis AG) at pages 10-12; GW2016, which is an inhibitor of EGFR and HER2; and EκB569.

Additional EGFR antagonists can easily be determined using well-known methods. The EGFR antagonists of the present invention inhibit the tyrosine kinase activity of EGFR, which generally involves phosphorylation events. Accordingly, phosphorylation assays are useful in determining antagonists useful in the context of the present invention. Some assays for tyrosine kinase activity are described in Panek et al. (1997) and Batley et al., Life Sci., 62: 143-50 (1998). In addition, methods specific for detection of EGFR expression can be utilized. These include immunohistochemistry (IHC) for detection of protein expression, fluorescence in situ hybridization (FISH) for detection of gene amplification, competitive radioligand binding assays, solid matrix blotting techniques, such as Northern and Southern blots, reverse transcriptase polymerase chain reaction (RT-PCR) and ELISA. See, e.g., Grandis et al., Cancer, 78:1284-1292. (1996); Shimizu et al., Japan J. Cancer Res., 85:567-571 (1994); Sauter et al., Am. J. Path., 148:1047-1053 (1996); Collins, Glia, 15:289-296 (1995); Radinsky et al., Clin. Cancer Res., 1:19-31 (1995); Petrides et al., Cancer Res., 50:3934-3939 (1990); Hoffmann et al., Anticancer Res., 17:4419-4426 (1997); Wikstrand et al., Cancer Res., 55:3140-3148 (1995).

### VEGF Receptor Antagonists

In one embodiment, the VEGF receptor antagonist binds specifically to an epitope on the extracellular domain of a VEGF receptor. The extracellular domain of a VEGF receptor is the ligand-binding domain. The ligand-binding domain may be found at either end of the receptor, but is normally found at the amino-terminal end.

Some examples of VEGF receptors include the protein tyrosine kinase receptors referred to in the literature as flt-1 (VEGFR-1), KDR and flk-1 (VEGFR-2). Unless otherwise stated or clearly suggested otherwise by context, this specification will follow the customary literature nomenclature of VEGF receptors. KDR (VEGFR-2) will be referred to as the human form of a VEGF receptor having MW 180 kD (Terman et al., above). Flk-1 (VEGFR-2) will be referred to as the murine homolog of KDR (Matthews et al., above). Flt-1 (VEGFR-1) will be referred to as a form of VEGF receptor different from, but related to, the KDR/flk-1 (VEGFR-2) receptor. See Shibuya et al., above.

Other VEGF receptors include those that can be cross-link labeled with VEGF, or that can be co-immunoprecipitated with KDR (VEGFR-2). Some known forms of these VEGF receptors have molecular weights of approximately 170 KD, 150 KD, 130-135 KD, 120-125 KD and 85 KD. See, for example, Quinn et al. Proc. Nat'l Acad. Sci. USA, 90: 7533-7537 (1993); Scher et al., J. Biol. Chem., 271: 5761-5767 (1996).

The VEGF receptor is usually bound to a cell, such as an endothelial cell. The VEGF receptor may also be bound to a non-endothelial cell, such as a tumor cell. Alternatively, the VEGF receptor may be free from the cell, preferably in soluble form.

The antagonists of the present invention neutralize VEGF receptors. In this specification, neutralizing a receptor means inactivating the intrinsic kinase activity of the receptor to transduce a signal. A reliable assay for VEGF receptor neutralization is the inhibition of receptor phosphorylation.

The present invention is not limited by any particular mechanism of VEGF receptor neutralization. At the time of filing this application, the mechanism of VEGF receptor neutralization by antibodies was not well understood, and the mechanism followed by one antagonist is not necessarily the same as that followed by another antagonist. Some possible mechanisms include preventing binding of the VEGF ligand to the extracellular binding domain of the VEGF receptor, and preventing dimerization or oligomerization of receptors. Other mechanisms cannot, however, be ruled out.

A VEGF receptor (or VEGFR) antagonist, in the context of the present invention, is a biological molecule, small molecule, or any other substance that inhibits the VEGFR subfamily of receptors. By inhibition of activation of the VEGFR subfamily of receptors is meant any decrease in the activation of the VEGFR. That is, the prevention of activation need not completely stop activation of the VEGFR. Moreover, inhibition of VEGFR activation, as defined by the present invention, means inhibition of the VEGFR antagonist following interaction of the VEGFR antagonist and VEGFR. By association is meant sufficient physical or chemical interaction between the VEGFR antagonist and VEGFR that the receptor's tyrosine kinase activity is inhibited. One of skill in the art would appreciate that examples of such chemical interactions, which include association or bonding, are known in the art and include covalent bonding, ionic bonding, hydrogen bonding, etc. Accordingly, the VEGFR antagonists of the present invention inhibit the tyrosine kinase activity of the receptor, which prevents autophosphorylation of the receptor and phosphorylation of various other proteins involved in the VEGFR signaling pathways. Such pathways, which are involved in regulation of vasculogenesis and angiogenesis, include any of the following: the phospholipase Cγ (PLCγ) pathway or the phosphatidylinositol 3′ kinase (PI3-K)/Akt and mitogen activated protein kinase (MAPK) pathway. See, e.g., Larrivée et al., Int'l J. Mol. Med., 5: 447-56 (2000).

The VEGFR subfamily of receptors is characterized by the presence of seven immunoglobulin-like loops in the extracellular domain, a single transmembrane region and a split tyrosine kinase domain in the intracellular region (class III receptor tyrosine kinases). There are several known members of the VEGFR subfamily of receptors, examples of which include VEGFR-1, VEGFR-2, and VEGFR-3. It is generally believed that KDR (VEGFR-2) is the main VEGF signal transducer that results in endothelial cell proliferation, migration, differentiation, tube formation, increase of vascular permeability, and maintenance of vascular integrity, VEGFR-1 possesses a much weaker kinase activity, and is unable to generate a mitogenic response when stimulated by VEGF—although it binds to VEGF with an affinity that is approximately 10-fold higher than KDR (VEGFR-2). VEGFR-1 is also been implicated in VEGF and placenta growth factor (P1GF) induced migration of monocytes and macrophages and production of tissue factor.

As is the case with EGFR described above, increased VEGFR activation can result from higher levels of ligand, VEGFR gene amplification, increased transcription of the receptor or mutations that cause unregulated receptor signaling.

In one embodiment of the present invention, the VEGFR antagonist inhibits binding of VEGFR to its ligand. Binding of a ligand to an external, extracellular domain of VEGFR stimulates receptor dimerization, autophosphorylation of VEGFR, activation of the receptor's internal, cytoplasmic tyrosine kinase domain, and initiation of multiple signal transduction pathways involved in regulation of vasculogenesis and angiogenesis.

Ligands for VEGFR include VEGF and its homologues P1GF, VEGF-B, VEGF-C, VEGF-D, and VEGF-E. For example, P1GF, which is a dimeric secreted factor than only binds VEGFR-1, is produced in large amounts by villous cytotrophoblast, sincytiotrophoblast and extravillous trophoblast and has close amino acid homology to VEGF. Three isoforms exist in humans, P1GF-1, P1GF-2, and P1GF-3. Studies with P1GF-deficient mice demonstrate that this growth factor is not involved in angiogenesis per se, but rather, specifically modulates the angiogenic and permeability effects of VEGF during pathological situations. Also, VEGF-D is closely related to VEGF-C by virtue of the presence of N- and C-terminal extensions that are not found in other VEGF family members. In adult human tissues, VEGF-D mRNA is most abundant in heart, lung, skeletal muscle, colon, and small intestine. Analyses of VEGF-D receptor specificity revealed that VEGF-D is a ligand for both VEGFR-2 (Flk1) and VEGFR-3 (Flt4) and can activate these receptors; however, VEGF-D does not bind to VEGFR-1. In addition, VEGF-D is a mitogen for endothelial cells.

In another embodiment of the present invention, the VEGFR antagonist binds VEGFR. It should be appreciated that the VEGFR antagonist can bind externally to the extracellular portion of VEGFR, which may or may not inhibit binding of the ligand, or internally to the tyrosine kinase domain. Examples of VEGFR antagonists that bind VEGFR include, without limitation, biological molecules, such as receptor ribozymes and antibodies (or functional equivalents thereof) specific for VEGFR, and synthetic kinase inhibitors that act directly on the cytoplasmic domain of VEGFR, such as small molecules. Preferably, the VEGFR antagonist of the present invention is a biological molecule and more preferably, an antibody, or functional equivalent thereof, specific for VEGFR, details of which are described in more detail below. Alternatively, the VEGFR antagonist of the present invention is a small molecule kinase inhibitor, details are described below.

In one preferred embodiment, the VEGF receptor antagonist binds specifically to VEGFR-1. Particularly preferred are antigen-binding proteins that bind to the extracellular domain of VEGFR-1 and block binding by one or both of its ligands, VEGF and P1GF, and/or neutralize VEGF-induced or P1GF-induced activation of VEGFR-1. For example, mAb 6.12 is a scFv that binds to soluble and cell surface-expressed VEGFR-1. ScFv 6.12 comprises the V_{L} and V_{H} domains of mouse monoclonal antibody mAb 6.12. A hybridoma cell line producing mAb 6.12 has been deposited as ATCC number PTA-3344. The deposit was made under the provisions of the Budapest Treaty on the International Recognition of the Deposit of Microorganisms for the Purposes of Patent Procedure and the regulations thereunder (Budapest Treaty). This assures maintenance of a viable culture for 30 years from date of deposit. The organisms will be made available by ATCC under the terms of the Budapest Treaty, and subject to an agreement between Applicants and ATCC, which assures unrestricted availability upon issuance of the pertinent U.S. patent. Availability of the deposited strains is not to be construed as a license to practice the invention in contravention of the rights granted under the authority of any government in accordance with its patent laws.

Other preferred antibodies are described in the Examples, specifically Examples XII, XIII, and XIV, and in SEQ ID NO:1-83. Moreover, some of these preferred VEGFR antibody antagonists are also described in Lu et al., Int'l J. Cancer, 97: 393-399 (2002).

There also exist various hybridomas that produce VEGFR-2 antibodies. For example, a hybridoma cell line producing rat anti-mouse VEGFR-2 monoclonal antibody (DC101) was deposited as ATCC HB 11534; a hybridoma cell line (M25.18A1) producing mouse anti-mouse VEGFR-2 monoclonal antibody mAb 25 was deposited as ATCC HB 12152; a hybridoma cell line (M73.24) producing mouse anti-mouse VEGFR-2 monoclonal antibody mAb 73 was deposited as ATCC HB 12153.

In addition, there are various hybridomas that produce anti-VEGFR-1 antibodies include, but not limited to, hybridomas KM1730 (deposited as FERM BP-5697), KM1731 (deposited as FERM BP-5718), KM1732 (deposited as FERM BP-5698), KM1748 (deposited as FERM BP-5699), KM1750 (deposited as FERM BP-5700) disclosed in WO 98/22616, WO 99/59636, Australian accepted Application No. AU 1998 50666 B2, and Canadian Application No. CA 2328893.

Many other VEGFR antagonists are known in the art. Some examples of VEGFR antagonists are described in U.S. Pat. Nos. 5,185,438; 5,621,090; 5,283,354; 5,270,458; 5,367,057; 5,548,065; 5,747,651; 5,912,133; U.S. application Ser. Nos. 09/208,786, abandoned; and 09/919,408, pending (all to Lemischka et al.); U.S. Pat. No. 5,840,301 (Rockwell et al.); U.S. Pat. Nos. 5,861,449; 5,874,542; 6,448,007; 5,955,311; 6,365,157 and U.S. application Ser. No. 09/789,689, allowed (all to Rockwell et al.); U.S. application Ser. No. 09/540,770, pending (Witte et al.); and PCT/US01/06966 (Liao et al.); U.S. Pat. No. 5,861,301 (Terman et al.), Terman et al. Oncogene 6: 1677-1683 (September 1991), WO 94/10202 (Ferrar et al.), and WO 95/21865 (Ludwig) disclose VEGFR antagonists and, specifically, anti-VEGFR-2 antibodies. In addition, PCT/US95/01678 (Kyowa Hakko), describes anti-VEGFR-2 antibodies. Anti-VEGFR antibodies are also described in U.S. application Ser. No. 09/976,787, pending (Zhu et al.). U.S. Pat. Nos. 6,177,401 (Ullrich et al.), 5,712,395 (App et al.), and 5,981,569 (App et al.) describe VEGFR antagonists that are organic molecules. In addition, bi-specific antibodies (BsAbs), which are antibodies that have two different antigen-binding specificities or sites, directed to KDR (VEGFR-2) and VEGFR-1 are known. See, e.g., U.S. application Ser. No. 09/865,198, abandoned (Zhu); 60/301,299 (Zhu), now U.S. application Ser. No. 10/482,630, pending.

Hennequin et al. in J. Med. Chem. 42, 5369-5389 (1999) disclose certain quinazolines, quinolines and cinnolines as being useful as VEGF receptor antagonists. See also Annie et al., Journal of Acquired Immune Deficiency Syndromes and Human Retrovirology 17, A41 (1998). The VEGF receptor antagonists disclosed in the Hennequin et al. article are incorporated herein by reference.

Additionally, App et al. (U.S. Pat. No. 5,849,742) discloses small molecule derivatives of quinazoline, quinoxiline, substituted aniline, isoxazoles, acrylonitrile and phenylacrylonitrile compounds which act as tyrosine kinase inhibitors. The small molecules described by Hennequin et al., Annie et al., and App et al. are included in the present invention as VEGF receptor antagonists.

Furthermore, assays for the determination of VEGFR antagonists are well known in the art and, therefore, alternate antagonists suitable for use in the present invention can be readily identified. The VEGFR antagonists of the present invention inhibit the tyrosine kinase activity of VEGFR, which generally involves phosphorylation events. Accordingly, phosphorylation assays are useful in determining VEGFR antagonists in the context of the present invention. Some assays for tyrosine kinase activity are described in Panek et al., J. Pharmacol. Exp. Thera., 283: 1433-44 (1997) and Batley et al., Life Sci., 62: 143-50 (1998). In addition, methods specific for detection of VEGFR expression can be utilized.

### Antibodies

The antibodies of the present invention may be produced by methods known in the art. These methods include the immunological method described by Kohler and Milstein, Nature, 256: 495-497 (1975) and Campbell, Monoclonal Antibody Technology, The Production and Characterization of Rodent and Human Hybridomas, Burdon et al., Eds., Laboratory Techniques in Biochemistry and Molecular Biology, Volume 13, Elsevier Science Publishers, Amsterdam (1985); as well as by the recombinant DNA method described by Huse et al., Science, 246, 1275-1281 (1989).

The antibodies of the present invention can be monoclonal or polyclonal antibodies or any other suitable type of an antibody, such as a fragment or a derivative of an antibody, a single chain antibody (scFv) or a synthetic homolog of the antibody, provided that the antibody has the same binding characteristics as, or that have binding characteristics comparable to, those of the whole antibody. As used herein, unless otherwise indicated or clear from the context, antibody domains, regions and fragments are accorded standard definitions as are well known in the art. See, e.g., Abbas et al., Cellular and Molecular Immunology, W.B. Saunders Company, Philadelphia, Pa. (1991). Preferably, the antibodies of the subject invention are monoclonal antibodies.

Antibody fragments can be produced by cleaving a whole antibody, or by expressing DNA that encodes the fragment. Fragments of antibodies may be prepared by methods described by Lamoyi et al., J. Immunol. Methods, 56: 235-243 (1983) and by Parham, J. Immunol. 131: 2895-2902 (1983). Such fragments may contain one or both Fab fragments or the F(ab′)₂ fragment. Such fragments may also contain single-chain fragment variable region antibodies, i.e. scFv, dibodies, or other antibody fragments. Methods of producing such functional equivalents are disclosed in PCT Application WO 93/21319, European Patent Application No. 239,400; PCT Application WO 89/09622; European Patent Application 338,745; and European Patent Application EP 332,424.

Single chain antibodies (scFv) are polypeptides that consist of the variable region of the heavy chain of the antibody linked to the variable region of the light chain with or without an interconnecting linker. Thus, the scFv comprises the entire antibody-combining site. These chains may be produced in bacteria, or in eukaryotic cells. An example of a single chain antibody is p1C11. (See Example IX below.) P1C11 was shown to block VEGF-KDR (VEGF-VEGFR-2) interaction and inhibit VEGF-stimulated receptor phosphorylation and mitogenesis of HUVEC. This scFv binds both soluble KDR (VEGFR-2) and cell surface-expressed KDR (VEGFR-2) on HUVEC. The sequence p1C11 of is shown as SEQ ID No: 21. The single chain antibodies described above can be built up into a chimerized or humanized antibody by methods known in the art; e.g., see example IX-3 below. One example of a chimerized scFv is chimerized p1C11, i.e., c-p1C11.

Preferably the antibody fragments contain all six complementarity-determining regions of the whole antibody, although fragments containing fewer than all of such regions, such as three, four or five CDRs, may also be functional. If the antibody fragment is too short to be immunogenic, it may be conjugated to a carrier molecule. Some suitable carrier molecules include keyhole limpet hemocyanin and bovine serum albumen. Conjugation may be carried out by methods known in the art.

Antibodies of the present invention also include those for which binding characteristics have been improved by direct mutation, methods of affinity maturation, phage display, or chain shuffling. Affinity and specificity may be modified or improved by mutating CDRs and screening for antigen binding sites having the desired characteristics (see, e.g., Yang et al., J. Mol. Bio., 254: 392-403 (1995)). CDRs are mutated in a variety of ways. One way is to randomize individual residues or combinations of residues so that in a population of otherwise identical antigen binding sites, all twenty amino acids are found at particular positions. Alternatively, mutations are induced over a range of CDR residues by error prone PCR methods (see, e.g., Hawkins et al., J. Mol. Bio., 226: 889-896 (1992)). Phage display vectors containing heavy and light chain variable region genes are propagated in mutator strains of E. coli (see, e.g., Low et al., J. Mol. Bio., 250: 359-368 (1996)). These methods of mutagenesis are illustrative of the many methods known to one of skill in the art.

The antibodies of the present invention can also be chimeric antibodies having a variable region of an antibody of one species, for example, a mouse, and a constant region of an antibody of a different species, for example, a human. Alternatively, the antibodies of the present invention can be humanized antibodies having hypervariable or complementarity-determining regions (CDRs) of an antibody from one species, for example, a mouse, and framework variable regions and a constant region of a human antibody. Also alternatively, the antibodies of the present invention can be human antibodies having both a constant region and a variable region of a human antibody.

Antibodies, and particularly monoclonal antibodies, can be produced by methods known in the art. Examples for production of antibodies include, but are not limited to, production in hybridoma cells and transformation of mammalian cells with DNA encoding the receptor antagonist. These methods are described in various publications, including the immunological method described by Kohler and Milstein, Nature, 256: 495-497 (1975) and Campbell in “Monoclonal Antibody Technology, The Production and Characterization of Rodent and Human Hybridomas” in Burdon et al., Eds., Laboratory Techniques in Biochemistry and Molecular Biology, Volume 13, Elsevier Science Publishers, Amsterdam (1985); as well as by the recombinant DNA methods described by Huse et al. in Science, 246: 1275-1281 (1989).

Equivalents of antibodies are also prepared by methods known in the art. For example, fragments of antibodies may be prepared enzymatically from whole antibodies. Preferably, equivalents of antibodies are prepared from DNA encoding such equivalents. DNA encoding fragments of antibodies may be prepared by deleting all but the desired portion of the DNA that encodes the full-length antibody. DNA encoding chimerized antibodies may be prepared by recombining DNA encoding human constant regions, derived substantially or exclusively from the corresponding human antibody regions, and DNA encoding variable regions, derived substantially or exclusively from the sequence of the variable region of a mammal other than a human. DNA encoding humanized antibodies may be prepared by recombining DNA encoding constant regions and variable regions other than the complementarity determining regions (CDRs), derived substantially or exclusively from the corresponding human antibody regions, and DNA encoding CDRs, derived substantially or exclusively from a mammal other than a human.

Suitable sources of DNA molecules that encode fragments of antibodies include cells, such as hybridomas, that express the full-length antibody. The fragments may be used by themselves as antibody equivalents, or may be recombined into equivalents, as described above. The DNA deletions and recombinations described in this section may be carried out by known methods, such as those described in the published patent applications listed above in the section entitled “Functional Equivalents of Antibodies” and/or other standard recombinant DNA techniques, such as those described below.

Preferred host cells for transformation of vectors and expression of the receptor antagonists of the present invention are mammalian cells, e.g., COS-7 cells, Chinese hamster ovary (CHO) cells, and cell lines of lymphoid origin such as lymphoma, myeloma, or hybridoma cells. Other eukaryotic host, such as yeasts, can be alternatively used. For example, mouse fetal liver stromal cell line 2018 binds APtag-flk 1 and APtag-flk-2 fusion proteins, i.e., contains ligands of VEGFR-2 and flk-2 (ATCC, Manassas, Va., CRL 10907), human fetal spleen cell line Fsp 62891 contains flk-2 ligand (ATCC CRL 10935), and human stromal fetal thymus cell line, F.thy 62891, contains VEGFR-2 ligand (ATCC CRL 10936).

The transformed host cells are cultured by methods known in the art in a liquid medium containing assimilable sources of carbon (carbohydrates such as glucose or lactose), nitrogen (amino acids, peptides, proteins or their degradation products such as peptones, ammonium salts or the like), and inorganic salts (sulfates, phosphates and/or carbonates of sodium, potassium, magnesium and calcium). The medium furthermore contains, for example, growth-promoting substances, such as trace elements, for example iron, zinc, manganese and the like.

Where it is desired to express a gene construct in yeast, a suitable selection gene for use in yeast is the trp1 gene present in the yeast plasmid YRp7. Stinchcomb et al. Nature, 282: 39 (1979); Kingsman et al., Gene, 7: 141 (1979). The trp1 gene provides a selection marker for a mutant strain of yeast lacking the ability to grow in tryptophan, for example, ATCC No. 44076 or PEP4-1. Jones, Genetics, 85: 12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

Also alternatively, the DNA encoding the receptor antagonist can be cloned into vectors derived from viruses such as adenovirus, adeno-associated virus, herpesvirus, retrovirus or lentivirus. Gene expression is controlled by inducible or uninducible regulatory sequences.

Briefly, a suitable source of cells containing nucleic acid molecules that express the desired DNA, such as an antibody, antibody equivalent or VEGF receptor, is selected. Total RNA is prepared by standard procedures from a suitable source. The total RNA is used to direct cDNA synthesis. Standard methods for isolating RNA and synthesizing cDNA are provided in standard manuals of molecular biology such as, for example, those described above.

The cDNA may be amplified by known methods. For example, the cDNA may be used as a template for amplification by polymerase chain reaction (PCR); see Saiki et al., Science, 239, 487 (1988) or Mullis et al., U.S. Pat. No. 4,683,195. The sequences of the oligonucleotide primers for the PCR amplification are derived from the known sequence be amplified. The oligonucleotides are synthesized by methods known in the art. Suitable methods include those described by Caruthers in Science 230, 281-285 (1985).

A mixture of upstream and downstream oligonucleotides are used in the PCR amplification. The conditions are optimized for each particular primer pair according to standard procedures. The PCR product is analyzed, for example, by electrophoresis for cDNA having the correct size, corresponding to the sequence between the primers. Alternatively, the coding region may be amplified in two or more overlapping fragments. The overlapping fragments are designed to include a restriction site permitting the assembly of the intact cDNA from the fragments.

In order to isolate the entire protein-coding regions for the VEGF receptors, for example, the upstream PCR oligonucleotide primer is complementary to the sequence at the 5′ end, preferably encompassing the ATG start codon and at least 5-10 nucleotides upstream of the start codon. The downstream PCR oligonucleotide primer is complementary to the sequence at the 3′ end of the desired DNA sequence. The desired DNA sequence preferably encodes the entire extracellular portion of the VEGF receptor, and optionally encodes all or part of the transmembrane region, and/or all or part of the intracellular region, including the stop codon.

The DNA to be amplified, such as that encoding antibodies, antibody equivalents, or VEGF receptors, may also be replicated in a wide variety of cloning vectors in a wide variety of host cells. The host cell may be prokaryotic or eukaryotic.

The vector into which the DNA is spliced may comprise segments of chromosomal, non-chromosomal and synthetic DNA sequences. Some suitable prokaryotic cloning vectors include plasmids from E. coli, such as colE1, pCR1, pBR322, pMB9, pUC, pKSM, and RP4. Prokaryotic vectors also include derivatives of phage DNA such as M13 and other filamentous single-stranded DNA phages. A preferred vector for cloning nucleic acid encoding the VEGF receptor is the Baculovirus vector.

The vector containing the DNA to be expressed is transfected into a suitable host cell. The host cell is maintained in an appropriate culture medium, and subjected to conditions under which the cells and the vector replicate. The vector may be recovered from the cell. The DNA to be expressed may be recovered from the vector.

The DNA to be expressed, such as that encoding antibodies, antibody equivalents, or receptors, may be inserted into a suitable expression vector and expressed in a suitable prokaryotic or eucaryotic host cell.

For example, the DNA inserted into a host cell may encode the entire extracellular portion of the VEGF receptor, or a soluble fragment of the extracellular portion of the VEGF receptor. The extracellular portion of the VEGF receptor encoded by the DNA is optionally attached at either, or both, the 5′ end or the 3′ end to additional amino acid sequences. The additional amino acid sequences may be attached to the VEGF receptor extracellular region in nature, such as the leader sequence, the transmembrane region and/or the intracellular region of the VEGF receptor. The additional amino acid sequences may also be sequences not attached to the VEGF receptor in nature. Preferably, such additional amino acid sequences serve a particular purpose, such as to improve expression levels, secretion, solubility, or immunogenicity.

Vectors for expressing proteins in bacteria, especially E. coli, are known. Such vectors include the PATH vectors described by Dieckmann and Tzagoloff in J. Biol. Chem. 260, 1513-1520 (1985). These vectors contain DNA sequences that encode anthranilate synthetase (TrpE) followed by a polylinker at the carboxy terminus. Other expression vector systems are based on beta-galactosidase (pEX); lambda P_{L}; maltose binding protein (pMAL); and glutathione S-transferase (pGST)—see Gene 67, 31 (1988) and Peptide Research 3, 167 (1990).

Vectors useful in yeast are available. A suitable example is the 2μ plasmid.

Suitable vectors for expression in mammalian cells are also known. Such vectors include well-known derivatives of SV-40, adenovirus, retrovirus-derived DNA sequences and shuttle vectors derived from combination of functional mammalian vectors, such as those described above, and functional plasmids and phage DNA.

Further eukaryotic expression vectors are known in the art (e.g., P. J. Southern and P. Berg, J. Mol. Appl. Genet., 1, 327-341 (1982); Subramani et al., Mol. Cell. Biol., 1: 854-864 (1981); Kaufmann and Sharp, “Amplification And Expression of Sequences Cotransfected with a Modular Dihydrofolate Reductase Complementary DNA Gene,” J. Mol. Biol. 159, 601-621 (1982); Kaufmann and Sharp, Mol. Cell. Biol. 159, 601-664 (1982); Scahill et al., “Expression And Characterization Of The Product Of A Human Immune Interferon DNA Gene In Chinese Hamster Ovary Cells,” Proc. Nat'l Acad. Sci. USA 80, 4654-4659 (1983); Urlaub and Chasin, Proc. Nat'l Acad. Sci. USA 77, 4216-4220, (1980).

The expression vectors useful in the present invention contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Vectors containing the control signals and DNA to be expressed, such as that encoding antibodies, antibody equivalents, or VEGF receptors, are inserted into a host cell for expression. Some useful expression host cells include well-known prokaryotic and eukaryotic cells. Some suitable prokaryotic hosts include, for example, E. coli, such as E. coli SG-936, E. coli HB 101, E. coli W3110, E. coli X1776, E. coli X2282, E. coli DH1, and E. coli MRC1, Pseudomonas, Bacillus, such as Bacillus subtilis, and Streptomyces. Suitable eukaryotic cells include yeast and other fungi, insect, animal cells, such as COS cells and CHO cells, human cells and plant cells in tissue culture.

Following expression in a host cell maintained in a suitable medium, the polypeptide or peptide to be expressed, such as that encoding antibodies, antibody equivalents, or VEGF receptors, may be isolated from the medium, and purified by methods known in the art. If the polypeptide or peptide is not secreted into the culture medium, the host cells are lysed prior to isolation and purification.

In addition, the antibodies of the invention may be prepared by immunizing a mammal with a soluble receptor. The soluble receptors may be used by themselves as immunogens, or may be attached to a carrier protein or to other objects, such as beads, i.e. sepharose beads. After the mammal has produced antibodies, a mixture of antibody-producing cells, such as the splenocytes, is isolated. Monoclonal antibodies may be produced by isolating individual antibody-producing cells from the mixture and making the cells immortal by, for example, fusing them with tumor cells, such as myeloma cells. The resulting hybridomas are preserved in culture, and express monoclonal antibodies, which are harvested from the culture medium.

The antibodies may also be prepared from receptors bound to the surface of cells that express the specific receptor of interest. The cell to which the receptors are bound may be a cell that naturally expresses the receptor, such as a vascular endothelial cell for VEGFR. Alternatively, the cell to which the receptor is bound may be a cell into which the DNA encoding the receptor has been transfected, such as 3T3 cells, which have been transfected with VEGFR.

A receptor may be used as an immunogen to raise an antibody of the invention. The receptor peptide may be obtained from natural sources, such as from cells that express the receptors. For example, the VEGF receptor peptide may be obtained from vascular endothelial cells. Alternatively, synthetic receptor peptides may be prepared using commercially available machines, in such an embodiment, the VEGF receptor amino acid sequence can be provided by, for example, Shibuya et al., Oncogene 5, 519-524 (1990) for flt-1 (VEGFR-1); PCT/US92/01300 and Terman et al., Oncogene 6:1677-1683 (1991) for KDR (VEGFR-2); and Matthews et al. Proc. Nat'l Acad. Sci. USA, 88:9026-9030 (1991) for flk-1.

As a further alternative, DNA encoding a receptor, such as a cDNA or a fragment thereof, may be cloned and expressed and the resulting polypeptide recovered and used as an immunogen to raise an antibody of the invention. For example, in order to prepare the VEGF receptors against which the antibodies are made, nucleic acid molecules that encode the VEGF receptors of the invention, or portions thereof, especially the extracellular portions thereof, may be inserted into known vectors for expression in host cells using standard recombinant DNA techniques, such as those described below. Suitable sources of such nucleic acid molecules include cells that express VEGF receptors, i.e. vascular endothelial cells.

The antibody may be prepared in any mammal; suitable mammals other than a human include, for example, a rabbit, rat, mouse, horse, goat, or primate. Mice are preferred. The antibody may be a member of one of the following immunoglobulin classes: IgG, IgM, IgA, IgD, or IgE, and the subclasses thereof, and preferably is an IgG1 antibody. The antibodies of the invention and their functional equivalents may be or may combine members of any of the immunoglobulin classes.

### Non-Antibody VEGFR Antagonists

In addition to the antibodies, or functional equivalents of antibodies, discussed above, the receptor antagonists useful in the present invention may also be other biological and small molecules, especially in connection with the treatments described above.

Biological molecules include all lipids and polymers of monosaccharides, amino acids and nucleotides having a molecular weight greater than 450. Thus, biological molecules include, for example, oligosaccharides and polysaccharides; oligopeptides, polypeptides, peptides, and proteins; and oligonucleotides and polynucleotides. Oligonucleotides and polynucleotides include, for example, DNA and RNA.

Biological molecules further include derivatives of any of the molecules described above. For example, derivatives of biological molecules include lipid and glycosylation derivatives of oligopeptides, polypeptides, peptides and proteins. Derivatives of biological molecules further include lipid derivatives of oligosaccharides and polysaccharides, for example, lipopolysaccharides.

Any molecule that is not a biological molecule is considered in this specification to be a small molecule. Small molecules of the present invention are entities having carbon and hydrogen atoms, as well as heteroatoms, which include, but are not limited to, nitrogen, sulfur, oxygen, and phosphorus. Atoms in a small molecule are linked together via covalent and ionic bonds; the former is typical for small organic compounds, e.g., small molecule tyrosine kinase inhibitors such as Iressa™ and Tarceva™ and the latter is typical of small inorganic compounds. The arrangement of atoms in a small organic molecule may represent a chain, e.g., a carbon-carbon chain or carbon-heteroatom chain, or ring containing carbon atoms, e.g., benzene, or a combination of carbon and heteroatoms, i.e., heterocycles, for example, a pyrimidine or quinazoline. A combination of one or more chains in a small organic molecule attached to a ring system constitutes a substituted ring system and fusion of two rings constitutes a fused policyclic system, which can be referred to as simply a policyclic system, an example of which is the parent scaffold of Iressa.™ Small molecules include both compounds found in nature, such as hormones, neurotransmitters, nucleotides, amino acids, sugars, lipids and their derivatives, and those compounds made synthetically, either by traditional organic synthesis, bio-mediated synthesis, or a combination thereof. See, e.g., Ganesan, Drug Discov. Today, 7(1): 47-55 (January 2002); Lou, Drug Discov. Today, 6(24): 1288-1294 (December 2001).

Some examples of small molecules include organic compounds, organometallic compounds, salts of organic and organometallic compounds, saccharides, amino acids, nucleosides and nucleotides. It is emphasized that small molecules can have any molecular weight. They are merely called small molecules because they typically have molecular weights less than 450. Small molecules include compounds that are found in nature as well as synthetic compounds.

The administration of small molecule and biological drugs to human patients is accomplished by methods known in the art. Examples of such methods for small molecules are described in Spada, U.S. Pat. No. 5,646,153 at column 57, line 47 to column 59, line 67. This description of administering small molecules is incorporated herein by reference.

### Neutralizing VEGF Activation of VEGF Receptors

Neutralization of activation of a VEGF receptor in a sample of endothelial or non-endothelial cells, such as tumor cells, may be performed in vitro or in vivo. Neutralizing VEGF activation of a VEGF receptor in a sample of VEGF-receptor expressing cells comprises contacting the cells with an antagonist, e.g., an antibody, of the invention. The cells are contacted in vitro with the antagonist, e.g., the antibody, before, simultaneously with, or after, adding VEGF to the cell sample.

In vivo, an antagonist, e.g., an antibody, of the invention is contacted with a VEGF receptor by administration to a mammal. Methods of administration to a mammal include, for example, oral, intravenous, intraperitoneal, subcutaneous, or intramuscular administration.

This in vivo neutralization method is useful for inhibiting angiogenesis in a mammal. Angiogenesis inhibition is a useful therapeutic method, such as for preventing or inhibiting angiogenesis associated with pathological conditions such as tumor growth. Accordingly, the antagonists, e.g., the antibodies, of the invention are anti-angiogenic and anti-tumor immunotherapeutic agents.

The word mammal means any mammal. Some examples of mammals include pet animals, such as dogs and cats; farm animals, such as pigs, cattle, sheep, and goats; laboratory animals, such as mice and rats; primates, such as monkeys, apes, and chimpanzees; and humans.

VEGF receptors are found on some non-endothelial cells, such as tumor cells, indicating the unexpected presence of an autocrine and/or paracrine loop in these cells. The antagonists, e.g., the antibodies, of this invention are useful in neutralizing activity of VEGF receptors on such cells, thereby blocking the autocrine and/or paracrine loop, and inhibiting tumor growth.

The methods of inhibiting angiogenesis and of inhibiting pathological conditions such as tumor growth in a mammal comprise administering an effective amount of any one of the invention's antagonists, e.g., antibodies, including any of the functional equivalents thereof, systemically to a mammal, or directly to a tumor within the mammal. The mammal is preferably human. This method is effective for treating subjects with both solid tumors, preferably highly vascular tumors, and non-solid tumors.

The inhibition or reduction of tumor growth includes the prevention or inhibition of the progression of a tumor, including cancerous and noncancerous tumors. The progression of a tumor includes the invasiveness, metastasis, recurrence and increase in size of the tumor. The inhibition or reduction of tumor growth also includes the destruction of a tumor.

All types of tumors may be treated by the methods of the present invention. The tumors may be solid or non-solid.

Some examples of solid tumors that can be treated with the antagonists of the present invention include carcinomas, sarcomas, blastomas or gliomas. Some examples of such tumors include epidermoid tumors, squamous tumors, such as head and neck tumors, colorectal tumors, prostate tumors, breast tumors, lung tumors, including small cell and non-small cell lung tumors, pancreatic tumors, thyroid tumors, ovarian tumors, and liver tumors. Other examples include Kaposi's sarcoma, CNS neoplasms, neuroblastomas, capillary hemangioblastomas, meningiomas and cerebral metastases, melanoma, gastrointestinal and renal carcinomas and sarcomas, rhabdomyosarcoma, glioblastoma, preferably glioblastoma multiforme, and leiomyosarcoma. Examples of vascularized skin cancers for which the antagonists of this invention are effective include squamous cell carcinoma, basal cell carcinoma and skin cancers that can be treated by suppressing the growth of malignant keratinocytes, such as human malignant keratinocytes.

Some examples of non-solid tumors include leukemias, multiple myelomas and lymphomas. Some examples of leukemias include acute myelocytic leukemia (AML), chronic myelocytic leukemia (CML), acute lymphocytic leukemia (ALL), chronic lymphocytic leukemia (CLL), erythrocytic leukemia or monocytic leukemia. Some examples of lymphomas include lymphomas associated with Hodgkin's disease and Non-Hodgkin's disease.

Experimental results described later demonstrate that antibodies of the invention specifically block VEGF induced phosphorylation of a mouse extracellular flk-1 (VEGFR-2) (VEGFR-2)/intracellular fms chimeric receptor expressed in transfected 3T3 cells. The antibodies had no effect on a fully stimulated chimeric extracellular fms/intracellular FLK-2 receptor by CSF-1. In vivo studies also described below show that the antibodies were able to significantly inhibit tumor growth in nude mice.

A cocktail of VEGF receptor antagonists, e.g., monoclonal antibodies, provides an especially efficient treatment for inhibiting the growth of tumor cells. The cocktail may include as few as 2, 3 or 4 antibodies, and as many as 6, 8 or 10 antibodies.

Preventing or inhibiting angiogenesis is also useful to treat non-neoplastic pathologic conditions characterized by excessive angiogenesis, such as neovascular glaucoma, proliferative retinopathy including proliferative diabetic retinopathy, arthritis, macular degeneration, hemangiomas, angiofibromas, and psoriasis.

### Using the Antagonists of the Invention to Isolate and Purify the VEGF Receptor

The antagonists of the present invention may be used to isolate and purify the VEGF receptor using conventional methods such as affinity chromatography (Dean et al., Affinity Chromatography: A Practical Approach, IRL Press, Arlington, Va. (1985)). Other methods well known in the art include magnetic separation with antibody-coated magnetic beads, “panning” with an antibody attached to a solid matrix, and flow cytometry.

The source of the VEGF receptor is typically vascular cells, and especially vascular endothelial cells, that express the VEGF receptor. Suitable sources of vascular endothelial cells are blood vessels, such as umbilical cord blood cells, especially, human umbilical cord vascular endothelial cells (HUVEC).

The VEGF receptors may be used as starting material to produce other materials, such as antigens for making additional monoclonal and polyclonal antibodies that recognize and bind to the VEGF receptor or other antigens on the surface of VEGF-expressing cells.

Using the Antagonists of the Invention to Isolate and Purify flk-1 (VEGFR-2) Positive Tumor Cells

The antagonists of the present invention may be used to isolate and purify flk-1 (VEGFR-2) (VEGFR-2) positive tumor cells, i.e., tumor cells expressing the flk-1 (VEGFR-2) receptor, using conventional methods such as affinity chromatography (Dean, P. D. G. et al., Affinity Chromatography: A Practical Approach, IRL Press, Arlington, Va. (1985)). Other methods well known in the art include magnetic separation with antibody-coated magnetic beads, cytotoxic agents, such as complement, conjugated to the antibody, “panning” with an antibody attached to a solid matrix, and flow cytometry.

### Monitoring Levels of VEGF and VEGF Receptors in Vitro or in Vivo

The antagonists, e.g., antibodies, of the invention may be used to monitor levels of VEGF or VEGF receptors in vitro or in vivo in biological samples using standard assays and methods known in the art. Some examples of biological samples include bodily fluids, such as blood. Standard assays involve, for example, labeling the antibodies and conducting standard immunoassays, such as radioimmunoassays, as is well know in the art.

Standard recombinant DNA techniques useful in carrying out the present invention are described in Sambrook et al., “Molecular Cloning,” Second Edition, Cold Spring Harbor Laboratory Press (1987) and by Ausubel et al. (Eds) “Current Protocols in Molecular Biology,” Green Publishing Associates/Wiley-Interscience, New York (1990).

### Administration

The present receptor antagonists can be administered for therapeutic treatments to a patient suffering from a tumor in an amount sufficient to prevent, inhibit, or reduce the progression of the tumor, e.g, the growth, invasiveness, metastases and/or recurrence of the tumor. An amount adequate to accomplish this is defined as a therapeutically effective close. Amounts effective for this use will depend upon the severity of the disease and the general state of the patient's own immune system. Dosing schedules will also vary with the disease state and status of the patient, and will typically range from a single bolus dosage or continuous infusion to multiple administrations per day (e.g., every 4-6 hours), or as indicated by the treating physician and the patient's condition. It should be noted, however, that the present invention is not limited to any particular dose.

The present invention can be used to treat any suitable tumor, including, for example, tumors of the breast, heart, lung, small intestine, colon, spleen, kidney, bladder, head and neck, ovary, prostate, brain, pancreas, skin, bone, bone marrow, blood, thymus, uterus, testicles, cervix or liver. Preferably, the present methods are used when the tumor is a tumor of the colon or when the tumor is a non-small cell lung carcinoma (NSCLC).

Moreover, the tumors of the present invention preferably overexpress EGFR. Enhanced expression of EGFR has been detected in a significant percentage of many human tumors; for example, head and neck (80-100%), colorectal (25-77%), pancreatic (30-50%), lung (40-80%), esophageal (43-89%), renal cell (50-90%), prostate (65%), bladder (31-48%), cervical/uterus (90%), ovarian (35-70%) and breast (14-91%). Expression of EGFR has also been demonstrated to be an indicator of poor prognosis, decreased survival, and increased metastases in certain tumor types.

Furthermore, the tumors of the present invention preferably have aberrant expression or signaling of VEGFR. Enhanced signaling by VEGFR has been observed in many different human cancers. High levels of VEGFR-2 are expressed by endothelial cells that infiltrate gliomas (Plate et al., (1992) Nature 359:845-848). VEGFR-2 levels are specifically upregulated by VEGF produced by human glioblastomas (Plate et al. (1993) Cancer Res. 53:5822-5827). The finding of high levels of VEGFR-2 expression in glioblastoma associated endothelial cells (GAEC) indicates that receptor activity is probably induced during tumor formation since VEGFR-2 transcripts are barely detectable in normal brain endothelial cells. This upregulation is confined to the vascular endothelial cells in close proximity to the tumor.

The present invention is useful for inhibition or reduction of tumor growth. By inhibition or reduction of tumor growth is meant prevention, inhibition, or reduction of the progression of the tumor, e.g, the growth, invasiveness, metastases and/or recurrence of the tumor. In addition, the present invention also can be useful in treating an angiogenic condition, such as atherosclerosis, arthritis, macular degeneration and psoriasis. The identification of those patients that have conditions for which the present invention is useful is well within the ability and knowledge of one skilled in the art.

In the present invention, any suitable method or route, can be used to administer the EGFR antagonist and/or the VEGFR antagonist, for example, oral, intravenous, intraperitoneal, subcutaneous, or intramuscular administration. The dose of antagonist administered depends on numerous factors, including, for example, the type of antagonists, the type and severity tumor being treated and the route of administration of the antagonists, it should be emphasized, however, that the present invention is not limited to any particular method or route of administration.

In one alternative embodiment, the EGFR antagonist and the VEGFR antagonist can be administered in combination with one or more antineoplastic agents. Sec, e.g., U.S. Pat. No. 6,217,866 (Schlessinger et al.) (Anti-EGFR antibodies in combination with antineoplastic agents); U.S. application Ser. No. 09/312,286, abandoned (Waksal et al.) (Anti-EGFR antibodies in combination with radiation). Any suitable antineoplastic agent can be used,

such as a chemotherapeutic agent or radiation. Examples of chemotherapeutic agents include, but are not limited to, cisplatin, doxorubicin, paclitaxel, irinotecan (CPT-11), topotecan or a combination thereof. When the antineoplastic agent is radiation, the source of the radiation can be either external (external beam radiation therapy—EBRT) or internal (brachytherapy—BT) to the patient being treated. The dose of antineoplastic agent administered depends on numerous factors, including, for example, the type of agent, the type and severity tumor being treated and the route of administration of the agent. It should be emphasized, however, that the present invention is not limited to any particular dose.

In an additional alternative embodiment, the EGFR antagonist and the VEGFR antagonist can be administered in combination with one or more suitable adjuvants, such as, for example, cytokines (IL-10 and IL-13, for example) or other immune stimulators. See, e.g., Larrivée et al., supra. It should be appreciated, however, that administration of only an EGFR antagonist and a VEGFR antagonist is sufficient to prevent, inhibit, or reduce the progression of the tumor in a therapeutically effective manner.

In addition, the EGFR antagonist and/or the VEGFR antagonist can be administered as a ligand conjugate, which binds specifically to the receptor and deliver a toxic, lethal payload following ligand-toxin internalization. Conjugates between toxins and the receptors were designed with the aim of developing toxic agents specific for EGFR- or VEGFR-overexpressing tumor cells while minimizing nonspecific toxicity. For example, a conjugate composed of EGF and Pseudomonas endotoxin (PE) was shown to be toxic toward EGFR-expressing HeLa cells in vitro. Various agents, including thioridazine and adenovirus, can enhance cellular uptake of the conjugate, as well as increase the cytotoxicity of the conjugate.

It is understood that the EGFR and/or VEGFR antagonists of the invention, where used in a mammal for the purpose of prophylaxis or treatment, will be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection may, as is well known in the art, be formulated so as to provide quick, sustained or delayed release of the active ingredient after administration to the mammal.

The EGFR antagonists and/or VEGFR antagonists of this invention may be in a variety of forms. These include, for example, solid, semi-solid and liquid dosage forms, such as tablets, pills, powders, liquid solutions, dispersions or suspensions, liposomes, suppositories, injectable and infusible solutions. The preferred form depends on the intended mode of administration and therapeutic application.

Such antagonists can be prepared in a manner well known in the pharmaceutical art. In making the composition the active ingredient will usually be mixed with a carrier, or diluted by a carrier, and/or enclosed within a carrier which may, for example, be in the form of a capsule, sachet, paper or other container. When the carrier serves as a diluent, it may be a solid, semi-solid, or liquid material, which acts as a vehicle, excipient or medium for the active ingredient. Thus, the composition may be in the form of tablets, lozenges, sachets, cachets, elixirs, suspensions, aerosols (as a solid or in a liquid medium), ointments containing for example up to 10% by weight of the active compound, soft and hard gelatin capsules, suppositories, injection solutions, suspensions, sterile packaged powders and as a topical patch.

### Kits for Inhibition of Tumor Growth

The present invention also includes kits for inhibiting tumor growth comprising a therapeutically effective amount of an EGFR antagonist and a therapeutically effective amount of a VEGFR antagonist. The EGFR or VEGFR antagonist of the present kits can be any suitable antagonist, examples have been described above. Preferably, the EGFR antagonist of the kit comprises an antibody, or functional equivalent thereof, specific for EGFR. Alternatively, and also preferably, the EGFR antagonist of the kit comprises a small molecule specific for EGFR. The VEGFR antagonist of the kit preferably comprises an antibody, or functional equivalent thereof, specific for VEGFR. Alternatively, the VEGFR antagonist of the kit preferably comprises a small molecule specific for VEGFR. In addition, the kits of the present invention can further comprise an antineoplastic agent. Examples of suitable antineoplastic agents in the context of the present invention have been described herein. The kits of the present invention can further comprise an adjuvant, examples have also been described above.

Accordingly, the present receptor antagonists thus can be used in vivo and in vitro for investigative, diagnostic, prophylactic, or treatment methods, which are well known in the art. Of course, it is to be understood and expected that variations in the principles of invention herein disclosed can be made by one skilled in the art and it is intended that such modifications are to be included within the scope of the present invention.

All references mentioned herein are incorporated in their entirety.

### EXAMPLES

The Examples that follow are set forth to aid in understanding the invention but are not intended to, and should not be construed to, limit its scope in any way. The Examples do not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989) Molecular Cloning: A Laboratory Manual, 2nd edition, Cold Spring Harbor Laboratory Press.

### Example I

### Cell Lines and Media

NIH 3T3 cells were obtained from the American Type Culture Collection (Rockville Md.). The C441 cell line was constructed by transfecting 3T3 cells with the chimeric receptor mouse flk-1 (VEGFR-2)/human fms. 10A2 is a 3T3 transfectant containing the chimeric receptor human fms/mouse FLK-2, the isolation and characterization of which has been described (Dosil et al., Mol. Cell. Biol. 13:6572-6585 (1993)). Cells were routinely maintained in Dulbecco's modified Eagle's medium (DME) supplemented with 10% calf serum (CS), 1 mM L-glutamine, antibiotics, and 600 ug/ml G418 (Geneticin; Sigma, St Louis Mo.).

A glioblastoma cell line, GBM-18, was maintained in DME supplemented with 5% calf serum, 1 mM L-glutamine, and antibiotics.

A stable 3T3 line secreting the soluble chimeric protein, mouse flk-1 (VEGFR-2):SEAPs (secretory alkaline phosphastase), was generated and maintained in DMEM and 10% calf serum. Conditioned media was collected. Soluble flk-1 (VEGFR-2):SEAP is isolated from the conditioned media.

### Example II

### Isolation of Monoclonal Antibodies

### Example II-1

### Rat Anti Mouse flk-1 (VEGFR-2) Monoclonal Antibody DC-101 (IgG1)

Lewis rats (Charles River Labs) were hyperimmunized with an immune complex consisting of the mouse flk-1:SEAPs soluble receptor, a rabbit anti-alkaline phosphatase polyclonal antibody and Protein-G sepharose beads. The animals received 7 intraperitoneal injections of this complex spread over three months (at days 0, 14, 21, 28, 49, 63, 77). At various times, the animals were bled from the tail vein and immune sera screened by ELISA for high titer binding to mflk-1 (VEGFR-2): SEAPs. Five days after the final injection, rats were sacrificed and the spleens aseptically removed. Splenocytes were washed, counted, and fused at a 2:1 ratio with the murine myeloma cell line NS1. Hybridomas were selected in FIAT medium and colonies screened by ELISA for specific binding to mflk-1 (VEGFR-2): SEAPs but not the SEAPs protein. A number of positive hybridomas were expanded and cloned three times by limiting dilution. One subclone, designated DC-101, was further characterized.

### Example II-2

### Mouse Anti Mouse flk-1 (VEGFR-2) Monoclonal Antibodies Mab 25 and Mab 73

Murine anti-flk-1 (VEGFR-2) monoclonal antibodies (Mabs) were produced using a similar protocol as that employed for DC-101. Briefly, mice were injected with a complex of flk-1 (VEGFR-2)/SEAP soluble receptor bound to either an anti-SEAP-Protein/A Sepharose complex or wheat germ agglutinin Sepharose from conditioned medium of transfected NIH 3T3 cell. Mice were hyperimmunized at periodic intervals over a 6 month period. Immune splenocytes were pooled and fused with the murine myeloma cell line, NSL Hybridomas were selected in HAT medium and following incubation, colonies were screened for mouse Mab production. Unlike the protocol employed for DC-101, positive supernatants were initially screened for binding to the flk-1 (VEGFR-2)/fms receptor captured from C441 cell lysates on ELISA plates coated with a peptide generated polyclonal antibody against the C-terminal region of fms. Reactive Mabs were then assayed by ELISA for binding to intact C441 cells and to purified flk-1 (VEGFR-2)/SEAP versus SEAP alone. The supernatants from hybridomas showing binding to C441 and reactivity with flk-1 (VEGFR-2)/SEAP but not SEAP were expanded, grown in ascites, and purified (EZ-PREP, Pharmacia). Purified Mabs were subjected to assays on C441 cells to determine their cell surface binding by FACS and their ability to inhibit VEGF induced activation of flk-1 (VEGFR-2)/fms in phosphorylation assays. The results of these studies led to the cloning of Mabs 25 and 73 (isotype IgG1) for further characterization based on their capabilities to bind specifically to flk-1 (VEGFR-2) and block receptor activation at levels comparable to that observed for DC-101.

### Example III

### Assays

### Example III-1

### ELISA Methods

Antibodies were screened by a solid state ELISA in which the binding characteristics of the various mAbs to flk-1 (VEGFR-2):SEAP and SEAP protein were compared. Microliter plates were coated with 50-100 ng/well of either flk-1:SEAP or AP in pH9.6 carbonate buffer overnight at 4° C. Plates were blocked with phosphate buffered saline supplemented with 10% new born calf serum (NB10) for one hour at 37° C. Hybridoma supernatants or purified antibodies were added to the plates for two hours at 37° C. followed by goat anti-rat IgG conjugated to horseradish peroxidase (Tago) added for an additional hour at 37° C. After extensive washing, TMB (Kirkegaard and Perry, Gaithersburg Md.) plus hydrogen peroxide was added as the chromogen and the plates read at 450 nm in an ELISA reader.

### Example III-2

### Isotyping

Isotyping of the various monoclonal antibodies was done as previously described (Songsakphisam, R. and Goldstein, N. I., Hybridoma 12: 343-348, 1993) using rat isotype specific reagents (Zymed Labs, South San Francisco Calif.).

### Example III-3

### Phosphorylation, Immunoprecipitation and Immunoblot Assays

The phosphorylation assays and Western blot analysis with C441 and 30A2 cells were performed as previously described (Tessler et al., 1994) with some modifications. Briefly, cells were grown to 90% confluency in DME-10% CS and then serum starved in DME-0.5% CS for 24 hours prior to experimentation. HUVEC cells were grown to subconfluence in EGM basal media. For neutralization assays, cells were stimulated with various concentrations of the appropriate ligand under serum free conditions (DME −0.1% BSA) in the presence and absence of mAb DC-101 for 15 minutes at room temperature. The ligands, VEGF and CSF-1, were assayed at concentrations of 10-80 ng/ml and 20-40 ng/ml, respectively. Monoclonal antibodies were assayed at concentrations ranging from 0.5 μg/ml to 10 μg/ml. To evaluate the effects of mAb DC-101 on the VEGF induced activation of the flk-1 (VEGFR-2)-fms receptor, antibody was either added simultaneously (competitive inhibition) or prebound to cells for 15 minutes at room temperature prior to the addition of ligand. Cells incubated in serum free medium in the absence and presence of DC-101 served as controls for receptor autophosphorylation in the absence of ligand and the presence of antibody, respectively. A control cell line expressing the tins/FLK-2 chimeric receptor (10A2) was starved and stimulated with 20 and 40 ng/ml CSF-1 and assayed in the presence and absence of 5 μg/ml DC-101.

Following stimulation, monolayers were washed with ice cold PBS containing 1 mM sodium orthovanadate. Cells were then lysed in lysis buffer (20 mM Tris-HCl, pH 7.4, 1% Triton X-100, 137 mM NaCl, 10% glycerol, 10 mM EDTA, 2 mM sodium orthovanadate, 100 mM NaF, 100 mM sodium pyrophosphate, 5 mM Pefabloc (Boehringer Mannheim Biochemicals, Indianapolis Ind.), 100 μg aprotinin and 100 μg/ml leupeptin) and centrifuged at 14000×g for 10 minutes. Protein was immunoprecipitated from cleared lysates of transfected cells using polyclonal antibodies generated to peptides corresponding to the C-terminal region of the human fms receptor (Tessler et al., J. Biol. Chem. 269, 12456-12461, 1994) or the murine FLK-2 interkinase domain (Small et al., Proc. Nat'l Acad. Sci. USA, 91, 459-463, 1994) coupled to Protein A Sepharose beads. Where indicated, immunoprecipitations with DC-101 or irrelevant rat IgG were performed with 10 μg of antibody coupled to Protein G beads. The beads were then washed once with 0.2% Triton X-100, 10 mM Tris-HCl pH8.0, 150 mM NaCl, 2 mM EDTA (Buffer A), twice with Buffer A containing 500 mM NaCl and twice with Tris-HCl, pH 8.0. Drained beads were mixed with 30 μl in 2×SDS loading buffer and subjected to SDS PAGE in 4-12% gradient gels (Novex, San Diego Calif.). After electrophoresis, proteins were blotted to nitrocellulose filters for analysis. Filters were blocked overnight in blocking buffer (50 mM Tris-HCl, pH7.4, 150 mM NaCl (TBS) containing 5% bovine serum albumin and 10% nonfat dried milk (Biorad, CA). To detect phosphorylated receptor, blots were probed with a monoclonal antibody directed to phosphotyrosine (UBI, Lake Placid, N.Y.) in blocking buffer for 1 hour at room temperature. Blots were then washed extensively with 0.5×TBS containing 0.1% Tween-20 (TBS-T) and incubated with goat anti-mouse Ig conjugated to horseradish peroxidase (Amersham). Blots were washed with TBS and incubated for 1 minute with a chemiluminescence reagent (ECL, Amersham). Anti-phosphotyrosine reacting with phosphorylated proteins was detected by exposure to a high performance luminescence detection film (Hyperfilm-ECL, Amersham) for 0.5 to 10 minutes.

To detect flk-1 (VEGFR-2)/fms in C441 cells receptor levels, blots were stripped according to manufacturer's protocols (Amersham) and reprobed with the anti-fms rabbit polyclonal antibody.

### Example III-4

### Flow Cytometer Binding Assays

C441 cells were grown to near confluency in 10 cm plates. Cells were removed with a non-enzymatic dissociation buffer (Sigma), washed in cold serum free medium and resuspended in Hanks balanced salt solution supplemented with 1% BSA (HBSS-BSA) at a concentration of 1 million cells per tube. Monoclonal Ab DC-101 or an isotype matched irrelevant antibody anti FLK-2 23H7 was added at 10 μg per tube for 60 minutes on ice. After washing, 5 μl of goat anti-mouse IgG conjugated to FITC (TAGO) was added for an additional 30 minutes on ice. Cells were washed three times, resuspended in 1 ml of HBSS-BSA, and analyzed on a Coulter Epics Elite Cytometer. Non-specific binding of the fluorescent secondary antibody was determined from samples lacking the primary antibody.

### Example III-5

### Binding Assays to Intact Cells

Assays with C441 cells were performed with cells grown to confluency in 24 well dishes. HUVEC cells were grown to confluency in 6 well dishes. Monolayers were incubated at 4° C. for 2 hours with various amounts of mAb DC-101 in binding buffer (DMEM, 50 Mm HEPES pH 7.0, 0.5% bovine serum albumin). Cells were then washed with cold phosphate buffered saline (PBS) and incubated with a secondary anti-rat IgG antibody conjugated with biotin at a final concentration of 2.5 ug/ml. After 1 hour at 4° C. cells were washed and incubated with a streptavidin-horse radish peroxidase complex for 30 minutes at 4° C. Following washing, cell-bound antibody was determined by measuring the absorbance at 540 nm obtained with a colormetric detection system (TMB, Kirkegaard and Perry). The OD 540 nm of the secondary antibody alone served as the control for non-specific binding.

### Example III-6

### Cell Proliferation Assays

Mitogenic assays were performed using the Cell Titer 96 Non Radioactive Cell Proliferation Assay Kit (Promega Corp., Madison, Wis.). In this assay proliferation is measured color metrically as the value obtained from the reduction of a tetrazolium salt by viable cells to a formazan product. Briefly, HUVEC cells were grown in 24 well gelatin-coated plates in EGM basal media at 1000 cells/well. After a 48-hour incubation various components were added to the wells. VEGF was added at 10 ng/ml to the media in the presence and absence of 1 μg/ml of mAb DC-101. Where indicated, heparin (Sigma) was added to a final concentration of 1 μg/ml. Cells were then incubated for an additional 3 days. To measure cell growth, a 20 μl aliquot of tetrazolum dye was added to each well and cells were incubated for 3 hrs at 37° C. Cells were solubilized and the absorbance (OD570) of the formazan product was measured as a quantitation of proliferation.

### Example IV

### In Vitro Activity Assays

### Example IV-1

### Murine Anti-flk-1 (VEGFR-2) Mabs 25 and 73 Elicit a Specific Neutralization of VEGF Induced Activation of the flk-1 (VEGFR-2)/fms Receptor

Assays were performed with immunoprecipitated FLK/fms and PDGF receptors from equal concentrations of the flk-1 (VEGFR-2)/fms transfected 3T3 cell line, C441 whereas the human EGFR was immunoprecipitated from the tumor cell line, KB. Cells were stimulated with RPMI-0.5% BSA containing 20 ng/ml VEGF (flk-1/fms), DMEM-10% calf serum (PDGFR), or 10 ng/ml EGF (EGFR), in the presence and absence of 10 ug/ml of the murine anti-flk-1 Mabs, 25 and 73. Following stimulation, cells were washed with PBS-1 mM sodium orthovanadate and lysed. Flk-1/fms and PDGFR were immunoprecipitated from lysates with peptide generated polyclonal antibodies against the C-terminal region of the c-fms (IM 133) and the PDGF (UBI) receptors, respectively. EGFR was immunoprecipitated with a Mab (C225) raised against the N-terminal region of the human receptor. Immunoprecipitated lysates were subjected to SDS polyacrylamide electrophoresis followed by western blotting. Blots were probed with an anti-PTyr Mab (UBI) to detect receptor activation. Receptor neutralization of stimulated cells was assessed relative to an irrelevant Mab and the unstimulated control.

### Example IV-2

### Detection of the flk-1 (VEGFR-2)/fms Receptor by Western Blotting Using Mab 25 and Mab 73 as Probes

Receptor was detected by the murine anti-flk-1 (VEGFR-2) Mabs on western blots of the flk-1 (VEGFR-2)/fms receptor immunoprecipitated by a peptide generated polyclonal antibody against the C-terminal region of the c-fms receptor from lysated prepared from equal concentrations of transfected 3T3 cell line C441. Following analysis by SDS gel electrophoresis and western blotting, the blot was divided into four parts and each section was probed with 50 μg/ml of the anti-flk-1 (VEGFR-2) Mabs 25 and 73. Blots were then stripped and reprobed with the anti-fms polyclonal antibody to verify that the bands detected by each Mab represented the flk-1 (VEGFR-2)/fms receptor.

### Example IV-3

### Detection of Activated KDR (VEGFR-2) from VEGF Stimulated HUVEC and OVCAR-3 Cells by Immunoprecipitation with Anti-flk-1 (VEGFR-2) Mabs

Proteins were immunoprecipitated by different antibodies from a lysate of freshly isolated HUVEC. Prior to lysis, cells were stimulated with 20 ng/ml VEGF for 10 minutes at room temperature in RPMI-0.5% BSA and washed with PBS containing 1 mM sodium orthovanadate. Individual immunoprecipitations were performed with equal volumes of lysate and then subjected to SDS polyacrylamide electrophoresis followed by western blotting. The blot was probed initially with an anti-PTyr Mab (UBI) and then sequentially stripped and reprobed with a peptide generated polyclonal antibody against the interkinase of flk-1/KDR (VEGFR-1/VEGFR-2, IM 142), followed by a polyclonal antibody against the C-terminal region of flk-1 (VEGFR-1) (Santa Cruz Biotechnology, Inc). The immunoprecipitations were performed with an irrelevant rat Mab, 23H7, an irrelevant mouse Mab, DAB 8, versus the anti-flk-1 (VEGFR-2) Mabs, DC-101, 73, 25 and an anti-flk-1/KDR (VEGFR-1/VEGFR-2) polyclonal antibody, IM 142. In some cases blots were stripped and reprobed with the anti-flk-1 Mabs 73 and 25 to detect cross reactive bands.

A similar protocol was employed to detect KDR (VEGFR-2) receptor form(s) in the ovarian carcinoma cell line OVCAR-3.

### Example V

### Activity of Antibodies

### Example V-1

### ELISA and Immunoprecipitation with DC-101

Rat IgG1 monoclonal antibody DC-101 was found to be specific for the murine tyrosine kinase receptor flk-1 (VEGFR-2). ELISA data showed that the antibody bound to purified flk-1 (VEGFR-2):SEAP but not alkaline phosphatase or other receptor tyrosine kinases such as FLK-2. As seen in FIG. 1, DC-101 immunoprecipitates murine flk-1 (VEGFR-2): SEAPS but not SEAPS alone.

### Example V-2

### Inhibition Of Flk-1 (VEGFR-2) Receptor Phosphorylation with DC-101

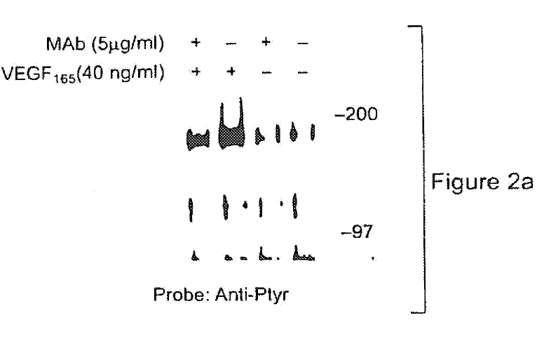

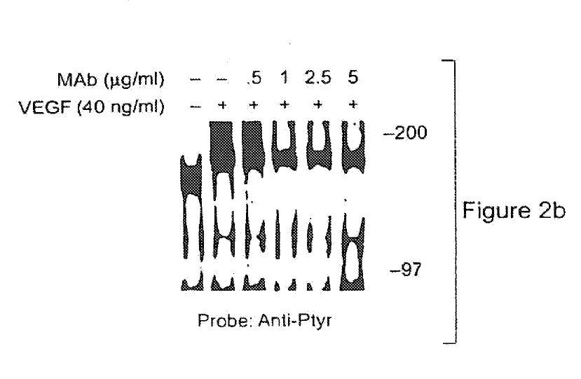

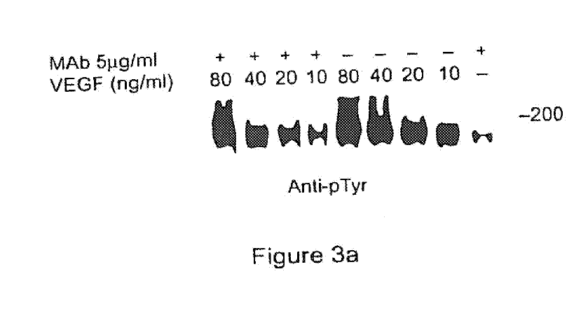

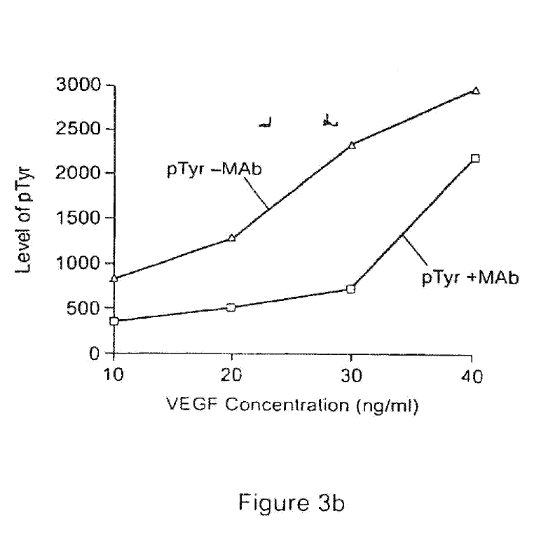

Experiments were then performed to determine whether DC-101 could neutralize phosphorylation of flk-1 (VEGFR-2) in C441 cells by its cognate ligand, VEGF₁₆₅. In these studies, monoclonal antibody and VEGF were added simultaneously to monolayers for 15 minutes at room temperature. These conditions were designed to determine the competitive effects (competitive inhibition) of the antibody on receptor/ligand binding. The results of these assays, shown in FIG. 2a, indicate that VEGF₁₆₅ induced phosphorylation of the flk-1 (VEGFR-2)/fms receptor was markedly reduced when cells were assayed in the presence of DC-101. In addition, these data suggest that the Mab competes with VEGF₁₆₅ to prevent a full activation of receptor by ligand. To determine the sensitivity of the VEGF-flk-1 (VEGFR-2) interaction to inhibition by DC-101, C441 cells were assayed at maximal stimulatory concentrations of VEGF₁₆₅ (40 ng/ml) combined with varying levels of the antibody. The results of these Mab titrations are shown in FIG. 2b. A marked decrease in the phosphorylation of flk-1 (VEGFR-2) by VEGF₁₆₅ was observed when DC-101 was included at concentrations greater than 0.5 μg/ml. These data show that relatively low concentrations of antibody (<1 μg/ml) are sufficient to inhibit receptor activation by ligand. At 5 μg/ml the antibody is able to neutralize VEGF₁₆₅ stimulation of flk-1 (VEGFR-2) in the presence of excess ligand at 80 ng/ml (FIGS. 3a and 3b). As a control, the effect of DC-101 was tested on the fully stimulated fms/FLK-2 receptor (30A2 cell line) using CSF-1. Under these conditions, DC-101 showed no effect on receptor activation.

### Example V-3

### Inhibition Studies with DC-101

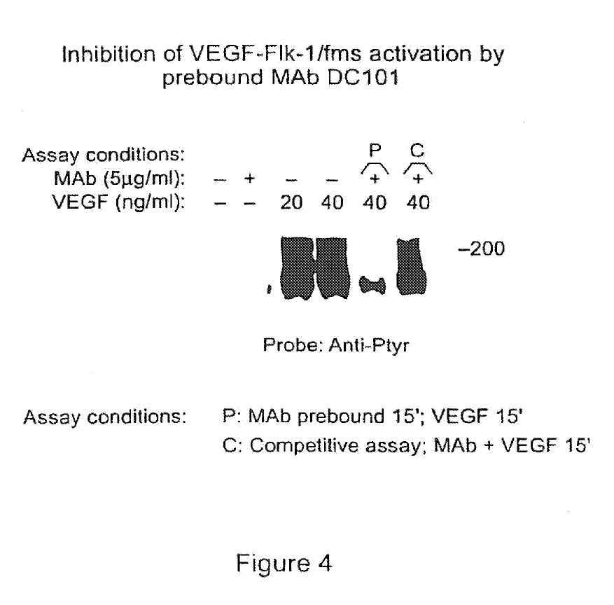

The extent and specificity of Mab inhibition was further assessed by studies in which DC-101 was preincubated with cells before the addition of ligand to allow maximal interaction of antibody with receptor. In these experiments, monolayers were incubated with 5 μg/ml of DC-101, a rat anti-FLK-2 Mab (2A13) prepared by conventional techniques (ImClone, NY), and control rat IgG1 (Zymed Labs) for 15 minutes at room temperature prior to the addition of 40 ng/ml of VEGFRs for an additional 15 minutes. For comparison, assays were run in which DC-101 and VEGFRs were added simultaneously (competitive inhibition). The results of these studies (FIG. 4) show that preincubation of the anti-flk-1 (VEGFR-2) monoclonal antibody with flk-1 (VEGFR-2)/fms transfected cells completely abrogates receptor activation by VEGF₁₆₅. Similar results were observed using VEGF₁₂₁ for stimulation. While phosphorylation of flk-1 (VEGFR-2) by VEGF is not affected by the addition of irrelevant isotype matched rat antibodies, the reactivity of the same blot probed with the anti-fms polyclonal antibody shows an equal level of receptor protein per lane. These data indicate that the inhibition of phosphorylation observed with DC-101 was due to the blockage of receptor activation rather than a lack of receptor protein in the test samples.

### Example V-4

### Binding of DC-101 to C441 cells by FACS Analysis

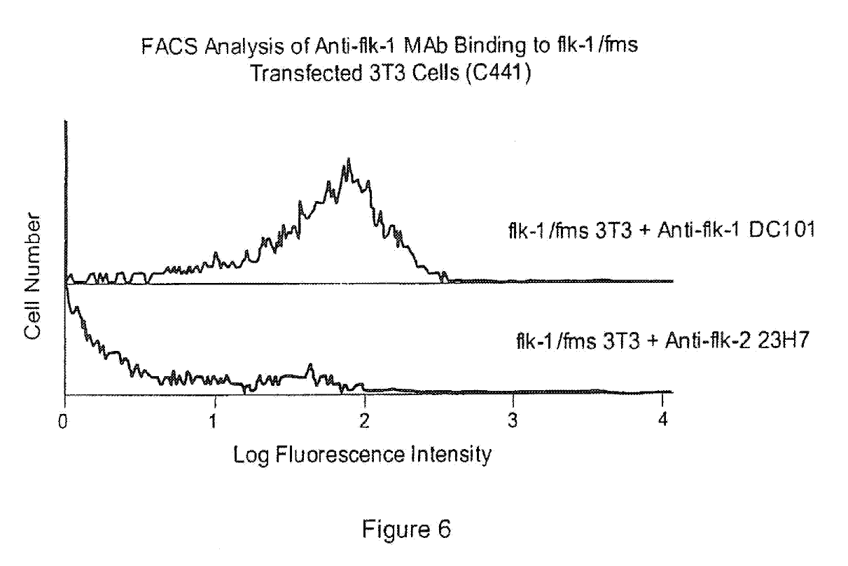

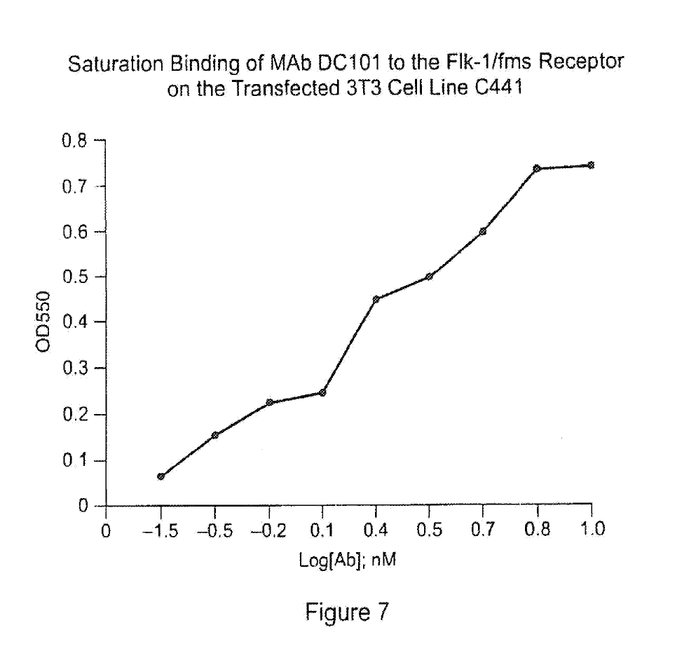

The mAb was assayed by FACS analysis for binding to 3T3 cells transfected with the flk-1 (VEGFR-2)/fms receptor (C441 cells). The results, shown in FIG. 6, demonstrate that the chimeric flk-1 (VEGFR-2)/fms expressed on the surface of C441 cells is specifically recognized by mAb DC-101 and not by an antibody of the same isotype raised against the related tyrosine kinase receptor, FLK-2. The efficacy of the mAb-receptor interaction at the cell surface was determined from assays in which varying levels of mAb binding was measured on intact C441 cells. These results, shown in FIG. 7, indicate that mAb binds to the flk-1 (VEGFR-2)/fms receptor with a relative apparent affinity of approximately 500 ng/ml. These results indicate that the mAb has a strong affinity for cell surface expressed flk-1 (VEGFR-2).

### Example V-5

### Reactivity of DC-101 by Immunoprecipitation

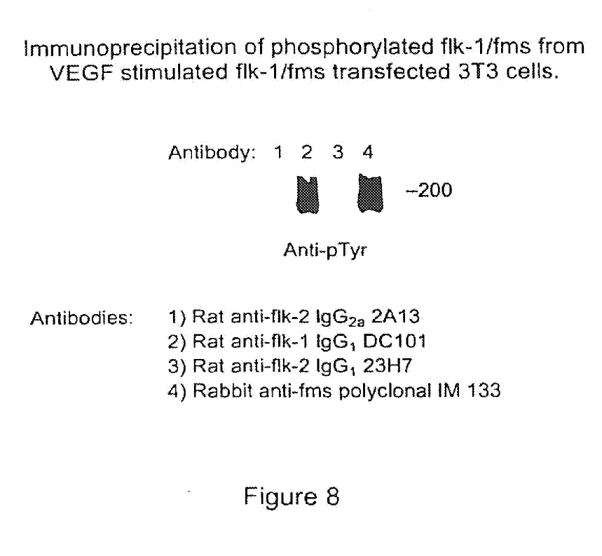

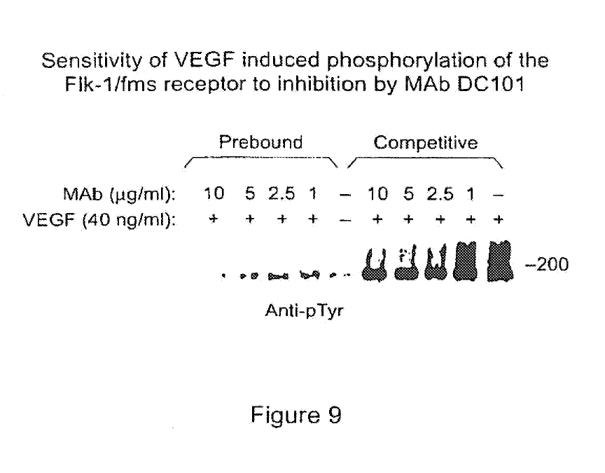

The extent of DC-101 reactivity with the flk-1 (VEGFR-2)/fms receptor was further assessed by determining the capacity of the antibody to immunoprecipitate the receptor following activation by VEGF. FIG. 8 shows an immunoprecipitation by mAb DC-101 of the phosphorylated flk-1 (VEGFR-2)/fms receptor from VEGF stimulated C441 cells. The results show that the DC-101 monoclonal and anti-fms polyclonal antibodies display similar levels of receptor interaction while rat anti-FLK-2 antibodies 2H37 (IgG1) and 2A13 (IgG2a) show no reactivity. Experiments were then performed to determine whether mAb DC-101 could neutralize the VEGF induced phosphorylation of flk-1 (VEGFR-2)/fms at maximal stimulatory concentrations of ligand (40 ng/ml). In these studies, monoclonal antibody was added to monolayers either simultaneously with ligand or prior to ligand stimulation and assayed for 15 minutes at room temperature. These conditions were studied to determine both the competitive effects (competitive inhibition) of the antibody on receptor/ligand binding as well as the efficacy of prebound antibody to prevent receptor activation. The results of these assays, shown in FIG. 4, indicate that phosphorylation of the flk-1 (VEGFR-2)/fms is reduced by the simultaneous addition of mAb with VEGF and markedly inhibited by antibody prebound to the receptor. A densitometry scan of these data revealed that mAb DC-101 interacts with flk-1 (VEGFR-2)/fms to inhibit phosphorylation to a level that is 6% (lane 5, P) and 40% (lane 6, C) of the fully stimulated receptor control (lane 4). From these data we infer that mAb DC-101 strongly competes with the ligand-receptor interaction to neutralize flk-1 receptor activation. To determine the sensitivity of the VEGF-flk-1 (VEGFR-2) interaction to inhibition by mAb DC-101, C441 cells were assayed with maximal VEGF levels in the presence of increasing concentrations of antibody. Assays were performed with the mAb under competitive and prebinding conditions. The results of these mAb titrations are shown in FIG. 9. A marked decrease in the phosphorylation of flk-1 (VEGFR-2) is observed when mAb DC-101 competes with VEGF antibody at concentrations greater than 0.5 μg/ml. These data also show that relatively low concentrations of prebound antibody (<1 μg/ml) are sufficient to completely inhibit receptor activation by ligand.

### Example V-6

### Activity of DC-101 by Phosphorylation Assay

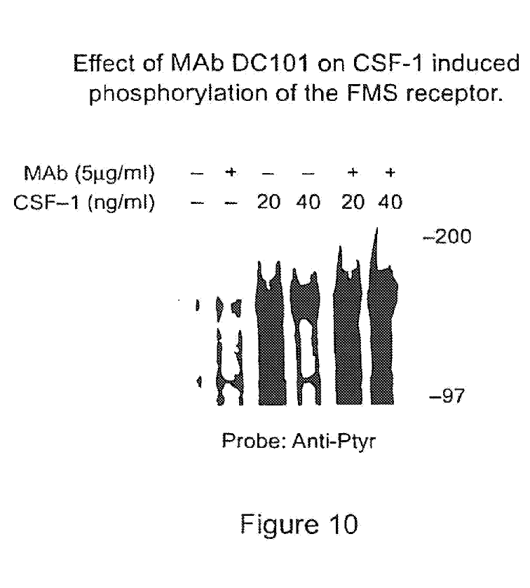

To further evaluate the antagonistic behavior of mAb DC-101 on receptor activation, phosphorylation assays were performed in which a fixed amount of antibody (5 μg/ml) was added to C441 cells stimulated with increasing amounts of ligand (FIG. 3a). The level of phosphorylation induced by each ligand concentration in the presence and absence of mAb DC-101 was also quantitated by densitometry readings. The plot of these data given in FIG. 3b indicates that the antibody was able to partially neutralize receptor phosphorylation even in the presence of excess amounts of VEGF. To evaluate the specificity of mAb DC-101 on receptor activation, the antibody was tested for its ability to competitively inhibit CSF-1 induced activation of the fms/FLK-2 receptor in the 3T3 transfected cell line, 10A2. In these experiments 5 μg/ml of mAb DC-101 was tested together with CSF-1 concentrations (20-40 ng/ml) that are known to result in full activation of the receptor. These results, which are shown in FIG. 10, indicate that mAb DC-101 has no effect on the CSF-1 mediated phosphorylation of the fms/FLK-2 receptor.

### Example V-7

### DC-101 Inhibition by Pre-Incubation Studies

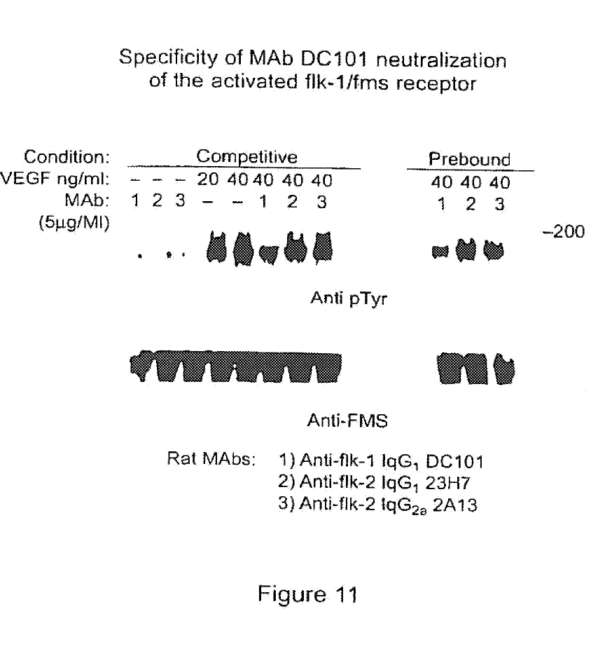

The extent and specificity of antibody inhibition was further assessed by studies in which DC-101 or an irrelevant antibodies were preincubated with cells before the addition of ligand to assure maximal interaction of antibody with receptor. In these experiments, monolayers were preincubated with either 5 μg/ml of DC-101, a rat anti-FLK-2 mAb (2A13) or a control rat IgG1 (Zymed Labs) prior to the addition of 40 ng/ml of VEGF. For comparison, competitive assays were run in which antibodies and VEGF were added simultaneously. The results of these studies show that only the preincubation of the anti-flk-1 (VEGFR-2) monoclonal antibody with flk-1 (VEGFR-2)/fms transfected cells completely abrogates receptor activation by VEGF while phosphorylation of flk-1 (VEGFR-2) by VEGF is not affected by the addition of irrelevant isotype matched rat antibodies. The reactivity of the same blot probed with the anti-fins polyclonal (FIG. 11) shows an equal level of receptor protein per lane. These data indicate that the lack of phosphorylation observed with mAb DC-101 treated cells was due to the blockage of a VEGF-induced phosphorylation of equal amounts of expressed receptor.

### Example V-8

### Interaction of Antibodies with Homologous Receptor Forms

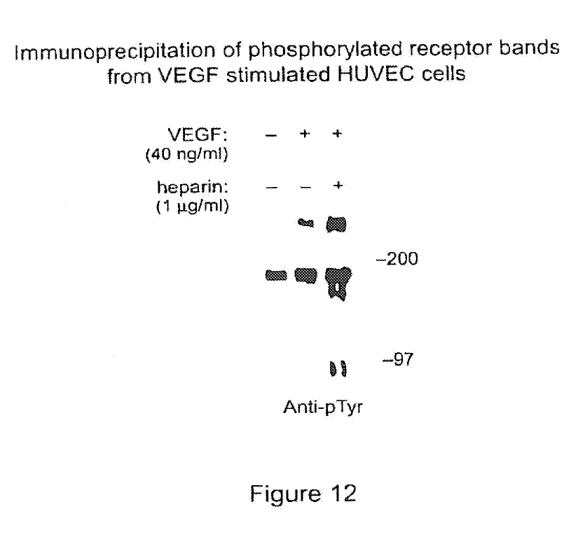

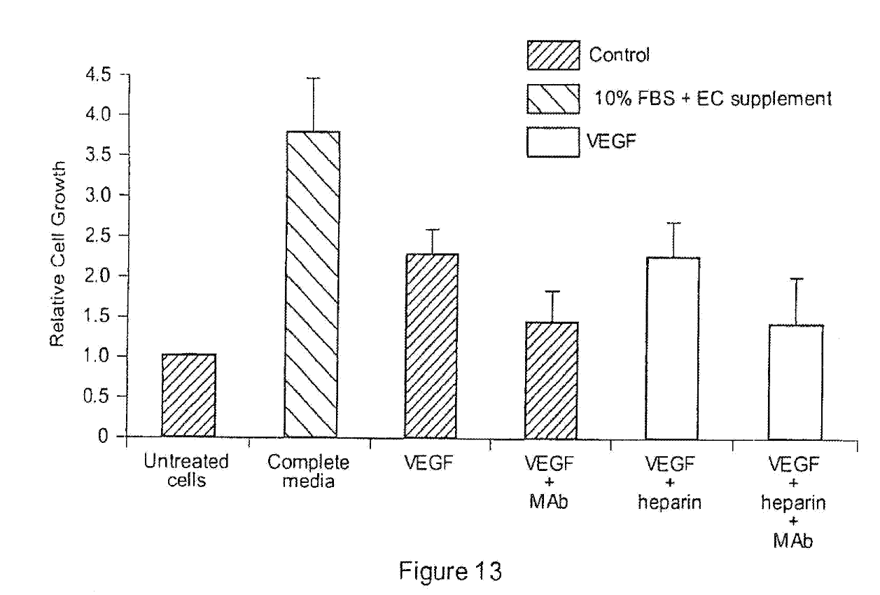

Experiments were then conducted to determine whether the anti-flk-1 (VEGFR-2) monoclonal antibodies interact with homologous receptor forms on human endothelial cells. A titration of increasing concentrations of DC-101 on cloned HUVEC cells (ATCC) indicated that the antibody displayed a complex binding behavior. The data represent differential antibody interactions with VEGF receptors reported to occur on endothelial cells (Vaisman et al., J. Biol. Chem. 265, 19461-19466, 1990). The specificity of DC-101 interaction with VEGF stimulated HUVEC cells was then addressed using phosphorylation assays under similar conditions as those reported for FIG. 8. In these studies DC-101 immunoprecipitates protein bands from HUVEC cells that have molecular weights similar to those reported for cross linked VEGF-receptor bands when the ligand component is subtracted (FIG. 12). These bands display an increased phosphorylation when cells are stimulated by VEGF (compare lanes 1 and 2 in FIG. 12). In addition, the VEGF induced phosphorylation of the receptor bands is potentiated by the inclusion of 1 μg/ml heparin in the assay (lane 3 in FIG. 12). These findings are consistent with previous reports of increased VEGF binding to endothelial cells in the presence of low concentrations of heparin (Gitay-Goren et al., J. Biol. Chem. 267, 6093-6098.1992).

It is difficult to ascertain which immunoprecipitated protein interacts with DC-101 to generate the complex of phosphorylated bands observed in FIG. 12 given the various receptor forms shown to bind VEGF on HUVEC and the possibility of their association upon stimulation. Cell surface expressed receptor forms with molecular weights of approximately 180 (KDR (VEGFR-2)), 155, 130-135, 120-125 and 85 have been reported to bind VEGF on HUVEC. Such findings address the possibility that several different receptor forms may heterodimerize upon ligand stimulation in a manner similar to that reported for KDR-flk-1 (VEGFR-2/VEFGR-1). However, with the exception of KDR (VEGFR-2), the exact nature and role of these receptor forms have yet to be defined. Consequently, antibody reactivity may result from interaction(s) with one of several VEGF receptors independent of KDR (VEGFR-2).

DC-101 does not react with human KDR (VEGFR-2) in an ELISA format nor bind to freshly isolated HUVEC by FACS analysis. These results suggest that a direct interaction of DC-101 with human KDR (VEGFR-2) is highly unlikely.

Unlike DC-101, Mab 25 and Mab 73 both react with human KDR (VEGFR-2) in an ELISA format and bind to freshly isolated HUVEC by FACS analysis.

### Example V-9

### Mitogenic Assays of HUVEC

An inhibitory effect of DC-101 on endothelial cells was observed when the antibody was tested in mitogenic assays of HUVEC cells (ATCC) stimulated with VEGF in the presence and absence of antibody (FIG. 12). These results show that a marked increase in cell proliferation by VEGF is reduced approximately 35% by DC-101. Heparin shows no differential effect on cell growth under the growth conditions employed in these assays.

Since DC-101 can exert effects on VEGF induced proliferation and receptor phosphorylation of HUVEC it is conceivable that these results are due to a Mab interaction with an undefined receptor form which is poorly accessible at the cell surface, but which plays some role, albeit minor, in HUVEC growth. Also, the immunoprecipitation of phosphorylated bands of the correct molecular weight by DC-101 from VEGF stimulated HUVEC also supports the notion that DC-101 may interact with an undefined flk-1 (VEGFR-2) like protein that associates with an activated receptor complex.

### Example V-10

### Binding of Mab 25 and Mab 73 to C441 Cells and HUVEC

Mabs 25 and 73 bind to C441 and HUVEC by FACS analysis and show internalization in both cell lines. Results from western blots show that both anti-flk-1 Mabs can detect the band(s) for the FLK/fms receptor in immunoprecipitates by an anti-fms polyclonal antibody from C441 cells. (See example IV-2 above for protocol.) These antibodies elicit a specific neutralization of VEGF induced activation of the flk-1 (VEGFR-2)/fms receptor and have no effect on the phosphorylation of the mouse PDGF receptor by PDGF or the human EGF receptor by EGF. (See example IV-1 above for protocol.) They have the capacity to inhibit VEGF stimulated HUVEC in proliferation assays to 50% whereas DC-101 affects growth to a far lesser extent.

### Example V-11

### Immunoprecipitation of KDR (VEGFR-2) with Mab25 and Mab 73

KDR (VEGFR-2) represents one of the phosphoproteins immunoprecipitated by the Mab25 and Mab 73 from activated HUVEC. KDR (VEGFR-2) was detected in western blot and immunoprecipitation analyses using an anti-flk-1/KDR (VEGFR-1/VEGFR-2) polyclonal antibody (IM142) from VEGF-stimulated early passage HUVEC. Conversely, bands immunoprecipitated by these antibodies from VEGF-stimulated HUVEC are cross reactive with IM142 but not an anti-flt-1 (anti-VEGFR-1) polyclonal antibody. These findings infer that the Mabs may affect the activity of KDR (VEGFR-2) in HUVEC based on experimental evidence implicating KDR (VEGFR-2) as the VEGF receptor responsible for the proliferative response in activated endothelial cells, (See example IV-3 above for protocol.)

### Example VI

### Presence of VEGF Receptor Forms on Non-Endothelial (Tumor) Cells

Several tumor lines were screened for protein reactivity with DC-101 by immunoprecipitation and detection with antiphosphotyrosine. Immunoblots from the cell lines 8161 (melanoma) and A431 (epidermoid carcinoma) yielded phosphoryiated bands with molecular weights of approximately 170 and 120 kD. These results indicate that a human VEGF receptor form is expressed in non-endothelial cells, such as tumor cells.

Similar experiments have shown that a KDR (VEGFR-2) like receptor is expressed in an ovarian carcinoma cell line, OVCAR-3. These cells also appear to secrete VEGF. Phosphoryiated bands are immunoprecipitated by an anti-KDR (VEGFR-2) polyclonal antibody from VEGF-stimulated OVCAR-3 cells that are reactive with anti-flk-1 (VEGFR-2) Mabs by western blotting. Also, bands immunoprecipitated by the murine Mabs from these cells show cross reactivity with the same polyclonal antibody. Furthermore certain murine anti-flk-1 (VEGFR-2) Mabs elicit an inhibitory effect on these cells in proliferation assays. These results demonstrate nonendothelial expression (i.e. on tumor cells) of human VEGF receptor forms. The data from the phosphorylation and proliferation assays also suggest that VEGF can modulate receptor activity in an autocrine and paracrine manner during tumorigenesis. (See Example IV-3 above for protocol.)

### Example VII

### In Vivo Studies Using DC-101

### Example VII-1

### Inhibition in Vivo of Angiogenesis by DC-101

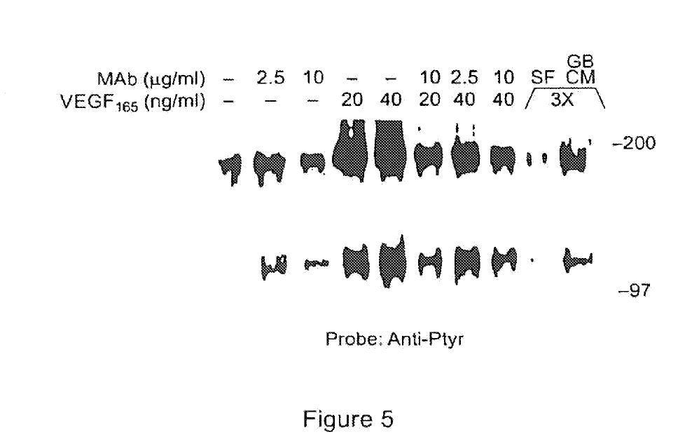

In vivo studies were designed to determine if an anti-flk-1 (VEGFR-2) monoclonal antibody would block the growth of VEGF-expressing tumor cells. In these experiments, a human glioblastoma multiform cell line was used that has high levels of VEGF message and secretes about 5 ng/ml of VEGF growth factor after a 24 hour conditioning in serum free medium (FIG. 5).

On day zero, alhymic nude mice (nu/nu; Charles River Labs) were injected in the flank with 1-2 million glioblastoma cells. Beginning on the same day, animals received intraperitoneal injections of either DC-101 and control antibodies (100 μg/animal). The mice received subsequent antibody treatments on days 3, 5, 7, 10, 12, 14, 17, 19, and 21. Animals received injections of 100 μg of either DC-101 or a control rat antibody to the murine FLK-2 (2A13) receptor on days 0, 3, 5, 7, 10, 12, 14, 17, 19, and 21 for a total inoculation of 1 mg/animal. Tumors began to appear by day 5 and followed for 50 days. Tumor size was measured daily with a caliper and tumor volume calculated by the following formula: p/6×larger diameter×(smaller diameter)² (Baselga J. Nat'l Cancer Inst. 85: 1327-1333). Measurements were taken at least three times per week and tumor volume calculated as described above. One tumor bearing animal in the DC-101 group died early in the study and was not used to determine statistical significance between the groups.

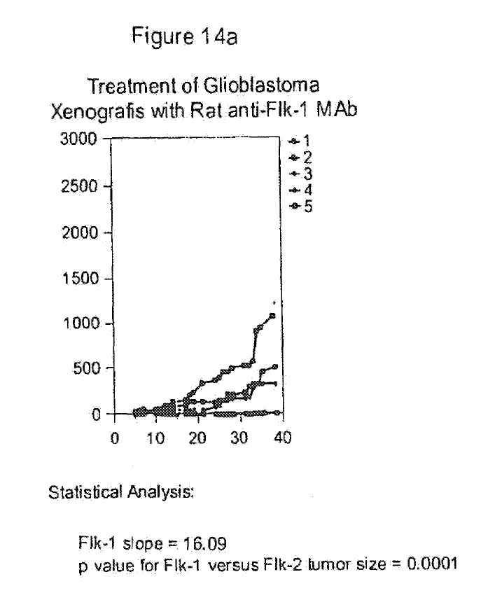

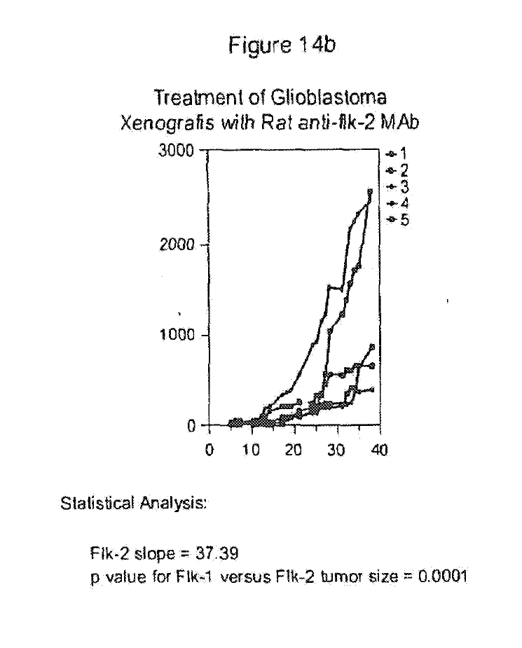

FIGS. 14a and 14b show a comparison between the DC-101 and the control 2A13 group of reduction in tumor growth over 38 days in individual animals. Although all animals developed tumors of varying sizes and number during the course of the study, DC-101-treated mice showed an overall delay in tumor progression. One mouse in the DC-101 group remained tumor free until day 49 when a small growth was observed. Even then, tumor growth was markedly suppressed. Statistical analysis of the data was done to assess differences in tumor size between the two groups. Data was subjected to a standard analysis of covariance where tumor size was regressed on time with treatment as a covariate. The results showed that reduction in tumor size over time for the DC-101 group was significantly different (p<0.0001) from that seen for 2A13 injected mice.

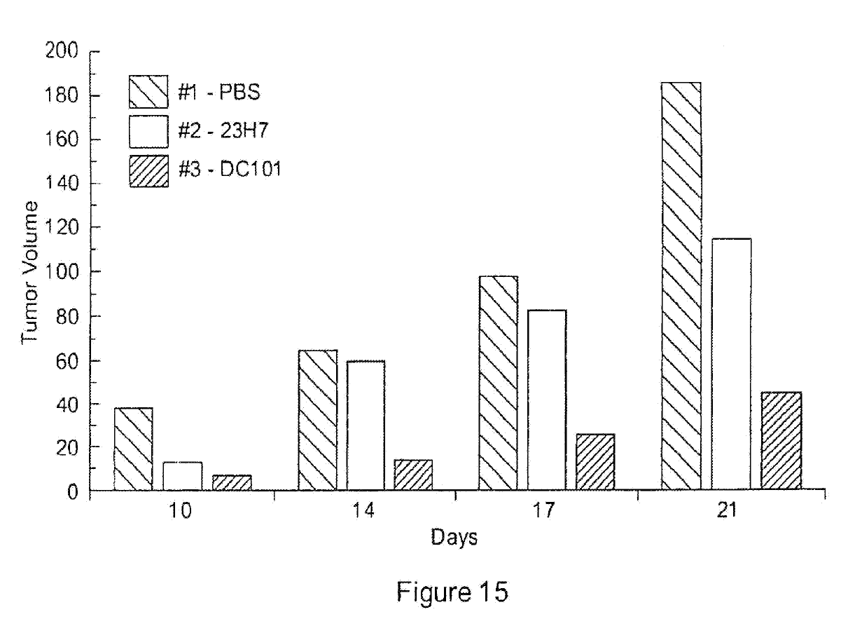

FIG. 15 shows the therapeutic efficacy of DC-101 in athymic nude mice transplanted with the human glioblastoma tumor cell line GBM-18, which secretes VEGF. Nude mice were injected subcutaneously with GBM-18 cells and divided into three groups of treatment: a PBS control, an irrelevant rat IgG1 control, and DC-101. Treatments were administered simultaneously with tumor xenografts and continued for four weeks. The results showed that GBM-18 tumor growth in DC-101-treated nude mice was significantly reduced relative to controls. This experiment indicates that DC-101 suppresses tumor growth by blocking VEGF activation of flk-1 (VEGFR-2) on tumor associated vascular endothelial cells, and that DC-101 has therapeutic value as an anti-angiogenic reagent against vascularized tumors secreting VEGF.

Monoclonal antibodies to flk-1 (VEGFR-2) receptor tyrosine kinase inhibit tumor invasion by abrogating angiogenesis. Invasive growth and angiogenesis are essential characteristics of malignant tumors. Both phenomena proved to be suitable to discriminate benign from malignant keratinocytes in a surface transplantation assay. After transplantation of a cell monolayer attached to a collagen gel onto the back muscle of nude mice, all tumor cells initially formed organized squamous epithelia, but only malignant keratinocytes grew invasively within 2-3 weeks. Both benign and malignant cells induced angiogenesis. Angiogenic response to malignant cells, however, occurred earlier, is much stronger, and capillary growth directed toward malignant epithelia. Moreover, in transplants of benign tumor cells, capillaries regressed after 2-3 weeks, whereas malignant keratinocytes maintain the level of ongoing angiogenesis. The vascular endothelial growth factor (VEGF) and its cognate receptor play a pivotal role in rumor angiogenesis. The administration of DC-101 disrupted ongoing angiogenesis leading to inhibition of tumor invasion. The antibody prevented maturation and further expansion of newly formed vascular network, but did not significantly interfere with initial angiogenesis induction. These results provide evidence that tumor invasion requires precedent angiogenesis, and that the VEGF receptors are crucial in maintaining angiogenesis in this model system.

### Example VII-2

### Effect of Different Concentrations of DC-101 on Established Glioblastoma (gbm-18) Tumors

Athymic mice (nu/nu) were inoculated subcutaneously with GBM-18 (human glioblastoma multiformae). Antibody therapy was initiated when the tumors reached an average volume of 100-200 mm³. Treatment consisted of six injections (twice weekly for 3 weeks) of the following: (i) DC-101 at 200, 400 or 800 μg/injection; (ii) an irrelevant isotype matched rat IgG (400 μg/injection); or, (iii) PBS. Tumor volumes were measured with a caliper. Tumor inhibition in the DC-101 groups was found to be significant (*) vs. the PBS and irrelevant monoclonal antibody groups.

Another experiment demonstrates the effects of the rat anti-flk-1 (VEGFR-2) monoclonal antibody DC-101 on the growth of GBM-18 tumors in nude mice. Animals (nu/nu; Charles River Labs; ten animals per group) were injected subcutaneously with GBM-18 cells (human glioblastoma [100]; 1 million per animal) on day 0. Treatments with PBS or DC-101 (200 μg per injection) were begun on day 7 and continued twice weekly for 3 weeks (6×). Graphs show a plot of the mean tumor volumes and regressed data for each group over time with their respective tumor growth rates (slopes given as λ; solid lines) and 99% confidence limits (dotted lines). The slope of the line for animals treated with DC-101 was significantly different from that of PBS (p<0.01). It is important to note that an irrelevant rat IgG1 monoclonal antibody (anti-mouse IgA; Pharmigen) had no effect on the growth of GBM-18 xenografts and gave results similar to that observed with PBS (data not shown).

### Example VIII

### Anti-flk-1 (VEGFR-2) Antibody Selectively Increases Radiation-Induced Cure Rate of Human Tumor Xenografts in Nude Mice

This example evaluates whether the monoclonal antibody DC-101 blocking the crucial VEGF receptor-2, flk-1 (VEGFR-2), on murine endothelial cells of tumor vessels increases curability of tumor xenografts by fractionated radiotherapy (RT), and whether the antibody concurrently modulates the radiation reaction of normal tissue (mouse skin).

Materials & Methods: The human small cell lung carcinoma 54A and glioblastoma multiforme U87 were implanted subcutaneously into the hind leg of nude mice. Treatment was begun when a tumor reached 5 mm in diameter (day 0). DC-101 was injected intraperitoneally every 3 days at a dose of 20 or 40 mg/kg body-weight, for a total of 6 injections. Graded total doses of radiation were given in equal daily fractions on 5 consecutive days. On day 0, a mouse received the first injection of the antibody, or RT was started. For the combined treatment, DC-101 administration was commenced on day 0, and RT was begun on day 1. Tumor size was measured 2-3 times a week after treatment. The mice with locally controlled tumors were followed-up for 90 days after the last tumor recurrence was observed in any group. Acute reaction of skin in the field of tumor irradiation was evaluated using a scoring scale during the first 30 days after the beginning of RT.

Results: The antibody used alone induced growth inhibition (but not regression) of both tumors in a dose-dependent manner. The effect was more pronounced in 54A than in U87 xenografts. In combination with the lowest doses of radiation (25-30 Gy total), DC-101 provided an additional tumor growth delay when compared with RT alone, in either model. The antibody, also in a dose-dependent fashion, augmented the curative effect of RT. For example, at its higher dose, DC-101 decreased the dose of radiation necessary to control 50% of tumors locally: 1.7 fold in 54A xenografts (from 67.6 Gy for RT alone to 39.1 Gy for the combined therapy), and 1.3 fold in U87 (from 97.8 to 74.8 Gy). It is also of particular importance that such effects of DC-101 were selective for tumors. That is, no parallel changes of skin radiation reaction by the antibody were detected. As assessed in additional experiments, the DC-101-induced enhancement of the radiation response of tumors was not associated with their radiosensitization or changes in oxygenation, while correlated with a significant decrease of the tumor interstitial fluid pressure by the antibody.

Conclusion: The results collectively suggest that the blockage of VEGF-signaling pathways by an antibody against the main receptor to these growth factor molecules can selectively potentiate the tumor curative response to fractionated RT; and thus, provide a therapeutic gain.

### Example IX

### Producing Single Chain Antibodies

### Example IX-1 Materials

### Example IX-1 (a)

### Cell Lines and Proteins

Primary-cultured HUVEC was maintained in EBM-2 medium at 37° C., 5% CO2. Cells were used between passage 2-5 for all assays. VEGF₁₆₅ protein was expressed in baculovirus and purified. Complementary DNA encoding the extracellular domain of KDR (VEGFR-2) was isolated by RT-PCR from human fetal kidney mRNA and subcloned into the Bgl II and BspE I sites of the vector AP-Tag. In this plasmid the cDNA for KDR (VEGFR-2) extracellular domain is fused in-frame with the cDNA for human placental AP. The plasmid was electroporated into NIH 3T3 cells together with the neomycin expression vector pSV-Neo and stable cell clones were selected with G418. The soluble fusion protein KDR-AP was purified from cell culture sup era at ant by affinity chromatography using immobilized monoclonal antibodies to AP.

### Example IX-1 (b)

### Mice Immunization and Construction of Single Chain Antibody Phage Display Library

Female BALB/C mice were given two intraperitoneal (i.p.) injections of 10 μg KDR-AP in 200 ul of RIBI Adjuvant System followed by one i.p. injection without RIBI adjuvant over a period of two months. The mice were also given a subcutaneous (s.c.) injection of 10 μg KDR-AP in 200 μl of RIBI at the time of the first immunization. The mice were boosted i.p. with 20 μg of KDR-AP three days before euthanasia. Spleens from donor mice were removed and the cells were isolated. RNA was extracted and mRNA was purified from total RNA of splenocytes. A scFv phage display library was constructed using the mRNA which was displayed on the surface of the filamentous phage M13.

In displaying the scFv on filamentous phage surface, antibody V_{H} and V_{L} domains are joined together by a 15 amino-acid-long linker (GGGGS)³ and fused to the N-terminal of phage protein III. A 15 amino-acid-long E tag, which is followed by an amber codon (TAG), was inserted between the C-terminal of V_{L} and the protein III for detection and other analytic purposes. The amber codon positioned between the E tag and the protein III enables the construct to make scFv in surface-displaying format when transformed into a suppressor host (such as TG1 cells) and scFv in soluble form when transformed into a nonsupressor host (such as HB2151 cells).

The assembled scFv DNA was ligated into the pCANTAB 5E vector. The transformed TG1 cells were plated onto 2YTAG plates and incubated. The colonies were scraped into 10 ml of 2YT medium, mixed with 5 ml 50% glycerol and stored at −70° C. as the library stock.

### Example IX-1(c)

### Biopanning

The library stock was grown to log phase, rescued with M13K07 helper phage and amplified overnight in 2YTAK medium (2YT containing 100 μg/ml of ampicillin and 50 μg/ml of kanamycin) at 30° C. The phage preparation was precipitated in 4% PEG/0.5M NaCl, resuspended in 3% fat-free milk/PBS containing 500 μg/ml of AP protein and incubated at 37° C. for 1 h to capture phage displaying anti-AP scFv and to block other nonspecific binding.

KDR-AP (10 μg/ml) coated Maxisorp Star tubes (Nunc, Denmark) were first blocked with 3% milk/PBS at 37° C. for 1 h, and then incubated with the phage preparation at room temperature for 1 h. The tubes were washed 10 times with PBST followed by 10 times with PBS (PBS containing 0.1% Tween 20). The bound phage was eluted at room temperature for 10 min. with 1 ml of a freshly prepared solution of 100 mM triethylamine. The eluted phage were incubated with 10 ml of mid-log phase TG1 cells at 37° C. for 30 min. stationary and 30 min. shaking. The infected TG1 cells were then plated onto 2YTAG plates and incubated overnight at 30° C.

Ninety-nine percent (185/186) of clones screened after the third round of panning were found to be specific KDR (VEGFR-2) binders. However, only 15 (8%) of these binders could block KDR (VEGFR-2) binding to immobilized VEGF. DNA BstN I fingerprinting of these 15 clones indicated the presence of 2 different digestion patterns; whereas 21 randomly picked VEGF nonblockers yielded 4 different patterns. All the digestion patterns were also seen in clones identified after the second round of panning. Representative clones of each digestion pattern were picked from clones recovered after the 2nd round of panning and subject to DNA sequencing. Out of 15 clones sequenced, 2 unique VEGF blockers and 3 nonblockers were identified. One scFv, p2A7, which neither binds to KDR (VEGFR-2) nor blocks VEGF binding to KDR (VEGFR-2), was selected as a negative control for all studies.

### Example IX-1 (d)

### Phage ELISA

Individual TG1 clones were grown at 37° C. in 96 well plates and rescued with M13K07 helper phage as described above. The amplified phage preparation was blocked with ⅙ volume of 18% milk/PBS at RT for 1 h and added to Maxi-sorp 96-well microliter plates (Nunc) coated with KDR-AP or AP (1 μg/ml×100 ul). After incubation at room temperature for 1 h, the plates were washed 3 times with PBST and incubated with a rabbit anti-M13 phage Ab-HRP conjugate. The plates were washed 5 times, TMB peroxidase substrate added, and the OD at 450 nm read using a microplate reader and scFv antibodies were identified and sequenced.

### Example IX-1(e)

### Preparation of Soluble scFv

Phage of individual clones were used to infect a nonsuppressor E.coli host HB2151 and the infectant selected on 2YTAG-N plates. Expression of scFv in HB2 151 cells was induced by culturing the cells in 2YTA medium containing 1 mM isopropyl-1-thio-B-D-galactopyranoside at 30° C. A periplasmic extract of the cells was prepared by resuspending the cell pellet in 25 mM Tris (pH 7.5) containing 20% (w/v) sucrose, 200 mM NaCl, 1 mM EDTA and 0.1 mM PMSF, followed by incubation at 4° C. with gentle shaking for 1 h. After centrifugation at 15,000 rpm for 15 min., the soluble scFv was purified from the supernatant by affinity chromatography using the RPAS Purification Module (Pharmacia Biotech).

### Example IX-2

### Assays

### Example IX-2 (a)

### Quantitative KDR (VEGFR-2) Binding Assay

Two assays were employed to examine quantitatively the binding of purified soluble scFv to KDR (VEGFR-2).

Four different clones, including the two VEGF blockers, p1C11 and p1F12, one nonblocker, the dominant clone p2A6 and the nonbinder p2A7, were expressed in shaker flasks using a nonsuppressor host E.coli HB2151 cells. The soluble scFv was purified from the periplasmic extracts of E.coli by anti-E-tag affinity chromatography. The yield of purified scFv of these clones ranged from 100-400 μg/liter culture.

In the direct binding assay, various amounts of soluble scFv were added to KDR (VEGFR-2)-coated 96-well Maxi-sorp microtiter plates and incubated at room temperature for 1 h, after which the plates were washed 3 times with PBST. The plates were then incubated at room temperature for 1 h with 100 μl of mouse anti-E tag antibody followed by incubation with 100 μl of rabbit anti-mouse antibody-HRP conjugate. The plates were washed and developed following the procedure described above for the phage ELISA.

In another assay, i.e., the competitive VEGF blocking assay, various amounts of soluble scFv were mixed with a fixed amount of KDR-AP (50 ng) and incubated at room temperature for 1 h. The mixture were then transferred to 96-well microtiter plates coated with VEGF₁₆₅ (200 ng/well) and incubated at room temperature for an additional 2 h, after which the plates were washed 5 times and the substrate for AP was added to quantify the bound KDR-AP molecules. IC₅₀, i.e., the scFv concentration required for 50% inhibition of KDR (VEGFR-2) binding to VEGF, was then calculated.

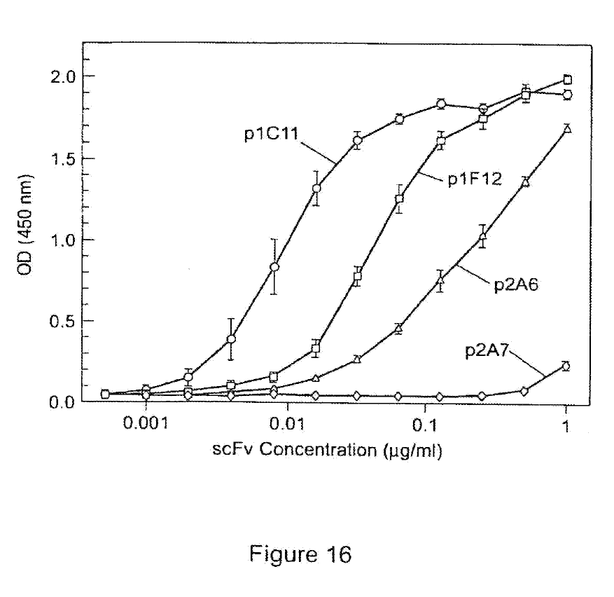

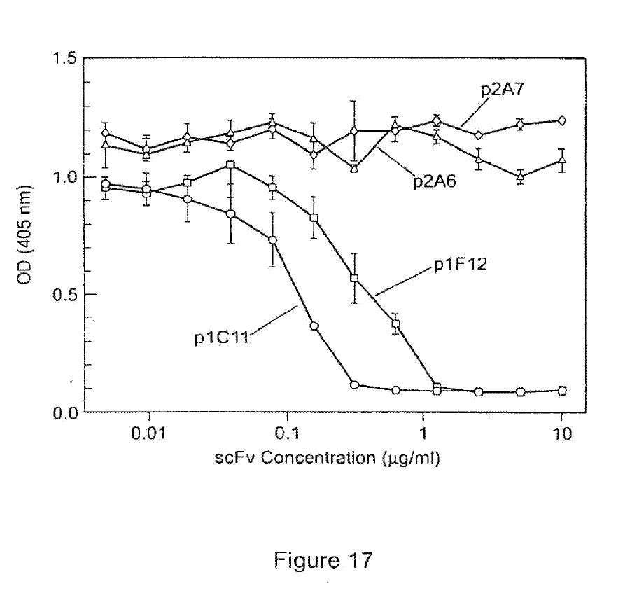

FIG. 16 shows the dose-dependent binding of scFv to immobilized KDR (VEGFR-2) as assayed by a direct binding ELISA. Clone p1C11 and p1F12, but not p2A6, also block KDR (VEGFR-2) binding to immobilized VEGF as shown in FIG. 17. Data shown in FIG. 17 are the means±SD of triplicate determinations. The negative control clone, P2A7, did not bind to KDR (VEGFR-2) nor block KDR (VEGFR-2) binding to VEGF (FIGS. 16 and 17). Clone p1C11, the dominant clone after each round of panning, showed the highest KDR (VEGFR-2) binding capacity and the highest potency in blocking VEGF binding to KDR (VEGFR-2) (Table 1). The antibody concentrations of clone p1C11 required for 50% of maximum binding to KDR (VEGFR-2) and for 50% of inhibition of KDR (VEGFR-2) binding to VEGF (FIG. 17) were 0.3 nM and 3 nM, respectively (See Table 1). FACS analysis demonstrated that p1C11, p1F12 and p2A6 were also able to bind to cell surface expressed receptor on HUVEC.

### Example IX-2 (b)

### BIAcore Analysis of the Soluble scFv

The binding kinetics of soluble scFv to KDR (VEGFR-2) were measured using BIAcore biosensor (Pharmacia Biosensor). KDR-AP fusion protein was immobilized onto a sensor chip and soluble scFv were injected at concentrations ranging from 62.5 nM to 1000 nM. Sensorgrams were obtained at each concentration and were evaluated using a program, BIA Evaluation 2.0, to determine the rate constant kon and koff. Kd was calculated from the ratio of rate constants koff/kon.

Table 1 shows the results of the surface plasmon resonance on a BIAcore instrument. The VEGF-blocking scFv, p1C11 and p1F12, bound to immobilized KDR (VEGFR-2) with Kd of 2.1 and 5.9 nM, respectively. The non-blocking scFv, p2A6, bound to KDR (VEGFR-2) with approximately a 6-fold weaker affinity (Kd, 11.2 nM) than the best binder p1C11, mainly due to a much faster dissociation rate. As anticipated, p2A7 did not bind to the immobilized KDR (VEGFR-2) on the BIAcore.

### Example IX-2 (c)

### Phosphorylation Assay

Phosphorylation assays were performed with early passage HUVEC following a protocol described previously. Briefly, HUVEC were incubated in serum free EBM-2 base medium supplemented with 0.5% bovine serum albumin at room temperature for 10 min. in the presence or absence of scFv antibodies at 5 μg/ml, followed by stimulation with 20 ng/ml VEGF₁₆₅ at room temperature for an additional 15 min. The cells were lysed and the KDR (VEGFR-2) receptor was immunoprecipitated from the cell lysates with Protein A Sepharose beads coupled to a rabbit anti-KDR (anti-VEGFR-2) polyclonal antibody (ImClone Systems Incorporated). The beads were washed, mixed with SDS loading buffer, and the supernatant subjected to Western blot analysis. To detect KDR (VEGFR-2) phosphorylation, blots were probed with an anti-phosphotyrosine Mab, 4G10. For the MAP kinase activity assay, cell lysates were resolved with SDS-PAGE followed by Western blot analysis using a phospho-specific MAP kinase antibody. All signals were detected using ECL.

Results showed that VEGF-blocking scFv p1C11, but not the non-blocking scFv p2A6, was able to inhibit KDR (VEGFR-2) receptor phosphorylation stimulated by VEGF. Further, p1C11 also effectively inhibited VEGF-stimulated activation of MAP kinases p44/p42. In contrast, neither p1C11, nor p2A6 inhibited FGF-stimulated activation of MAP kinases p44/p42.

### Example IX-2 (d)

### Anti-Mitogenic Assay

HUVEC (5×103 cells/well) were plated onto 96-well tissue culture plates (Wallach, Inc., Gaithersburg, Md.) in 200 ul of EBM-2 medium without VEGF, bFGF or EGF and incubated at 37° C. for 72 h. Various amounts of antibodies were added to duplicate wells and pre-incubated at 37° C. for 1 h, after which VEGFRs was added to a final concentration of 16 ng/ml. After 18 h of incubation, 0.25 μCi of [3H]-TdR (Amersham) was added to each well and incubated for an additional 4 h. The cells were placed on ice, washed twice with serum-containing medium, followed by a 10 minute incubation at 4° C. with 10% TCA. The cells were then washed once with water and solubilized in 25 μl of 2% SDS. Scintillation fluid (150 μl/well) was added and DNA incorporated radioactivity was determined on a scintillation counter (Wallach, Model 1450 Microbeta Scintillation Counter).

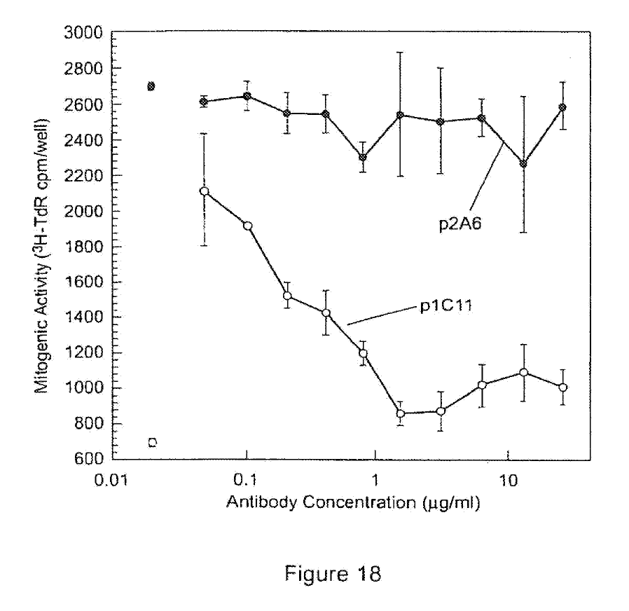

The ability of scFv antibodies to block VEGF-stimulated mitogenic activity on HUVEC is shown in FIG. 18. The VEGF-blocking scFv p1C11 strongly inhibited VEGF induced DNA synthesis in HUVEC with an EC₅₀, i.e., the antibody concentration that inhibited 50% of VEGF-stimulated mitogenesis of HUVEC, of approximately 5 nM. The non-blocking scFv p2A6 showed no inhibitory effect on the mitogenic activity of VEGF. Neither p1C11 nor p2A6 inhibited bFGF-induced DNA synthesis in HUVEC (not shown). Data shown in FIG. 18 are representative of at least three separate experiments. (!) VEGF only; (∀) no VEGF.

### Example IX-3

### Producing Chimeric Antibodies from p1C11

### Example IX-3(a)

### Cell Lines and Proteins

Primary-cultured human umbilical vein endothelial cells (HUVEC) were maintained in EBM-2 medium at 37° C., 5% CO2. Cells between passage 2-5 were used for all assays. VEGF₁₆₅ and KDR (VEGFR-2)-alkaline phosphatase fusion proteins (KDR-AP) were expressed in baculovirus and NIH 3T3 cells, respectively, and purified following the procedures described above. The anti-KDR (anti-VEGFR-2) scFv p1C11 and scFv p2A6, an antibody that binds to KDR (VEGFR-2) but does not block KDR (VEGFR-2)-VEGF interaction, were isolated from a phage display library constructed from a mouse immunized with KDR (VEGFR-2) as described above. C225 is a chimeric IgG1 antibody directed against epidermal growth factor (EGF) receptor. See above.

### Example IX-3(b)

### Cloning of the Variable Domains of scFv p1C11

The variable domains of the light (V_{L}) (SEQ ID NO: 8 and SEQ ID NO: 16) and the heavy (V_{H}) (SEQ ID NO: 7 and SEQ ID NO: 15) chains of p1C11 were cloned from the scFv expression vector by PCR using primers 1 and 2, and primers 3 and 4, respectively. The leader peptide sequence for protein secretion in mammalian cells was then added to the 5′ of the V_{L} and the V_{H} by PCR using primers 5 and 2, and primers 5 and 4, respectively.

Primer 1:

[SEQ ID No:37]

5′ CTA GTA GCA ACT GCA ACT GGA GTA CAT TCA GAC ATC
GAG CTC3′
Primer 2:

[SEQ ID NO:38]

5′ TCG ATC TAG AAG GAT CCA CTC ACG TTT TAT TTC
CAG3′BamHI
Primer 3:

[SEQ ID No:39]

5′ CTA GTA GCA ACT GCA ACT GGA GTA CAT TCA CAG GTC
AAG CTG3′
Primer 4:

[SEQ ID No:40]

5′ TCG AAG GAT CCA CTC ACC TGA GGA GAC GGT3′BamHI
Primer 5:

[SEQ ID No:41]

5′ GGT CAA AAG CTT ATG GGA TGG TCA TGT ATC ATC CTT
TTT Hind III CTA GTA GCA ACT3′

### Example IX-3(c)

### Construction of the Expression Vectors for the Chimeric p1C11 IgG

Separate vectors for expression of chimeric IgG light chain and heavy chains were constructed. The cloned V_{L} gene was digested with Hind III and BamH I and ligated into the vector pKN100 containing the human κ light chain constant region (C_{L}) to create the expression vector for the chimeric p1C11 light chain, c-p1C11-L. The cloned V_{H} gene was digested with Hind III and BamH I and ligated into the vector pGID105 containing the human IgG1 (γ) heavy chain constant domain (C_{H}) to create the expression vector for the chimeric p1C11 heavy chain, c-p1C11-H. Both constructs were examined by restriction enzyme digestion and verified by dideoxynucleolide sequencing.

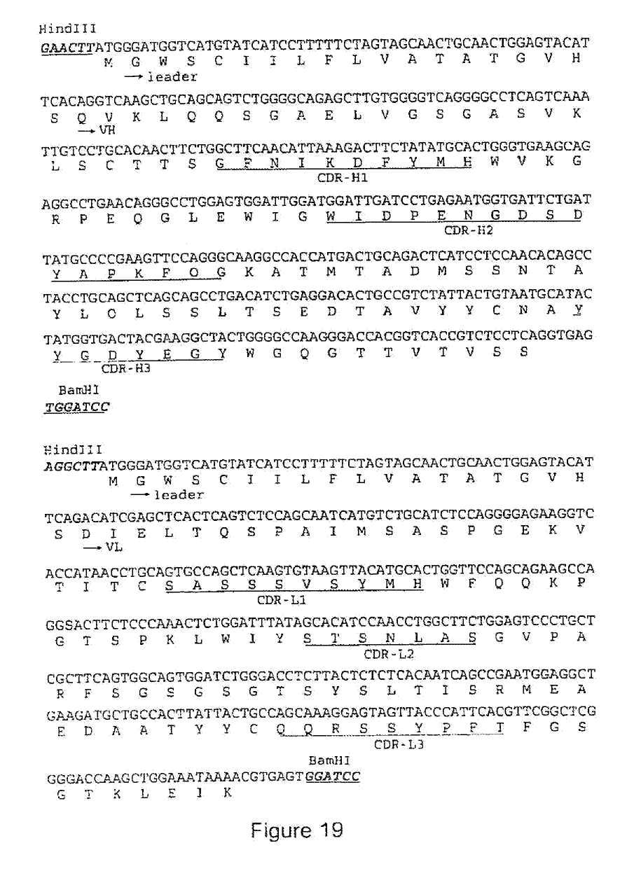

As seen in FIG. 19 both the V_{H} and the V_{L} domains are precisely fused on their 5′ ends to a gene segment encoding a leader peptide sequence (SEQ ID NO: 23 and SEQ ID NO: 24) as marked. The V_{H} and the V_{L} domains are ligated via Hind III/BamH I sites into expression vector pG1D105, which contains a cDNA version of the human γ1 constant region gene, and pKN100, which contains a cDNA version of the human κ chain constant region gene, respectively. In each case, expression is under control of the HCMVi promoter and terminated by an artificial termination sequence. The light and the heavy chain complimentarily determining region (CDR) residues, defined according the hypervariable sequence definition of Kabat et al., are underlined and labeled CDR-H1 to H3 and CDR-L1 to L3, respectively. CDR-H1 (SEQ ID NO: 1 and SEQ ID NO: 9); CDR-H2 (SEQ ID NO: 2 and SEQ ID NO: 10); CDR-H3 (SEQ ID NO: 3 and SEQ ID NO: 11); CDR-L1 (SEQ ID NO: 4 and SEQ ID NO: 12); CDR-L2 (SEQ ID NO: 5 and SEQ ID NO: 13); CDR-L3 (SEQ ID NO: 6 and SEQ ID NO: 14).

### Example IX-3(d)

### IgG Expression and Purification

COS cells were co-transfected with equal amounts of c-p1C11-L and c-p1C11-H plasmids for transient IgG expression. Subconfluent COS cells grown in DMEM/10% FCS in 150 mm culture dishes were rinsed once with 20 ml of DMEM containing 40 mM Tris (pH 7.4), followed by incubation at 37° C. for 4.5 h with 4 ml of DMEM/DEAE-Dextran/DNA mixture (DMEM containing 40 mM Tris, 0.4 mg/ml of DEAE-Dextran (Sigma), and 20 μg each of c-p1C11-L and c-p1C11-H plasmids). The cells were incubated at 37° C. for 1 h with 4 ml of DMEM/2% FCS containing 100 nM of chloroquine (Sigma), followed by incubation with 1.5 ml of 20% glycerol/PBS at room temperature for 1 min. The cells were washed twice with DMEM/5% FCS and incubated in 20 ml of the same medium at 37° C. overnight. The cell culture medium was changed to serum-free DMEM/HEPES after the cells were washed twice with plain DMEM. The cell culture supernatant was collected at 48 h and 120 h after the transfection. The chimeric IgG was purified from the pooled supernatant by affinity chromatography using Protein G column following the protocol described by the manufacturer (Pharmacia Biotech). The IgG-containing fractions were pooled, buffer exchanged into PBS and concentrated using Centricon 10 concentrators (Amicon Corp., Beverly, Mass.). The purity of the IgG was analyzed by SDS-PAGE. The concentration of purified antibody was determined by ELISA using goat anti-human y chain specific antibody as the capture agent and HRP-conjugated goat anti-human k chain antibody as the detection agent. Standard curve was calibrated using a clinical grade antibody, C225.

After affinity purification by Protein G, a single protein band of ˜150 kD was seen in SDS-PAGE. Western blot analysis using HRP-conjugated anti-human IgG1 Fc specific antibody confirmed the presence of human IgG Fc portion in the purified protein (not shown).

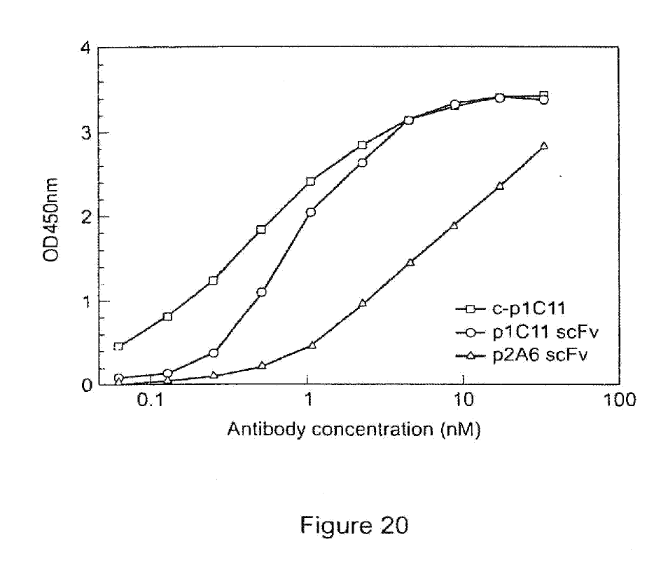

The results of the ELISA show that c-p1C11 binds more efficiently to immobilized KDR (VEGFR-2) than the parent scFv (FIG. 20).

### Example IX-4

### Assays and Analysis

### Example IX-4(a)

### FACS Analysis

Early passage HUVEC cells were grown in growth factor-depleted EBM-2 medium overnight to induce the expression of KDR (VEGFR-2). The cells were harvested and washed three times with PBS, incubated with c-p1C11 IgG (5 μg/ml) for 1 h at 4° C., followed by incubation with a FITC labeled rabbit anti-human Fc antibody (Capper, Organon Teknika Corp., West Chester, Pa.) for an additional 60 min. The cells were washed and analyzed by a flow cytometer (Model EPICS, Coulter Corp., Edison, N.J.).

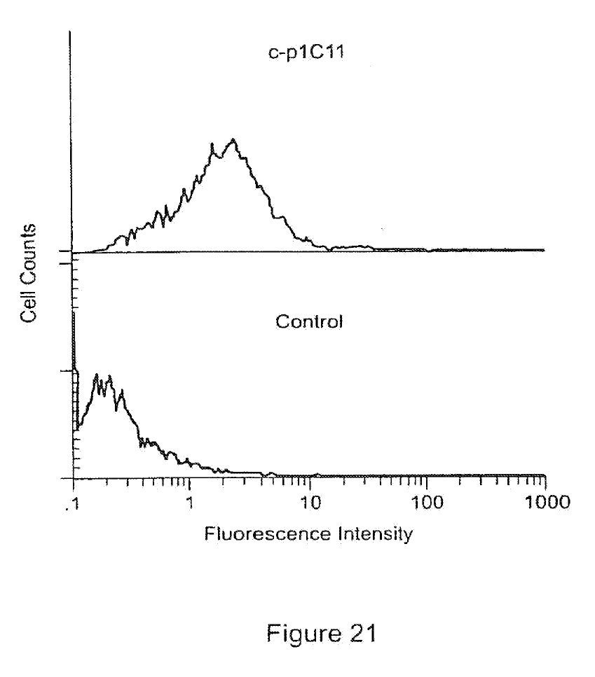

FIG. 21 is a graph showing the FACS analysis of c-p1C11 binding to KDR (VEGFR-2)-expressing HUVEC. As previously seen with the parent scFv p1C11, c-p1C11 binds specifically to KDR (VEGFR-2) expressed on early passage HUVEC.

### Example IX-4(b)

### Quantitative KDR (VEGFR-2) Binding Assay

Various amounts of antibodies were added to KDR (VEGFR-2)-coated 96-well Maxi-sorp microtiter plates (Nunc. Danmark) and incubated at room temperature for 1 h, after which the plates were washed 3 times with PBS containing 0.1% Tween-20. The plates were then incubated at RT for 1 h with 100 ul of mouse anti-E tag antibody-HRP conjugate (Pharmacia Biotech) for the scFv, or rabbit anti-human IgG Fc specific antibody-HRP conjugate (Cappel, Organon Teknika Corp.) for the chimeric IgG. The plates were washed 5 times, TMB peroxidase substrate (KPL, Gaithersburg, Md.) added, and the OD at 450 nm read using a microplate reader (Molecular Device, Sunnyvale, Calif.).

FIG. 20 is a graph showing the direct binding of antibodies to immobilized KDR (VEGFR-2). C-p1C11 is shown to bind more efficiently to immobilized KDR (VEGFR-2) receptor than the parent scFv.

### Example IX-4(c)

### BIA Core Analysis

The binding kinetics of antibodies to KDR (VEGFR-2) were measured using BIAcore biosensor (Pharmacia Biosensor). KDR (VEGFR-2)-AP fusion protein was immobilized onto a sensor chip, and antibodies or VEGF were injected at concentrations ranging from 25 nM to 200 nM. Sensorgrams were obtained al each concentration and were evaluated using a program, BIA Evaluation 2.0, to determine the rate constants kon and koff. Kd was calculated as the ratio of rate constants koff/kon.

BIAcore analysis reveals that c-p1C11 bind to KDR (VEGFR-2) with higher affinity than the parent scFv (Table 2). The Kd of c-p1C11 is 0.82 nM, compared to 2.1 nM for the scFv. The increased affinity of c-p1C11 is mainly due to a slower dissociation rate (koff) of the bivalent chimeric IgG. It is important to note that the affinity (Kd) of c-p1C11 for binding to KDR (VEGFR-2) is similar to that of the natural ligand VEGF for binding to KDR (VEGFR-2), which is 0.93 nM as determined in our BIAcore analysis (Table 2).

### Example IX-4(d)

### Competitive VEGF Binding Assay

In the first assay, various amounts of antibodies were mixed with a fixed amount of KDR (VEGFR-2)-AP (50 ng) and incubated at room temperature for 1 h. The mixtures were then transferred to 96-well microtiter plates coated with VEGF₁₆₅ (200 ng/well) and incubated at room temperature for an additional 2 h, after which the plates were washed 5 times and the substrate for AP (p-nitrophenyl phosphate, Sigma) was added to quantify the bound KDR (VEGFR-2)-AP molecules. EC₅₀, i.e., the antibody concentration required for 50% inhibition of KDR (VEGFR-2) binding to VEGF, was then calculated.

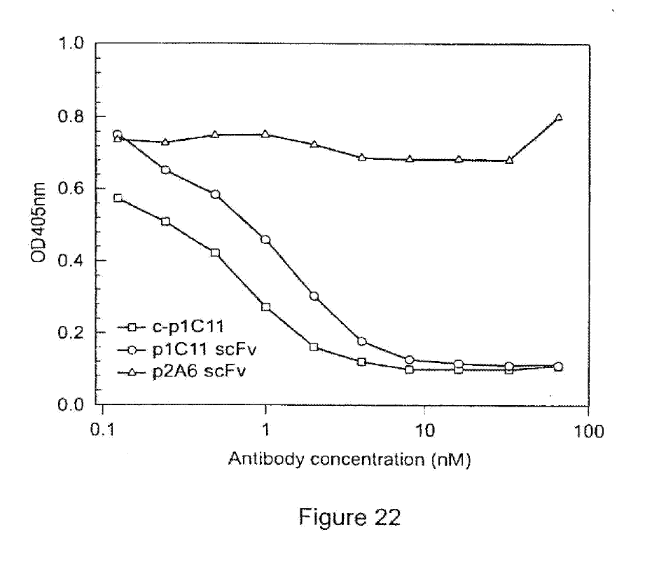

FIG. 22 shows that c-p1C11 block KDR (VEGFR-2) receptor from binding to immobilized VEGF in a dose-dependent manner. The chimeric antibody is more potent in blocking VEGF-KDR (VEGFR-2) interaction with an IC₅₀ (i.e., the antibody concentrations required to inhibit 50% of KDR (VEGFR-2) from binding to VEGF) of 0.8 nM, compared to that of 2.0 nM for the scFv. The control scFv p2A6 also binds KDR (VEGFR-2) (FIG. 20) but does not block VEGF-KDR (VEGFR-2) interaction (FIG. 22).

In the second assay, various amounts of c-p1C11 antibody or cold VEGF₁₆₅ protein were mixed with a fixed amount of 125I labeled VEGF₁₆₅ and added to 96-well microtiter plates coated with KDR (VEGFR-2) receptor. The plates were incubated at room temperature for 2 h, washed 5 times and the amounts of radiolabeled VEGF₁₆₅ that bound to immobilized KDR (VEGFR-2) receptor were counted. Concentrations of c-p1C11 and cold VEGF₁₆₅ required to block 50% of binding of the radiolabeled VEGF to immobilized KDR (VEGFR-2) receptor were determined.

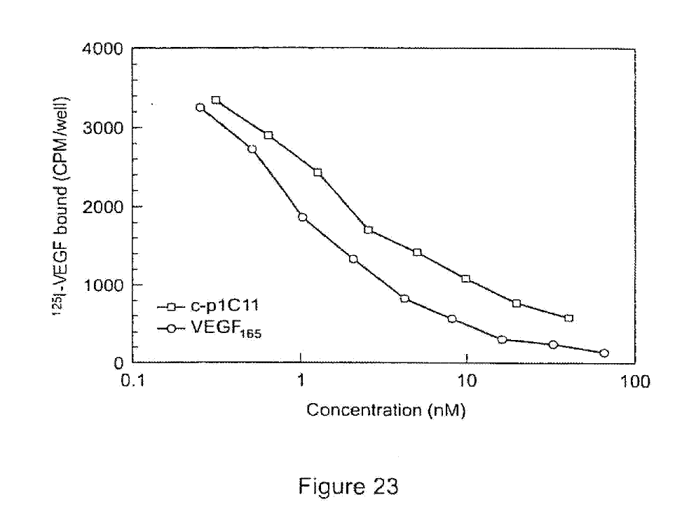

The results of the inhibition of binding of radiolabeled VEGF₁₆₅ is shown in FIG. 23. The data shown are the means of triplicate determinations. c-p1C11 is shown to efficiently compete with ¹²⁵I labeled VEGF for binding to immobilized KDR (VEGFR-2) receptor in a dose-dependent manner. As expected, C225, a chimeric antibody directed against EGF receptor does not bind to KDR (VEGFR-2) receptor or block VEGF-KDR (VEGFR-2) interaction (not shown).

### Example IX-4(e)

### Phosphorylation Assay

Subconfluent HUVEC cells were grown in growth factor depleted EBM-2 medium for 24 to 48 h prior to experimentation. After pretreatment with 50 nM sodium orthovanadate for 30 min, the cells were incubated in the presence or absence of antibodies for 15 min, followed by stimulation with 20 ng/ml of VEGF₁₆₅, or 10 ng/ml of FGF at room temperature for an additional 15 min. The cells were then lysed in lysis buffer (50 nM Tris, 150 mM NaCl, 1% NP-40, 2 mM EDTA, 0.25% sodium deoxycholate, 1 mM PMSF, 1 μg/ml leupeptin, 1 μg/ml pepstatin, 10 μg/ml aprotinin, pH 7.5) and the cell lysate used for both the KDR (VEGFR-2) and MAP kinase phosphorylation assays. The KDR (VEGFR-2) receptor was immunoprecipitated from the cell lysates with Protein A Sepharose beads (Santa Cruz Biotechnology, Inc., CA) coupled to an anti-KDR (VEGFR-2) antibody, Mab 4.13 (ImClone Systems). Proteins were resolved with SDS-PAGE and subjected to Western blot analysis. To detect KDR (VEGFR-2) phosphorylation, blots were probed with an antipliosphotyrosine Mab, PY20 (ICN Biomedicals, Inc. Aurora, Ohio). For the MAP kinase activity assay, cell lysates were resolved with SDS-PAGE followed by Western blot analysis using a phospho-specific MAP kinase antibody (New England BioLabs, Beverly, Mass.). All signals were detected using ECL (Amersham, Arlington Heights, Ill.). In both assays, the blots were reprobed with a polyclonal anti-KDR (VEGFR-2) antibody (ImClone Systems) to assure that equal amount of protein was loaded in each lane of SDS-PAGE gels.

C-p1C11 effectively inhibits VEGF-stimulated phosphorylation of KDR (VEGFR-2) receptor and activation of p44/p42 MAP kineses. In contrast, C225 does not show any inhibition of VEGF-stimulated activation of KDR (VEGFR-2) receptor and MAP kineses. Neither c-p1C11, nor C225 alone has any effects on the activity of KDR (VEGFR-2) receptor and p44/p42 MAP kinases. As previously seen with the scFv p1C11, c-p1C11 does not inhibit FGF-stimulated activation of p44/p42 MAP kinases (not shown). Furthermore, neither scFv p2A6, nor the chimeric IgG form of p2A6 (c-p2A6), inhibits VEGF-stimulated activation of KDR (VEGFR-2) receptor and MAP kineses (not shown).

### Example IX-4(f)

### Anti-Mitogenic Assay

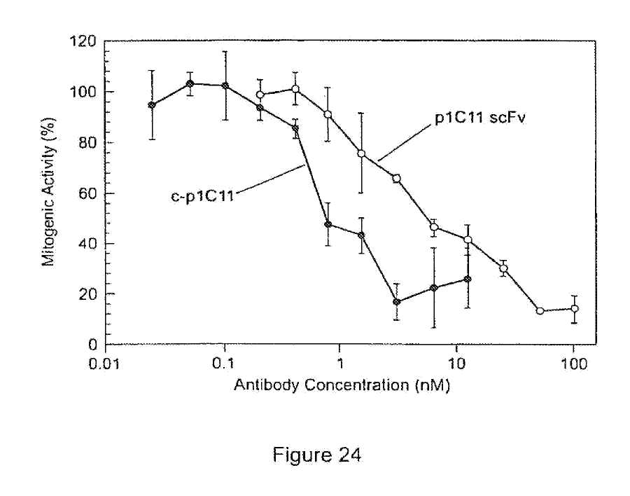

The effect of anti-KDR (VEGFR-2) antibodies on VEGF-stimulated mitogenesis of human endothelial cells was determined with a [³H]-TdR DNA incorporation assay using HUVEC. HUVEC (5×10³ cells/well) were plated into 96-well tissue culture plates in 200 μl of EBM-2 medium without VEGF, bFGF or EGF and incubated at 37° C. for 72 h. Various amounts of antibodies were added to duplicate wells and pre-incubated at 37° C. for 1 hour, after which VEGF₁₆₅ was added to a final concentration of 16 ng/ml. After 18 hours of incubation, 0.25 μCi of [3H]-TdR was added to each well and incubated for an additional 4 hours. DNA incorporated radioactivity was determined with a scintillation counter. The data shown in FIG. 24 are representative of at least three separate experiments.

Both c-p1C11 and scFv p1C11 effectively inhibit mitogenesis of HUVEC stimulated by VEGF (FIG. 24). C-p1C11 is a stronger inhibitor of VEGF-induced mitogenesis of HUVEC than the parent scFv. The antibody concentrations required to inhibit 50% of VEGF-induced mitogenesis of HUVEC are 0.8 nM for c-p1C11 and 6 nM for the scFv, respectively. As expected, scFv p2A6 does not show any inhibitory effect on VEGF-stimulated endothelial cell proliferation.

The invention as claimed is enabled in accordance with the above specification and readily available references and starting materials. Nevertheless, Applicants have deposited with the American Type Culture Collection, 12301 Parklawn Drive, Rockville, Md., 20852 USA (ATCC) the hybridoma cell lines that produce the monoclonal antibodies listed below:

Hybridoma cell line DC-101 producing rat anti-mouse flk-1 (VEGFR-2) monoclonal antibody deposited on Jan. 26, 1994 (ATCC Accession Number HB 11534).

Hybridoma cell line M25.18A1 producing mouse anti-mouse flk-1 (VEGFR-2) monoclonal antibody Mab 25 deposited on Jul. 19, 1996 (ATCC Accession Number HB 12152).

Hybridoma cell line M73.24 producing mouse anti-mouse flk-1 (VEGFR-2) monoclonal antibody Mab 73 deposited on Jul. 19, 1996 (ATCC Accession Number HB 12153).

These deposits were made under the provisions of the Budapest Treaty on the International Recognition of the Deposit of Microorganisms for the Purposes of Patent Procedure and the regulations thereunder (Budapest Treaty). This assures maintenance of a viable culture for 30 years from date of deposit. The organism will be made available by ATCC under the terms of the Budapest Treaty, and subject to an agreement between Applicants and ATCC, which assures unrestricted availability upon issuance of the pertinent U.S. patent. Availability of the deposited strains is not to be construed as a license to practice the invention in contravention of the rights granted under the authority of any government in accordance with its patent laws.

TABLE 1
KDR (VEGFR-2) - BINDING ANALYSIS OF ANTI-KDR
(VEGFR-2) ScFv ANTIBODIES

| | KDR | VEGF | Binding Kinetics³ | | |
|---|---|---|---|---|---|
| ScFv | Binding¹ | Binding² | kon | koff | Kd |
| Clone | (ED₅₀ nM) | (IC₅₀ nM) | (10⁵ M⁻¹s⁻¹) | (10⁻⁴s⁻¹) | (10⁻⁹ M) |
| P1C11 | Yes (0.3) | Yes (3.0) | 1.1 | 2.3 | 2.1 |
| P1F12 | Yes (1.0) | Yes (15) | 0.24 | 1.4 | 5.9 |
| P2A6 | Yes (5.0) | No (>300) | 4.1 | 46.1 | 11.2 |
| P2A7 | No (N/A) | No (>300) | N/A | N/A | N/A |

¹Determined by direct binding ELISA, numbers in the parenthesis represent the scFv concentrations that give 50% of maximum binding;
²Determined by competitive VEGF blocking ELISA, numbers in the parenthesis represent the scFv concentrations required for 50% inhibition of KDR binding to immobilized VEGF;
³Determined by BIAcore analysis. NA = not applicable.

TABLE 2
BINDING KINETICS OF P1C11 SCFV AND C-P1C11 TO
KDR (VEGFR-2) RECEPTOR.*

| | kon | koff | Kd |
|---|---|---|---|
| Antibody | (10⁵ M⁻¹s⁻¹) | (10⁻⁴s⁻¹) | (10⁻⁹ M) |
| p1C11 scFv | 1.11 | 2.27 | 2.1 |
| c-p1C11 | 0.63 | 0.52 | 0.82 |
| VEGF | 1.87 | 1.81 | 0.93 |

*All rates are determined by surface plasmon resonance using BIAcore system, and are mean of at least three separate determinations.

### Example X

The present example demonstrates production of a VEGFR antagonist, namely, an anti-flt-1 (anti-VEGFR-1) monoclonal antibody, MF-1.

The rat anti-VEGFR-1 monoclonal antibody was developed through a standard hybridoma technique. Eight weeks old rats were primed intraperitoneally (i.p.) with 100 μg of VEGFR-1 Fc (constant region) recombinant protein (R&D Systems, Minneapolis, Minn.) mixed with complete Freunds adjuvant. Then, the rats were boosted three times prior to fusion with the same protein mixed with incomplete Freunds adjuvant.

Hybridoma cells were generated by fusing myeloma cells P3x63Ag8.653 with spleen cells and bone marrow cells from immunized rats. Anti-VEGFR-1 specific clones were selected using VEGFR-1 alkaline phosphatase (AP) recombinant protein in ELISA-based binding and blocking assays. Positive clones were subcloned by limiting dilution.

Anti-VEGFR-1 monoclonal antibodies (mAbs) from hybridomas were obtained via continuous feed fermentation in serum-free medium. The mAbs were purified from serum-free conditioned media by affinity chromatography using Gamma-bind protein G-Sepharose. The mAbs used in in vivo studies were tested for endotoxin using the Pyrogent Plus® Limulus Amebocyte Lysate kit (BioWhittaker, Walkersville, Md.). All antibody preparations used in animal studies contained ≦1.25 EU/ml of endotoxin. Anti-VEGFR-1 polyclonal antibodies were generated from recombinant VEGFR-1 AP protein immunized rabbit and purified by Gamma-bind protein G column (Amersham Pharmacia Biotech, Uppsala, Sweden).

The immunochemical properties of anti-VEGFR-1 mAbs were characterized in ELlSA-based binding and blocking assays as well as BIAcore analysis for affinity. Binding assays were performed by coating 96-well microliter plates (Falcon Flexible plate, Becton Dickinson, Bedford, Mass.) with 50 ng/well VEGFR-1 AP or VEGFR-2 AP protein overnight al 4° C. Wells were blocked by adding 200 μl of phosphate-buffered saline containing 5% bovine serum, 0.05% Tween 20 (blocking buffer) and incubating for 2 hrs at room temperature (RT). Wells were then washed (5×) and incubated for 1 hr at RT with various concentrations of mAbs at 50 μl diluted in blocking buffer. Wells were again washed (5×) and incubated with 50 μl of goat anti-rat IgG-HRP (BioSource International, Camarillo, Calif.) for 1 hr at RT. Wells were washed (5×) for a final time and then incubated with 50 μl of 3,3′, 5,5′-tetra-methylbenzidine (TMB) substrate (Kirkegaard and Perry Lab inc., Gaithersburg, Md.) for 15 mins at RT. The reaction was stopped by adding 50 μl of 1 M Phosphoric Acid (H₃PO₄) and wells read at 450 nm on a microtiter plate reader.

For VEGFR-1/VEGF or P1GF blocking assays, wells were coated with 100 ng of VEGF or P1GF (R&D Systems, Minneapolis, Minn.) overnight at 4° C. Wells are blocked as described above and then incubated for 1 hr at RT with 100 ng of VEGFR-1 AP that had been preincubated for 1 hr with various concentrations of mAb. Wells were washed and incubated with p-nitrophenyl phosphate (PNPP, Sigma, St. Louis, Mo.). Color was developed for 30 mins at RT and was then read at 405 nm on a microtiter plate reader.

The binding kinetics of anti-VEGFR-1 mAbs to VEGFR-1 was determined using BIAcore biosensor (Pharmacia Biosensor). VEGFR-1 Fc fusion protein was immobilized onto a sensor chip and the mAbs was injected at concentrations ranging from 3.125 nM to 50 nM. Sensorgrams were obtained at each concentration and were evaluated using the program, BIA Evaluation 2.0, to determine the ratio of rate constant kon/koff for Kd value.

### Example XI

The present example demonstrates the inhibition of tumor growth after administration of an epidermal growth factor receptor (EGFR) antagonist, the monoclonal antibody ERBITUX™ (also known as IMC-C225 or C225), and a therapeutically effective amount of a vascular endothelial growth factor receptor (VEGFR) antagonist, the anti-VEGFR-1 monoclonal antibody DC101.

Antibodies for immunohistochemical analysis and antiangiogenic therapy were obtained as follows: rat anti-mouse CD31/PECAM-1 antibody from Pharmingen (San Diego, Calif.); mouse anti-proliferating cell nuclear antigen (PCNA) clone PC10 DAKO A/S from DAKO Corp. (Carpinteria, Calif.); peroxidase-conjugated goat anti-rat immunoglobulin (IgG) (H+L) and Texas Red-conjugated goat anti-rat IgG from Jackson Research Laboratories (West Grove, Pa.); peroxidase-conjugated rat antimouse IgG2a from Serotec Harlan Bioproducts for Science, Inc. (Indianapolis, Ind.); and rat anti-mouse VEGF receptor-2 monoclonal antibody (Prewett et al., 1999; Witte et al., 1998) and chimeric anti-human EGF receptor monoclonal antibody (Goldstein et al., 1995) from ImClone Systems, Inc. (New York, N.Y.) (Prewett et al., 1999; Witte et al., 1998).

Eight-week-old male alhymic nude mice (National Cancer Institute, Animal Production Area, Frederick, Md.), were given an intraperitoneal injection of 1.0×10⁶ KM12L4 colon cancer cells in 500 μl of HBSS with a 30-gauge needle attached to a 1-ml syringe. Mice were then randomized into one of four treatment groups (10 mice per group): control, DC101, C225, or DC101 and C225. All animal studies were conducted under guidelines approved by the Animal Care and Use Committee of MD Anderson Cancer Center and UKCCCR, 1998).

Beginning 3 days after injection of tumor cells, mice were given intraperitoneal (i.p.) injections every third day of either control vehicle (phosphate buffered saline [PBS]), DC101 (0.8 mg), C225 (1.0 mg), or DC101 (0.8 mg) and C225 (1.0 mg) (each injection given in a 700-μl total volume) with a 27-gauge needle attached to a 1-ml syringe. Mice were weighed weekly. Mice were killed when the control group became moribund (about 30 days after therapy began), and complete necropsies were performed, tumor burden was quantified, and tumors were harvested and anlysed as described below.

For each mouse, necropsy was performed, the size of peritoneal tumors was measured with calipers, the mean tumor size was determined, and representative lesions were excised. The extent of ascites was assessed by an investigator who was unaware of the treatment-group assignment as follows: grade 0, no ascites; grade 1, small ascites; grade 2, moderate ascites; grade 3, large ascites; grade 4, massive ascites with tense abdomen (Aparicio et al., 1999). Tumor sections were either embedded in OCT compound and frozen at −70° C. or fixed in formalin and then embedded in paraffin.

Tissue sections were treated by standard deparaffinization (for formalin-fixed and paraffin-embedded tissues) or by fixation in acetone and chloroform (for tissues frozen in OCT), and immuno-histochemical analyses were performed as described previously (Shaheen et al., 1999). Briefly, endogenous peroxidases were blocked with 3% H₂O₂ in methanol, and the slides were washed with PBS, incubated for 20 min in protein-blocking solution (PBS supplemented with 1% normal goat serum and 5% normal horse serum), and incubated overnight at 4° C. with primary antibodies against CD31 or PCNA. Then, the slides were washed, incubated with protein-blocking solution, incubated for 1 h at room temperature with peroxidase-conjugated secondary antibodies, washed incubated with DAB, washed, counterstained with haematoxylin, washed, and mounted with Universal Mount and dried on a hot plate at 56° C. Frozen sections to be stained for CD31 were incubated with a secondary antibody conjugated to Texas Red (red fluorescence) instead of the peroxidase-conjugated antibody. Omission of the primary antibody served as negative control.

Terminal deoxynucletidyl transferase-(TdT)-mediated dUTP nick-end labelling (TUNEL) staining was performed according to the manufacturer's protocol. Briefly, the sections were fixed with 4% methanol-free paraformaldehyde, washed, permeabilized with 0.2% Triton X-100, washed, incubated with the kit's equilibration buffer, incubated with a reaction mix containing equilibration buffer, nucleotide mix, and the TdT enzyme at 37° C. for 1 h, incubated for 15 min at room temperature with 2× standard saline citrate to stop the TdT reaction, washed, stained with DAPI mount (to visualize the nuclei), and glass coverslips were applied.

Numbers of tumor vessels and PCNA-positive cells were evaluated by light microscopy (counted in five random 0.159-mm² fields at 100× magnification), imaged digitally, and processed with Optimas Image Analysis software (Biscan, Edmond, Wash.). Apoptotic cells were visualized with immunofluorescence as follows. Sections were digitally imaged and processed with Adobe Photoshop software (Adobe Systems, Mountain View, Calif.). CD311-positive endothelial cells (ECs) were detected by localized red fluorescence by using a rhodamine filter. Apoptosis was determined by localized green fluorescence for tumor cells (TCs) or green with red fluorescence for ECs by using a fluorescence filter. Nuclei were detected by the blue fluorescence of the DAPI with its filter. Cells were counted in five consecutive, non-overlapping fields 0.011-mm² fields per slide at 400× magnification with the first field selected at random in anon-necrotic portion of the tumor. The percentages of apoptotic ECs and TCs per field were then calculated as [% apoptotic cells=(number of apoptotic cells/total number of cells)×100].

Measuring the diameter for the peritoneal lesions 30 days after therapy began was used to assess gross tumor burden. The mean peritoneal tumor size was smaller in the DC101 group (50.3% smaller) and in the combination DC101+C225 group (66.7% smaller) than in the control group. Although 100% of the control and C225 mice had peritoneal disease at the end of the study, only 10% of the DC101 mice and 30% of the combination therapy mice showed no evidence of disease. Finally, the mean ascites grade was lower for both the DC101 (66.7% lower) and combination therapy groups (100% lower) than for the control mice. Moreover, the DC101+C225 group had significantly less ascites than did the DC101 group; virtually no ascites was found in the combination group.

Tumor cells were stained for PCNA by immunohistochemical analysis to assess tumor cell proliferation. Peritoneal tumors were smaller in the DC101 (50.3% smaller) and DC101+C225 groups (66.7% smaller) than in the control group.

Section of the peritoneal lesions for mice were also stained for CD31 immunofluorescence to detect the number of ECs as a measure of angiogenesis. Significantly fewer ECs was observed in the DC101 and DC101+C225 groups than in the control group.

Immunofluorescent TUNEL staining, with and without concurrent staining for CD31, was performed on sections of peritoneal metastases to quantify TC and EC apoptosis. More apoptotic TCs and ECs were observed in the DC101 group (14.3-fold more for TCs and 226-fold more for ECs) and DC101+C225 group (23.6-fold more for TCs and 331-fold more for ECs) than in the control group, and more apoptotic TCs and ECs were observed in the DC101+C225 group than in the DC101 group alone ((1.7-fold more for TCs and 1.46-fold more for ECs).

### Example XII

### Production of Human FAB

### Example XII(a)

### Proteins and Cell Lines

Primary-cultured HUVEC were obtained from Dr. S. Rafii at Cornell Medical Center, New York, and maintained in EBM-2 medium (Clonetics, Walkersville, Md.) at 37° C., 5% CO₂. The soluble fusion proteins, KDR (VEGFR-2)-AP, its immunoglobulin (Ig) domain-deletion variants, and Flk-1-AP, were expressed in stably transfected NIH 3T3 and purified from cell culture supernatants by affinity chromatography using immobilized monoclonal antibody to AP as described by Lu et al., J. Biol. Chem. 275: 14321-30 (2000). VEGF₁₆₅ protein was expressed in baculovirus and purified following the procedures described in Zhu et al., Cancer Res. 58: 3209-14 (1998). The leukemia cell lines, HL60 and HEL, were maintained in RPMI containing 10% fetal calf serum.

### Example XII(b)

### Phage ELISA

Individual TG1 clones were picked and grown at 37° C. in 96 well plates and rescued with M13K07 helper phage as described above. The amplified phage preparation was blocked with ⅙ volume of 18% milk/PBS at RT for 1 h and added to Maxi-sorp 96-well microtiter plates (Nunc) coated with KDR (VEGFR-2)-AP or AP (1 μg/ml×100 μl). After incubation at RT for 1 h the plates were washed 3 times with PBST and incubated with a rabbit anti-M13 phage-HRP conjugate (Amersham Pharmacia Biotech, Piscataway, N.J.). The plates were washed 5 times, TMB peroxidase substrate (KPL, Gaithersburg, Md.) added, and the absorbance at 450 nm read using a microplate reader (Molecular Devices, Sunnyvale, Calif.).

### Example XII(c)

### DNA BstN I Pattern Analysis and Nucleotide Sequencing

The diversity of the anti-KDR (VEGFR-2) Fab clones after each round of selection was analyzed by restriction enzyme digestion patterns (i.e., DNA fingerprints). The Fab gene insert of individual clones was PCR amplified using primers: PUC19 reverse, 5′ AGCGGATAACAATTTCACACAGG 3′; and fdtet seq, 5′ GTCGTCTTTCCAGACGTTAGT 3′. The amplified product was digested with a frequent-cutting enzyme, BstN I, and analyzed on a 3% agarose gel. DNA sequences of representative clones from each digestion pattern were determined by dideoxynucleotide sequencing.

### Example XII(d)

### Expression and Purification of Soluble Fab Fragments

Plasmids of individual clones were used to transform a nonsuppressor E. coli host HB2151. Expression of the Fab fragments in HB2151 was induced by culturing the cells in 2YTA medium containing 1 mM isopropyl-1-thio-β-D-galactopyranoside (IPTG, Sigma) at 30° C. A periplasmic extract of the cells was prepared by resuspending the cell pellet in 25 mM Tris (pH 7.5) containing 20% (w/v) sucrose, 200 mM NaCl, 1 mM EDTA and 0.1 mM PMSF, followed by incubation at 4° C. with gentle shaking for 1 h. After centrifugation at 15,000 rpm for 15 min, the soluble Fab protein was purified from the supernatant by affinity chromatography using a Protein G column followed the manufacturer's protocol (Amersham Pharmacia Biotech).

### Example XII(e)

### Selection of Human Anti-KDR (VEGFR-2) Fab from Phage Display Library

A large human Fab phage display library containing 3.7×10¹⁰ clones (DeHaard et al., J. Biol. Chem. 274: 18218-30 (1999)) was used for the selection. The library consists of PCR-amplified antibody variable light chain genes and variable heavy chain genes fused to human constant light chain genes (κ and λ) and DNA encoding the IgG1 heavy chain C_{H}1 domain, respectively. Both heavy and light chain constructs are preceded by a signal sequence—pelB for the light chain and gene III signal sequence for the heavy chain. Heavy chain constructs further encode a portion of the gene III protein for phage display, a hexahistidine tag, and an 11 amino-acid-long c-myc tag, followed by an amber codon (TAG). The hexahistidine and c-myc tags can be used for purification or detection. The amber codon allows for phage display using suppressor hosts (such as TG1 cells) or production of Fab fragments in soluble form when transformed into a nonsupressor host (such as HB2151 cells).

The library stock was grown to log phase, rescued with M13-KO7 helper phage and amplified overnight in 2YTAK medium (2YT containing 100 μg/ml of ampicillin and 50 μg/ml of kanamycin) at 30° C. The phage preparation was precipitated in 4% PEG/0.5M NaCl, resuspended in 3% fat-free milk/PBS containing 500 μg/ml of AP protein and incubated at 37° C. for 1 h to capture phage displaying anti-AP Fab fragments and to block other nonspecific binding.

KDR (VEGFR-2)-AP (10 μg/ml in PBS) coated Maxisorp Star tubes (Nunc, Rosklide, Denmark) were first blocked with 3% milk/PBS at 37° C. for 1 h, and then incubated with the phage preparation at RT for 1 h. The tubes were washed 10 times with PBST (PBS containing 0.1% Tween-20) followed by 10 times with PBS. Bound phage were eluted at RT for 10 min with 1 ml of a freshly prepared solution of 100 mM tri ethyl amine (Sigma, St. Louis, Mo.). The eluted phage were incubated with 10 ml of mid-log phase TG1 cells at 37° C. for 30 min stationary and 30 min shaking. The infected TG1 cells were pelleted and plated onto several large 2YTAG plates and incubated overnight at 30° C. All the colonies grown on the plates were scraped into 3 to 5 ml of 2YTAG medium, mixed with glycerol (10% final concentration), aliquoted and stored at −70° C. For the next round selection, 100 of the phage stock was added to 25 ml of 2YTAG medium and grown to mid-log phase. The culture was rescued with M13K07 helper phage, amplified, precipitated, and used for selection followed the procedure described above, with reduced concentrations of KDR (VEGFR-2)-AP immobilized on the immunotube and increased number of washes after the binding process.

A total of three rounds of selection were performed on immobilized KDR (VEGFR-2), with varying protein concentrations and number of washings after the initial binding process. After each round selection, 93 clones were randomly picked and tested by phage ELISA for binding to KDR (VEGFR-2). Seventy out of the 93 clones (75%) picked after the second selection, and greater than 90% of the recovered clones after the third selection were positive in KDR (VEGFR-2) binding, suggesting a high efficiency of the selection process. DNA segments encoding the Fab from all the 70 binders identified in the second selection were amplified, digested with BstN I, and compared for fingerprint patterns. A total of 42 different patterns were observed, indicating an excellent diversity of the isolated anti-KDR (VEGFR-2) Fab. Cross-reactivity examination demonstrated that 19 out of the 42 antibodies were specific FLT-1 (VEGFR-1)-binders, whereas the rest 23 antibodies bound to both KDR (VEGFR-2) and its murine homologue, Flk-1. Further selection was achieved with a competitive VEGF-binding assay in which the binding of soluble KDR (VEGFR-2) to immobilized VEGF in the presence or absence of the anti-KDR (VEGFR-2) Fab fragments was determined. The assay identified four Fab clones that were capable of blocking the binding between VEGF and KDR (VEGFR-2). Three were KDR (VEGFR-2)-specific binders and one cross-reacted with Flk-1. DNA fingerprinting and sequencing analysis confirmed that all four KDR (VEGFR-2/VEGF blocking antibodies were different with unique DNA and amino acid sequences.

The amino acid sequences for CDR1, CDR2 and CDR3 of V_{H} and V_{L} for the four clones are given in Table 3.

TABLE 3

CDR SEQUENCES OF SELECTED KDR (VEGFR-2)-
BINDING HUMAN FABS

| | | | |
|---|---|---|---|
| Clone | CDR1 | CDR2 | CDR3 |

Light Chain

| | | | |
|---|---|---|---|
| D2C6 | RASQSVSSYLA | DSSNRAT | LQHNTFPPT |
| | (SEQ ID NO:1) | (SEQ ID NO:2) | (SEQ ID NO:3) |
| D2H2 | RASQGISSRLA | AASSLQT | QQANRFPPT |
| | (SEQ ID NO:4) | (SEQ ID NO:5) | (SEQ ID NO:6) |
| D1H4 | AGTTTDLTYYDLVS | DGNKRPS | NSYVSSRFYV |
| | (SEQ ID NO:7) | (SEQ ID NO:8) | (SEQ ID NO:9) |
| D1F7 | SGSTSNIGTNTAN | NNNQRPS | AAWDDSLNGHWV |
| | (SEQ ID NO:10) | (SEQ ID NO:11) | (SEQ ID NO:12) |

Heavy Chain

| | | | |
|---|---|---|---|
| D2C6 | GFTFSSYSMN | SISSSSSYIYYADS | VTDAFDI |
| | (SEQ ID NO:13) | VKG | (SEQ ID NO:15) |
| | | (SEQ ID NO:14) | |
| D2H2 | GFTFSSYSMN | SISSSSSYIYYADS | VTDAFDI |
| | | VKG | |
| D1H4 | GFTFSSYSMN | SISSSSSYIYYADS | VTDAFDI |
| | | VKG | |
| D1F7 | GGTFSSYAIS | GGIIPIFGTANYAQ | GYDYYDSSGVASP |
| | (SEQ ID NO:16) | KFQG | FDY |
| | | (SEQ ID NO:17) | (SEQ ID NO:18) |

Complete sequences for the VH and VL chains are presented in the Sequence Listing. For D1F7, the nucleotide and amino acid sequences for V_{H} are represented by SEQ ID NOS: 19 and 20 respectively, and the nucleotide and amino acid sequences for V_{L} are represented by SEQ ID NOS: 21 and 22.

For D2C6, the nucleotide and amino acid sequences for V_{H} are represented by SEQ ID NOS: 23 and 24 respectively, and the nucleotide and amino acid sequences for V_{L} are represented by SEQ ID NOS: 25 and 26.

For D2H2, the nucleotide and amino acid sequences for V_{H} are represented by SEQ ID NOS: 30 and 31 respectively, and the nucleotide and amino acid sequences for V_{L} are represented by SEQ ID NOS: 32 and 33.

For D1H4, the nucleotide and amino acid sequences for V_{H} are represented by SEQ ID NOS: 27 and 24 respectively, and the nucleotide and amino acid sequences for V_{L} are represented by SEQ ID NOS: 28 and 29.

A second library was created combining the single heavy chain of D2C6 with a diverse population of light chains derived from the original library. Ten additional Fabs were identified, designated SA1, SA3, SB10, SB5, SC7, SD2, SD5, SF2, SF7, and 1121. The nucleotide and amino acid sequences for V_{L} of the ten Fabs are represented as follows. For SA1, the nucleotide and amino acid sequences for V_{L} are represented by SEQ ID NOS: 34 and 35. For SA3, the nucleotide and amino acid sequences for V_{L} are represented by SEQ ID NOS: 36 and 37. For SB10, the nucleotide and amino acid sequences for V_{L} are represented by SEQ ID NOS: 38 and 39. For SB5, the nucleotide and amino acid sequences for V_{L} are represented by SEQ ID NOS: 40 and 41. For SC7, the nucleotide and amino acid sequences for V_{L} are represented by SEQ ID NOS: 42 and 43. For SD2, the nucleotide and amino acid sequences for V_{L} are represented by SEQ ID NOS: 44 and 45. For SDS, the nucleotide and amino acid sequences for V_{L} are represented by SEQ ID NOS: 46 and 47. For SF2, the nucleotide and amino acid sequences for V_{L} are represented by SEQ ID NOS: 48 and 49. For SF7, the nucleotide and amino acid sequences for V_{L} are represented by SEQ ID NOS: 50 and 51. For 1121, the nucleotide and amino acid sequences for V_{L} are represented by SEQ ID NOS: 52 and 53.

The V_{L} CDR sequences are presented in Table 4.

TABLE 4

LIGHT CHAIN CDR SEQUENCES OF KDR (VEGFR-
2)-BINDING HUMAN FABS

| | | | |
|---|---|---|---|
| Clone | CDR1 | CDR2 | CDR3 |

| | | | |
|---|---|---|---|
| SA1 | TGSHSNFGAGTDV | GDSNRPS | QSYDYGLRGWV |
| | (SEQ ID NO:54) | (SEQ ID NO:55) | (SEQ ID NO:56) |
| SA3 | RASQNINNYLN | AASTLQS | QQYSRYPPT |
| | (SEQ ID NO:57) | (SEQ ID NO:58) | (SEQ ID NO:59) |
| SB10 | TGSSTDVGNYNYIS | DVTSRPS | NSYSATDTLV |
| | (SEQ ID NO:60) | (SEQ ID NO:61) | (SEQ ID NO:62) |
| SB5 | TGQSSNIGADYDVH | GHNNRPS | QSYDSSLSGLV |
| | (SEQ ID NO:63) | (SEQ ID NO:64) | (SEQ ID NO:65) |
| SC7 | RASQDISWLA | AASLLQS | QQADSFPPT |
| | (SEQ ID NO:66) | (SEQ ID NO:67) | (SEQ ID NO:68) |
| SD2 | RASQSIKRWLA | AASTLQS | QQANSFPPT |
| | (SEQ ID NO:69) | (SEQ ID NO:70) | (SEQ ID NO:71) |
| SD5 | SGSRSNIGAHYEVQ | GDTNRPS | QSYDTSLRGPV |
| | (SEQ ID NO:72) | (SEQ ID NO:73) | (SEQ ID NO:74) |
| SF2 | TGSSSNIGTGYDVH | AYTNRPS | QSFDDSLNGLV |
| | (SEQ ID NO:75) | (SEQ ID NO:76) | (SEQ ID NO:77) |
| SF7 | TGSHSNFGAGTDVH | GDTHRPS | QSYDYGLRGWV |
| | (SEQ ID NO:78) | (SEQ ID NO:79) | (SEQ ID NO:80) |
| 1121 | RASQGIDNWLG | DASNLDT | QQAKAFPPT |
| | (SEQ ID NO:81) | (SEQ ID NO:82) | (SEQ ID NO:83) |

### Example XIII

### Assays

### Example XIII(a)

### Quantitative KDR (VEGFR-2) Binding and Blocking of KDR (VEGFR-2)/VEGF Interaction

In a direct binding assay, various amounts of soluble Fab proteins were added to KDR (VEGFR-2)-coated 96-well Maxi-sorp microtiter plates and incubated at RT for 1 h, after which the plates were washed 3 times with PBST. The plates were then incubated at RT for 1 h with 100 μl of a rabbit anti-human Fab antibody-HRP conjugate (Jackson ImmunoResearch Laboratory Inc., West Grove, Pa.). The plates were washed and developed following the procedure described above for the phage ELISA. In a competitive KDR (VEGFR-2)/VEGF blocking assay, various amounts of Fab proteins were mixed with a fixed amount of KDR (VEGFR-2)-AP (100 ng) and incubated at RT for 1 h. The mixtures were then transferred to 96-well microtiter plates precoated with VEGF₁₆₅ (200 ng/well) and incubated at RT for an additional 2 h, after which the plates were washed 5 times and the substrate for AP (p-nitrophenyl phosphate, Sigma) was added. Absorbance at 405 nm was measured to quantify the bound KDR (VEGFR-2)-AP molecules (8). IC₅₀, i.e., the Fab protein concentration required for 50% inhibition of KDR (VEGFR-2) binding to VEGF, was then calculated.

The four VEGF-blocking clones (D2C6, D2H2, D1H4, D1F7) were expressed as soluble Fab and purified from periplasmic extracts of E. coli by Protein G affinity chromatography. The yield of purified Fab proteins of these clones ranged from 60 to 400 μg/liter culture. SDS-PAGE analysis of each purified Fab preparation yielded a single protein band with expected molecular size.

Clone D2C6 and D2H2 are more efficient binders, followed by clone D1H4 and D1F7. All four Fabs also block KDR (VEGFR-2) binding to immobilized VEGF. The antibody concentrations required for 50% of inhibition of KDR (VEGFR-2) binding to VEGF are approximately 2 nM for clones D2C6, D2H2, and D1H4 and 20 nM for clone D1F7. Only clone D1F7 blocks VEGF from binding to Flk-1, with an IC₅₀ of approximately 15 nM.

### Example XIII (b)

### BIAcore Analysis of the Soluble scFv

The binding kinetics of soluble Fab proteins to KDR (VEGFR-2) were measured by surface plasmon resonance using a BIAcore biosensor (Pharmacia Biosensor). KDR (VEGFR-2)-AP fusion protein was immobilized onto a sensor chip and soluble Fab proteins were injected at concentrations ranging from 1.5 nM to 100 nM. Sensorgrams were obtained at each concentration and were evaluated using a program, BIA Evaluation 2.0, to determine the rate constants kon and koff. Kd was calculated from the ratio of rate constants koff/kon.

All three KDR (VEGFR-2)-specific Fab fragments bind to immobilized receptor with Kd of 2 to 4 nM (Table 5). The cross-reactive clone, D1F7, has a Kd of 45 nM, which is about 10- to 15-fold weaker than those of the KDR (VEGFR-2)-specific clones. It is noteworthy that, although the overall Kd for the three KDR (VEGFR-2)-specific Fab fragments are similar, the individual binding kinetics, i.e., the kon and koff for these antibodies are quite different, e.g., D2C6 possesses the fastest on-rate, while D1H4 has the slowest off-rate (Table 5).

TABLE 5
BINDING KINETICS OF THE FOUR
NEUTRALIZING HUMAN ANTI-KDR (VEGFR-2) FAB
FRAGMENTS

| | kon | koff | Kd |
|---|---|---|---|
| Clone | (10⁴ M⁻¹S⁻¹) | (10⁻⁴S⁻¹) | (nM) |
| Hu-2C6 Fab | 27.3 ± 8.6* | 5.38 ± 0.54 | 1.97 |
| Hu-2H2 Fab | 12.4 ± 2.9 | 4.87 ± 0.18 | 3.93 |
| Hu-1H4 Fab | 5.55 ± 0.59 | 1.53 ± 0.22 | 2.76 |
| Hu-1F7 Fab | 4.14 ± 1.21 | 18.7 ± 2.12 | 45.2 |

All numbers are determined by BIAcore analysis and represent the mean±SE from at least three separate determinations.

### Example XIII(c)

### Binding Epitope Mapping

The production of KDR (VEGFR-2) extracellular Ig-like domain deletion variants has been previously described (Lu et al. (2000)). In an epitope-mapping assay, full length KDR (VEGFR-2)-AP, Ap fusions of two KDR (VEGFR-2) Ig-domain deletion variants, and Flk-1-AP were first immobilized onto a 96-well plate (Nunc) using a rabbit anti-AP antibody (DAKO-immunoglobulins, Glostrup, Denmark) as the capture reagent. The plate was then incubated with various anti-KDR (VEGFR-2) Fab proteins at RT for 1 h, followed by incubation with a rabbit anti-human Fab antibody-HRP conjugate. The plate was washed and developed as described above.

The binding epitopes of the anti-KDR (VEGFR-2) Fab fragments were mapped using the full-length KDR (VEGFR-2) and two KDR (VEGFR-2) Ig domain-deletion variants. KDR (VEGFR-2)(1-3) is a KDR (VEGFR-2) variant containing the first three N-terminal Ig domains. KDR (VEGFR-2)(3) is a variant containing only the third Ig domain. Clones D2C6 and D1H4 bind equally well to KDR (VEGFR-2), KDR (VEGFR-2)(1-3) and KDR (VEGFR-2)(3), thus locating their binding epitope(s) within Ig domain 3. Clones D2H2 and D1F7 bind much more efficiently to full-length KDR (VEGFR-2) and KDR (VEGFR-2)(1-3), indicating a broader binding epitope(s) within KDR (VEGFR-2) Ig domains 1 to 3. Only clone DIF7 cross-reacts with Flk-1.

### Example XIII(d)

### Anti-Mitogenic Assay

HUVEC (5×10³ cells/well) were plated onto 96-well tissue culture plates (Wallach, Inc., Gaithersburg, Md.) in 200 μl of EBM-2 medium without VEGF, basic fibroblast growth factor (bFGF) or epidermal growth factor (EGF) and incubated at 37° C. for 72 h. Various amounts of Fab proteins were added to duplicate wells and pre-incubated at 37° C. for 1 h, after which VEGF₁₆₅ was added to a final concentration of 16 ng/ml. After 18 h of incubation, 0.25 μCi of [³H]TdR (Amersham) was added to each well and incubated for an additional 4 h. The cells were washed once with PBS, trypsinized and harvested onto a glass filter (Printed Filtermat A, Walach) with a cell harvester (Harvester 96, MACH III, TOMTEC, Orange, Conn.). The membrane was washed three times with H₂O and air-dried. Scintillation fluid was added and DNA incorporated radioactivity was determined on a scintillation counter (Wallach, Model 1450 Microbeta Scintillation Counter).

The ability of human anti-KDR (VEGFR-2) Fab to block VEGF-stimulated mitogenic activity on HUVEC. All four human Fab fragments inhibited VEGF induced DNA synthesis in HUVEC in a dose-dependent manner. The Fab concentration that inhibited 50% (EC₅₀) of VEGF-stimulated [³H]-TdR incorporation in HUVEC, is approximately 0.5 nM for clones D2C6 and D1H4, 0.8 nM for clone D2H2, and 15 nM for clone D1F7. Controls included VEGF only (1500 cpm) and plain medium (60 cpm). Duplicate wells were assayed. The data shown are representative of at least three separate experiments.

### Example XIII(e)

### Leukemia Migration Assay

HL60 and HEL cells were washed three times with serum-free plain RPMI 1640 medium and suspended in the medium at 1×10⁶/ml. Aliquots of 100 μl cell suspension were added to either 3-μm-pore transwell inserts for HL60 cells, or 8-μm-pore transwell inserts for HEL cells (Costar®, Corning Incorporated, Corning, N.Y.) and incubated with the anti-KDR (VEGFR-2) Fab proteins (5 μg/ml) for 30 min at 37° C. The inserts were then placed into the wells of 24-well plates containing 0.5 ml of serum-free RPMI 1640 with or without VEGF₁₆₅. The migration was carried out at 37° C., 5% CO₂ for 16-18 h for HL60 cells, or for 4 h for HEL cells. Migrated cells were collected from the lower compartments and counted with a Coulter counter (Model Z1, Coulter Electronics Ltd., Luton, England).

VEGF induced migration of HL60 and HEL cells in a dose-dependent manner with maximum stimulation achieved at 200 ng/. All the anti-KDR (VEGFR-2) Fab fragments significantly inhibited VEGF-stimulated migration of HL60 and HEL cells. As a control, a Fab fragment of C225, an antibody directed against EGF receptor, did not show significant inhibitory effect in this assay.

### Example XIV

### Production of IgG

### Example XIV(a)

### Construction of Vectors for Expression of IgG

Separate vectors for expression of IgG light chain and heavy chains were constructed. Cloned V_{L} genes were digested and ligated into the vector pKN100. Cloned V_{H} genes were digested and ligated into the vector pGID105 containing the human IgG1 (γ) heavy chain constant domain. Constructs were examined by restriction enzyme digestion and verified by dideoxynucleotide sequencing. In both cases, expression is under control of the HCMV promoter and terminated by an artificial termination sequence.

The assembled heavy and light chain genes were then cloned into Lonza GS expression vectors pEE6.1 and pEE12.1. Heavy and light chain vectors were recombined into a single vector for stable transfection of CHO cells and NS0 cells. Transfected cells are cultured in glutamine minus medium and express antibodies at levels as high as 1 g/L.

What is claimed is:

1. A method of inhibiting tumor growth comprising administering to a human a therapeutically effective amount of a vascular endothelial growth factor receptor (VEGFR) antibody or functional equivalent thereof that specifically binds to VEGFR and inhibits binding of a ligand thereto and a therapeutically effective amount of a separate epidermal growth factor receptor (EGFR) antibody or functional equivalent thereof that specifically binds to EGFR and inhibits binding of ligand thereto.

2. The method of claim 1, wherein the tumor overexpresses VEGFR.

3. The method of claim 1, wherein the tumor is a tumor of the colon.

4. The method of claim 1, wherein the tumor is a non-small cell lung carcinoma (NSCLC).

5. The method of claim 1, wherein the VEGFR antibody is administered intravenously.

6. The method of claim 1, wherein the VEGFR antibody is administered orally.

7. The method of claim 1, wherein the VEGFR antibody is an antibody that specifically binds to fins-like tyrosine kinase receptor (flt-1) VEGFR-1 and inhibits binding of a ligand thereto.

8. The method of claim 1, wherein the VEGFR antibody, the EGFR antibody or both comprises a constant region of a human antibody.

9. The method of claim 8, wherein the VEGFR antibody, the EGFR antibody or both is a chimeric antibody comprising a variable region of a mouse antibody.

10. The method of claim 8, wherein the VEGFR antibody, the EGFR antibody or both is a humanized antibody comprising a variable region having complementarity-determining regions (CDRs) of a mouse antibody and framework regions of a human antibody.

11. The method of claim 8, wherein the VEGFR antibody, the EGFR antibody or both is a human antibody comprising a variable region of a human antibody.

12. The method of claim 1, wherein the tumor overexpresses EGFR.

13. The method of claim 1, wherein the EGFR antibody is administered intravenously.

14. The method of claim 1, wherein the EGFR antibody is administered orally.

15. The method of claim 1, wherein the method further comprises administering a chemotherapeutic agent or radiation.

16. A kit for inhibiting tumor growth comprising a therapeutically effective amount of an epidermal growth factor receptor (EGFR) antibody or functional equivalent thereof that specifically binds to EGFR and inhibits binding of a ligand thereto and a therapeutically effective amount of a separate vascular endothelial growth factor receptor (VEGFR) antibody or functional equivalent thereof that specifically binds to VEGFR and inhibits binding of a ligand thereto.

17. The kit of claim 16, wherein the kit further comprises a chemotherapeutic agent.

18. The method of claim 1, where in the VEGFR antibody is an antibody that specifically binds to kinase insert domain-containing receptor (KDR) and inhibits binding of a ligand thereto.

19. The method of claim 1, where in the VEGFR antibody is an antibody that specifically binds to fetal liver kinase receptor (flk-1) VEGFR-1 and inhibits binding of a ligand thereto.
